SEQUENCE LISTING (274 sequences)

SEQ ID NO: 1 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 2 - protein, 165 aa, Homo sapiens
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 3 - protein, 166 aa, Homo sapiens [sequence omitted]

SEQ ID NO: 4 - protein, 189 aa, Homo sapiens
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 5 - protein, 189 aa, Homo sapiens
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 6 - protein, 189 aa, Homo sapiens
MALPFALLMALVVLSCKSSCSLDCDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 7 - protein, 189 aa, Homo sapiens
MARSFSLLMAVLVLSYKSICSLGCDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLTEKKYSPCAWEVVRAEIMRSFSFSTNLKKGLRRKD

SEQ ID NO: 8 - protein, 189 aa, Homo sapiens
MALTFYLMVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLEVLCDQEVGVIESPLMYEDSILAVRKYFQRITLYLTEKKYSSCAWEVVRAEIMRSFSLSINLQKRLKSKE

SEQ ID NO: 9 - protein, 189 aa, Homo sapiens
MALSFSLLMAVLVLSYKSICSLGCDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 10 - protein, 189 aa, Homo sapiens
MASPFALLMALVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 11 - protein, 189 aa, Homo sapiens
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSFSFSTNLQKRLRRKD

SEQ ID NO: 12 - protein, 189 aa, Homo sapiens
MALSFSLLMAVLVLSYKSICSLGCDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 13 - protein, 189 aa, Homo sapiens
MALSFSLLMAVLVLSYKSICSLGCDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 14 - protein, 189 aa, Homo sapiens
MALSFSLLMAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDMEACVIQEVGVEETPLMNVDSILAVKKYFQRITLYLTEKKYSPCAWEVVRAEIMRSFSLSKIFQERLRRKE

SEQ ID NO: 15 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 16 - protein, 207 aa, Homo sapiens
MSTKPDMIQKCLWLEILMGIFIAGTLSLDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 17 - protein, 208 aa, Homo sapiens
MIIKHFFGTVLVLLASTTIFSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 18 - protein, 195 aa, Homo sapiens
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 19 - protein, 166 aa, Artificial
  synthetic
MSYNQLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 20 - protein, 166 aa, Artificial
  synthetic
MSYNQLGELQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 21 - protein, 166 aa, Artificial
  synthetic
MSYNQLGELQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSLHLKRYYGRILHYLKAKEYSHCAWTIVRVEILRNFYFINRLTGYLRN

SEQ ID NO: 22 - protein, 166 aa, Artificial
  synthetic
MSYNQLGELQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKEMSSLHLKRYYGRILHYLKAKEYSHCAWTIVRVEILRNFYFINRLTGYLRN

SEQ ID NO: 23 - protein, 166 aa, Artificial
  synthetic
MSYNLLGELQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 24 - protein, 166 aa, Artificial
  synthetic
MSYNQLGELQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 25 - protein, 166 aa, Artificial
  synthetic
MSYNQLGELQRSSNFQSQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKEMSSRHLKRYYGRILHYLKAKEYSHCAWTIVRVEILRNFYFINRLTGYLRN

SEQ ID NO: 26 - protein, 180 aa, Artificial
  synthetic
LDCNLLNNHLRRVTRQNLRHLSSMSNSFPVECLRENIAFELPQEFLQQTQPNKRDIKKAFYEMSLQAFNIFSQHTSKAWKERHLKQIQTGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 27 - protein, 180 aa, Artificial
  synthetic
LDCNLLNVHLRRVTRQNLRHLSSMSNSFPVECLRENNAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRALSYFTKFTALFRRK

SEQ ID NO: 28 - protein, 180 aa, Artificial
  synthetic
LDCNLLNVHLRRVTRQNLRHLSSMSNSFPVECLRENIAFELPQEFLQYTQPMKRDIKKAFYEMSLQAFNIFSQHTSKAWKERHLKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 29 - protein, 180 aa, Artificial
  synthetic
LDCNLLNVHLRRVTWQNLRHLSSMSNSFPVECLRENNAFELPQEFLQQTQPNKRDIKKAFYEMSLQAFNIFSQHTSKAWKERHLKQIQIGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 30 - protein, 180 aa, Artificial
  synthetic
LDCNLLNVHLRRVTRQNLRHLSSMSNSFPVECLRENNAFELPQEFLQYTQPMKRDIKKAFYEMSLQAFNIFSQHTSKAWKERHLKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 31 - protein, 166 aa, Homo sapiens
CDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 32 - protein, 165 aa, Homo sapiens; identical to SEQ ID NO: 2

SEQ ID NO: 33 - protein, 165 aa, Homo sapiens [sequence omitted]

SEQ ID NO: 34 - protein, 166 aa, Homo sapiens
CDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 35 - protein, 166 aa, Homo sapiens
CDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 36 - protein, 166 aa, Homo sapiens
CDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 37 - protein, 166 aa, Homo sapiens
CDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLTEKKYSPCAWEVVRAEIMRSFSFSTNLKKGLRRKD

SEQ ID NO: 38 - protein, 166 aa, Homo sapiens
CDLPQTHSLGNRRALILLAQMRRISPFSCLKDRHDFEFPQEEFDDKQFQKAQAISVLHEMIQQTFNLFSTKDSSAALDETLLDEFYIELDQQLNDLEVLCDQEVGVIESPLMYEDSILAVRKYFQRITLYLTEKKYSSCAWEVVRAEIMRSFSLSINLQKRLKSKE

SEQ ID NO: 39 - protein, 166 aa, Homo sapiens
CDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 40 - protein, 166 aa, Homo sapiens; identical to SEQ ID NO: 31

SEQ ID NO: 41 - protein, 166 aa, Homo sapiens
CNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSFSFSTNLQKRLRRKD

SEQ ID NO: 42 - protein, 166 aa, Homo sapiens
CDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 43 - protein, 166 aa, Homo sapiens
CDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 44 - protein, 166 aa, Homo sapiens
CDLPQTHSLGNRRALILLAQMGRISPFSCLKDRHDFGFPQEEFDGNQFQKAQAISVLHEMIQQTFNLFSTKDSSATWEQSLLEKFSTELNQQLNDMEACVIQEVGVEETPLMNVDSILAVKKYFQRITLYLTEKKYSPCAWEVVRAEIMRSFSLSKIFQERLRRKE

SEQ ID NO: 45 - protein, 179 aa, Homo sapiens
LDCNLLNVHLRRVTWQNLRHLSSMSNSFPVECLRENIAFELPQEFLQYTQPMKRDIKKAFYEMSLQAFNIFSQHTFKYWKERHKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 46 - protein, 166 aa, Homo sapiens; identical to SEQ ID NO: 15

SEQ ID NO: 47 - protein, 152 aa, Homo sapiens
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGMDPIVTVKKYFQGIYDYLQEKGYSDCAWEIVRVEMMRALTVSTTLQKRLTK

SEQ ID NO: 48 - protein, 165 aa, Homo sapiens; identical to SEQ ID NO: 2

SEQ ID NO: 49 - protein, 166 aa, Artificial
  synthetic
MSYNQLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 50 - protein, 166 aa, Artificial
  synthetic
MSYNQLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 51 - protein, 166 aa, Artificial
  synthetic
MSYNLLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 52 - protein, 166 aa, Artificial
  synthetic
MSYNLLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 53 - protein, 166 aa, Artificial
  synthetic
MSYNLLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 54 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 55 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 56 - protein, 166 aa, Artificial
  synthetic
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSLHLKRYYGRILHYLKAKEYSHCAWTIVRVEILRNFYFINRLTGYLRN

SEQ ID NO: 57 - protein, 166 aa, Artificial
  synthetic
MSYNQLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSLHLKRYYGRILHYLKAKEYSHCAWTIVRVEILRNFYFINRLTGYLRN

SEQ ID NO: 58 - protein, 166 aa, Artificial
  synthetic
MSYNQLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKLMSSLHLKRYYGRILHYLKAKEYSHCAWTIVRVEILRNFYFINRLTGYLRN

SEQ ID NO: 59 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSLHLKRYYGRILHYLKAKEYSHCAWTIVRVEILRNFYFINRLTGYLRN

SEQ ID NO: 60 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKLMSSLHLKRYYGRILHYLKAKEYSHCAWTIVRVEILRNFYFINRLTGYLRN

SEQ ID NO: 61 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKEMSSLHLKRYYGRILHYLKAKEYSHCAWTIVRVEILRNFYFINRLTGYLRN

SEQ ID NO: 62 - protein, 166 aa, Artificial
  synthetic
MSYNQLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 63 - protein, 166 aa, Artificial
  synthetic
MSYNQLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 64 - protein, 166 aa, Artificial
  synthetic
MSYNLLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 65 - protein, 166 aa, Artificial
  synthetic
MSYNLLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 66 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 67 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKLMSSRHLKRYYGRILHYLKAKEYSHCAWTIVRVEILRNFYFINRLTGYLRN

SEQ ID NO: 68 - protein, 166 aa, Artificial
  synthetic
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 69 - protein, 166 aa, Artificial
  synthetic
MSYNQLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 70 - protein, 166 aa, Artificial
  synthetic
MSYNQLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 71 - protein, 166 aa, Artificial
  synthetic
MSYNLLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 72 - protein, 166 aa, Artificial
  synthetic
MSYNLLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 73 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 74 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKEMSSLHLKRYYGRILHYLKAKEYSHCAWTIVRVEILRNFYFINRLTGYLRN

SEQ ID NO: 75 - protein, 166 aa, Artificial
  synthetic
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSRHLKRYYGRILHYLKAKEYSHCAWTIVRVEILRNFYFINRLTGYLRN

SEQ ID NO: 76 - protein, 166 aa, Artificial
  synthetic
MSYNQLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSRHLKRYYGRILHYLKAKEYSHCAWTIVRVEILRNFYFINRLTGYLRN

SEQ ID NO: 77 - protein, 166 aa, Artificial
  synthetic
MSYNLLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSRHLKRYYGRILHYLKAKEYSHCAWTIVRVEILRNFYFINRLTGYLRN

SEQ ID NO: 78 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKLMSSRHLKRYYGRILHYLKAKEYSHCAWTIVRVEILRNFYFINRLTGYLRN

SEQ ID NO: 79 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKLMSSRHLKRYYGRILHYLKAKEYSHCAWTIVRVEILRNFYFINRLTGYLRN

SEQ ID NO: 80 - protein, 166 aa, Artificial
  synthetic
MSYNQLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKEMSSRHLKRYYGRILHYLKAKEYSHCAWTIVRVEILRNFYFINRLTGYLRN

SEQ ID NO: 81 - protein, 166 aa, Artificial
  synthetic
MSYNQLGFLQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKEMSSRHLKRYYGRILHYLKAKEYSHCAWTIVRVEILRNFYFINRLTGYLRN

SEQ ID NO: 82 - protein, 166 aa, Artificial
  synthetic
MSYNLLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKEMSSRHLKRYYGRILHYLKAKEYSHCAWTIVRVEILRNFYFINRLTGYLRN

SEQ ID NO: 83 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKEMSSRHLKRYYGRILHYLKAKEYSHCAWTIVRVEILRNFYFINRLTGYLRN

SEQ ID NO: 84 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDFTRGKEMSSRHLKRYYGRILHYLKAKEYSHCAWTIVRVEILRNFYFINRLTGYLRN

SEQ ID NO: 85 - protein, 166 aa, Artificial
  synthetic
MSYNQLGELQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDNTRGKEMSSRHLKRYYGRILHYLKAKEYSHCAWTIVRVEILRNFYFINRLTGYLRN

SEQ ID NO: 86 - protein, 166 aa, Artificial
  synthetic
MSYNQLGELQRSSNFQCQKLLWQLNGRLEYCLKDRMNFDIPEEIKQKQQFQKEDAALTIYEMLQNIFAIFRQDSSSTGWNETIVENLLANVYHQINHLKTVLEEKLEKEDNTRGKEMSSRHLKRYYGRILHYLKAKEYSHCAWTIVRVEILRNFYFINRLTGYLRN

SEQ ID NO: 87 - protein, 166 aa, Artificial
  synthetic
MSYNLLGFLQRSSNFQCQKLLWQLNGRLEYCLKDRMNFDIPEEIKQSQQSQKEDAALTIYEMLQNIFAIFRQDSSSTGWNETIVENLLANVYHQINHLKTVLEEKSEKEDSTRGKSMSSSHLKRYYGRILHYLKAKEYSHCAWTIVRVEILRNFYFINRLTGYLRN

SEQ ID NO: 88 - protein, 180 aa, Homo sapiens
LDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 89 - protein, 180 aa, Artificial; identical to SEQ ID NO: 26
  synthetic

SEQ ID NO: 90 - protein, 180 aa, Artificial; identical to SEQ ID NO: 26
  synthetic

SEQ ID NO: 91 - protein, 180 aa, Artificial
  synthetic
LDCNLLNNHLRRVTRQNLRHLSSMSNSQPRECLRENNAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRALSYFTKFTALFRRK

SEQ ID NO: 92 - protein, 180 aa, Artificial
  synthetic
LDCNLLNNHLRRVTWQNLRHLSSMSNSFPVECLRENIAFELPQEFLQQTQPNKRDIKKAFYEMSLQAFNIFSQHTFKYWKERHLKQIQTGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 93 - protein, 180 aa, Artificial
  synthetic
LDCNLLNNHLRRVTWQNLRHLSSMSNSFPVECLRENIAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRALSYFTKFTALFRRK

SEQ ID NO: 94 - protein, 180 aa, Artificial
  synthetic
LDCNLLNNHLRRVTWQNLRHLSSMSNSQPRECLRENIAFELPQEFLQQTQPNKRDIKKAFYEMSLQAFNIFSQHTSKAWKERHLKQIQTGLDQQAEYLNQCLEEDENENEDMKEMKENEMKPSEARVPQLSSLELRRYFHRIDNFLKEKKYSDCAWEIVRVEIRRALSYFTKFTALFRRK

SEQ ID NO: 95 - protein, 180 aa, Artificial
  synthetic
LDCNLLNNHLRRVTWQNLRHLSSMSNSQPRECLRENIAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRALSYFTKFTALFRRK

SEQ ID NO: 96 - protein, 180 aa, Artificial; identical to SEQ ID NO: 95
  synthetic

SEQ ID NO: 97 - protein, 180 aa, Artificial
  synthetic
LDCNLLNNHLRRVTWQNLRHLSSMSNSQPRECLRENNAFELPQEFLQQTQPNKRDIKKAFYEMSLQAFNIFSQHTSKAWKERHLKQIQTGLDQQAEYLNQCLEEDENENEDMKEMKENEMKPSEARVPQLSSLELRRYFHRIDNFLKEKKYSDCAWEIVRVEIRRALSYFTKFTALFRRK

SEQ ID NO: 98 - protein, 180 aa, Artificial
  synthetic
LDCNLLNVHLRRVTRQNLRHLSSMSNSFPVECLRENIAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 99 - protein, 180 aa, Artificial
  synthetic
LDCNLLNVHLRRVTRQNLRHLSSMSNSFPVECLRENIAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 100 - protein, 180 aa, Artificial; identical to SEQ ID NO: 99
  synthetic

SEQ ID NO: 101 - protein, 180 aa, Artificial
  synthetic
LDCNLLNVHLRRVTRQNLRHLSSMSNSFPVECLRENIAFELPQEFLQYTQPMKRDIKKAFYEMSLQAFNIFSQHTSKAWKERHLKQIQIGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 102 - protein, 180 aa, Artificial
  synthetic
LDCNLLNVHLRRVTRQNLRHLSSMSNSFPVECLRENIAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRALSYFTKFTALFRRK

SEQ ID NO: 103 - protein, 180 aa, Artificial
  synthetic
LDCNLLNVHLRRVTRQNLRHLSSMSNSFPVECLRENIAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 104 - protein, 180 aa, Artificial
  synthetic
LDCNLLNVHLRRVTRQNLRHLSSMSNSFPVECLRENNAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 105 - protein, 180 aa, Artificial
  synthetic
LDCNLLNVHLRRVTWQNLRHLSSMSNSFPVECLRENIAFELPQEFLQQTQPNKRDIKKAFYEMSLQAFNIFSQHTSKAWKERHLKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 106 - protein, 180 aa, Artificial
  synthetic
LDCNLLNVHLRRVTWQNLRHLSSMSNSFPVECLRENIAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRCLYYFYKFTALFRRK

SEQ ID NO: 107 - protein, 180 aa, Artificial
  synthetic
LDCNLLNVHLRRVTWQNLRHLSSMSNSFPVECLRENIAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 108 - protein, 180 aa, Artificial; identical to SEQ ID NO: 107
  synthetic

SEQ ID NO: 109 - protein, 180 aa, Artificial
  synthetic
LDCNLLNVHLRRVTWQNLRHLSSMSNSFPVECLRENIAFELPQEFLQYTQPMKRDIKKAFYEMSLQAFNIFSQHTFKYWKERHLKQIQTGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 110 - protein, 180 aa, Artificial
  synthetic
LDCNLLNVHLRRVTWQNLRHLSSMSNSFPVECLRENIAFELPQEFLQYTQPMKRDIKKAFYEMSLQAFNIFSQHTSKAWKERHLKQIQIGLDQQAEDLNQCLEEDENENEDMKEMKENEMKPSEARVPQLSSLELRRYFHRIDNFLKEKKYSDCAWEIVRAEIRRCLYYFYKFTALFRRK

SEQ ID NO: 111 - protein, 180 aa, Artificial
  synthetic
LDCNLLNVHLRRVTWQNLRHLSSMSNSFPVECLRENIAFELPQEFLQYTQPMKRDIKKAFYEMSLQAFNIFSQHTSKAWKERHLKQIQIGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 112 - protein, 180 aa, Artificial; identical to SEQ ID NO: 111
  synthetic

SEQ ID NO: 113 - protein, 180 aa, Artificial; identical to SEQ ID NO: 111
  synthetic

SEQ ID NO: 114 - protein, 180 aa, Artificial
  synthetic
LDCNLLNVHLRRVTWQNLRHLSSMSNSFPVECLRENIAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRAEIRRCLYYFYKFTALFRRK

SEQ ID NO: 115 - protein, 180 aa, Artificial
  synthetic
LDCNLLNVHLRRVTWQNLRHLSSMSNSFPVECLRENIAFELPQEFLQYTQPMKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 116 - protein, 180 aa, Artificial; identical to SEQ ID NO: 29
  synthetic

SEQ ID NO: 117 - protein, 180 aa, Artificial; identical to SEQ ID NO: 29
  synthetic

SEQ ID NO: 118 - protein, 180 aa, Artificial
  synthetic
LDCNLLNVHLRRVTWQNLRHLSSMSNSFPVECLRENNAFELPQEFLQQTQPNKRDIKKAFYEMSLQAFNIFSQHTSKAWKERHLKQIQTGLDQQAEDLNQCLEEDENENEDMKEMKENEMKPSEARVPQLSSLELRRYFHRIDNFLKEKKYSDCAWEIVRVEIRRCLYYFYKFTALFRRK

SEQ ID NO: 119 - protein, 180 aa, Artificial
  synthetic
LDCNLLNVHLRRVTWQNLRHLSSMSNSQPRECLRENNAFELPQEFLQYTQPMKRDIKKAFYEMSLQAFNIFSQHTSKAWKERHLKQIQIGLDQQAEDLNQCLEEDENENEDMKEMKENEMKPSEARVPQLSSLELRRYFHRIDNFLKEKKYSDCAWEIVRVEIRRALSYFTKFTALFRRK

SEQ ID NO: 120 - protein, 180 aa, Artificial; identical to SEQ ID NO: 27
  synthetic

SEQ ID NO: 121 - protein, 180 aa, Artificial; identical to SEQ ID NO: 28
  synthetic

SEQ ID NO: 122 - protein, 180 aa, Artificial; identical to SEQ ID NO: 30
  synthetic

SEQ ID NO: 123 - protein, 9 aa, Homo sapiens
YNLLGFLQR

SEQ ID NO: 124 - protein, 9 aa, Homo sapiens
LLGFLQRSS

SEQ ID NO: 125 - protein, 9 aa, Homo sapiens
FLQRSSNFQ

SEQ ID NO: 126 - protein, 9 aa, Homo sapiens
LQRSSNFQC

SEQ ID NO: 127 - protein, 9 aa, Homo sapiens
FQCQKLLWQ

SEQ ID NO: 128 - protein, 9 aa, Homo sapiens
WQLNGRLEY

SEQ ID NO: 129 - protein, 9 aa, Homo sapiens
YCLKDRMNF

SEQ ID NO: 130 - protein, 9 aa, Homo sapiens
MNFDIPEEI

SEQ ID NO: 131 - protein, 9 aa, Homo sapiens
LQQFQKEDA

SEQ ID NO: 132 - protein, 9 aa, Homo sapiens
LTIYEMLQN

SEQ ID NO: 133 - protein, 9 aa, Homo sapiens
YEMLQNIFA

SEQ ID NO: 134 - protein, 9 aa, Homo sapiens
LQNIFAIFR

SEQ ID NO: 135 - protein, 9 aa, Homo sapiens
FRQDSSSTG

SEQ ID NO: 136 - protein, 9 aa, Homo sapiens
WNETIVENL

SEQ ID NO: 137 - protein, 9 aa, Homo sapiens
INHLKTVLE

SEQ ID NO: 138 - protein, 9 aa, Homo sapiens
LKRYYGRIL

SEQ ID NO: 139 - protein, 9 aa, Homo sapiens
YYGRILHYL

SEQ ID NO: 140 - protein, 9 aa, Homo sapiens
ILHYLKAKE

SEQ ID NO: 141 - protein, 9 aa, Homo sapiens
LHYLKAKEY

SEQ ID NO: 142 - protein, 9 aa, Homo sapiens
WTIVRVEIL

SEQ ID NO: 143 - protein, 9 aa, Homo sapiens
IVRVEILRN

SEQ ID NO: 144 - protein, 9 aa, Homo sapiens
VRVEILRNF

SEQ ID NO: 145 - protein, 9 aa, Homo sapiens
VEILRNFYF

SEQ ID NO: 146 - protein, 9 aa, Homo sapiens
LRNFYFINR

SEQ ID NO: 147 - protein, 9 aa, Homo sapiens
FYFINRLTG

SEQ ID NO: 148 - protein, 9 aa, Homo sapiens
FINRLTGYL

SEQ ID NO: 149 - protein, 9 aa, Homo sapiens
INRLTGYLR

SEQ ID NO: 150 - protein, 9 aa, Artificial
  synthetic
WSLNGRLEY

SEQ ID NO: 151 - protein, 9 aa, Artificial
  synthetic
WNLNGRLEY

SEQ ID NO: 152 - protein, 9 aa, Artificial
  synthetic
WDLNGRLEY

SEQ ID NO: 153 - protein, 9 aa, Artificial
  synthetic
WELNGRLEY

SEQ ID NO: 154 - protein, 9 aa, Artificial
  synthetic
WHLNGRLEY

SEQ ID NO: 155 - protein, 9 aa, Artificial
  synthetic
WKVNGRLEY

SEQ ID NO: 156 - protein, 9 aa, Artificial
  synthetic
WQVNGRLEY

SEQ ID NO: 157 - protein, 9 aa, Artificial
  synthetic
WQFSGRLEY

SEQ ID NO: 158 - protein, 9 aa, Artificial
  synthetic
WQFTGRLEY

SEQ ID NO: 159 - protein, 9 aa, Artificial
  synthetic
WQFGGRLEY

SEQ ID NO: 160 - protein, 9 aa, Artificial
  synthetic
WQLSGRLEY

SEQ ID NO: 161 - protein, 9 aa, Artificial
  synthetic
WQLTGRLEY

SEQ ID NO: 162 - protein, 9 aa, Artificial
  synthetic
WQLGGRLEY

SEQ ID NO: 163 - protein, 9 aa, Artificial
  synthetic
WQLNSQLEY

SEQ ID NO: 164 - protein, 9 aa, Artificial
  synthetic
QSFDIPEEI

SEQ ID NO: 165 - protein, 9 aa, Artificial
  synthetic
QDFDIPEEI

SEQ ID NO: 166 - protein, 9 aa, Artificial
  synthetic
MSFDIPEEI

SEQ ID NO: 167 - protein, 9 aa, Artificial
  synthetic
MTFDIPEEI

SEQ ID NO: 168 - protein, 9 aa, Artificial
  synthetic
MGFDIPEEI

SEQ ID NO: 169 - protein, 9 aa, Artificial
  synthetic
MDFDIPEEI

SEQ ID NO: 170 - protein, 9 aa, Artificial
  synthetic
MEFDIPEEI

SEQ ID NO: 171 - protein, 9 aa, Artificial
  synthetic
MNYSIPEEI

SEQ ID NO: 172 - protein, 9 aa, Artificial
  synthetic
MNYNIPEEI

SEQ ID NO: 173 - protein, 9 aa, Artificial
  synthetic
MNYEIPEEI

SEQ ID NO: 174 - protein, 9 aa, Artificial
  synthetic
MNYQIPEEI

SEQ ID NO: 175 - protein, 9 aa, Artificial
  synthetic
MNFSIPEEI

SEQ ID NO: 176 - protein, 9 aa, Artificial
  synthetic
MNFNIPEEI

SEQ ID NO: 177 - protein, 9 aa, Artificial
  synthetic
MNFEIPEEI

SEQ ID NO: 178 - protein, 9 aa, Artificial
  synthetic
MNFQIPEEI

SEQ ID NO: 179 - protein, 9 aa, Artificial
  synthetic
MNFDIPESL

SEQ ID NO: 180 - protein, 9 aa, Artificial
  synthetic
MNFDIPESV

SEQ ID NO: 181 - protein, 9 aa, Artificial
  synthetic
MNFDIPENL

SEQ ID NO: 182 - protein, 9 aa, Artificial
  synthetic
MNFDIPENV

SEQ ID NO: 183 - protein, 9 aa, Artificial
  synthetic
MNFDIPEDL

SEQ ID NO: 184 - protein, 9 aa, Artificial
  synthetic
MNFDIPEDV

SEQ ID NO: 185 - protein, 9 aa, Artificial
  synthetic
MNFDIPEQL

SEQ ID NO: 186 - protein, 9 aa, Artificial
  synthetic
MNFDIPEQV

SEQ ID NO: 187 - protein, 9 aa, Artificial
  synthetic
MNFDIPEHL

SEQ ID NO: 188 - protein, 9 aa, Artificial
  synthetic
MNFDIPEHV

SEQ ID NO: 189 - protein, 9 aa, Artificial
  synthetic
MNFDIPERL

SEQ ID NO: 190 - protein, 9 aa, Artificial
  synthetic
MNFDIPERV

SEQ ID NO: 191 - protein, 9 aa, Artificial
  synthetic
MNFDIPEKL

SEQ ID NO: 192 - protein, 9 aa, Artificial
  synthetic
MNFDIPEKV

SEQ ID NO: 193 - protein, 9 aa, Artificial
  synthetic
MNFDIPEEL

SEQ ID NO: 194 - protein, 9 aa, Artificial
  synthetic
MNFDIPEEV

SEQ ID NO: 195 - protein, 9 aa, Artificial
  synthetic
HDMLQNIFA

SEQ ID NO: 196 - protein, 9 aa, Artificial
  synthetic
YSQLQNIFA

SEQ ID NO: 197 - protein, 9 aa, Artificial
  synthetic
YSLLQNIFA

SEQ ID NO: 198 - protein, 9 aa, Artificial
  synthetic
YSVLQNIFA

SEQ ID NO: 199 - protein, 9 aa, Artificial
  synthetic
YSFLQNIFA

SEQ ID NO: 200 - protein, 9 aa, Artificial
  synthetic
YEQLQNIFA

SEQ ID NO: 201 - protein, 9 aa, Artificial
  synthetic
YEMLQNIYT

SEQ ID NO: 202 - protein, 9 aa, Artificial
  synthetic
YEMLQNIWT

SEQ ID NO: 203 - protein, 9 aa, Artificial
  synthetic
YEMLQNIFT

SEQ ID NO: 204 - protein, 9 aa, Artificial
  synthetic
WSIVRVEIL

SEQ ID NO: 205 - protein, 9 aa, Artificial
  synthetic
WTIVRVSIL

SEQ ID NO: 206 - protein, 9 aa, Artificial
  synthetic
WTIVRVEMM

SEQ ID NO: 207 - protein, 9 aa, Artificial
  synthetic
WTIVRVEMV

SEQ ID NO: 208 - protein, 9 aa, Artificial
  synthetic
WTIVRVEMF

SEQ ID NO: 209 - protein, 9 aa, Artificial
  synthetic
WTIVRVELF

SEQ ID NO: 210 - protein, 9 aa, Artificial
  synthetic
WTIVRVEVF

SEQ ID NO: 211 - protein, 9 aa, Artificial
  synthetic
WTIVRVEFF

SEQ ID NO: 212 - protein, 9 aa, Artificial
  synthetic
WTIVRVEIM

SEQ ID NO: 213 - protein, 9 aa, Artificial
  synthetic
WTIVRVEIV

SEQ ID NO: 214 - protein, 9 aa, Artificial
  synthetic
WTIVRVEIF

SEQ ID NO: 215 - protein, 9 aa, Artificial
  synthetic
MNNFYFINR

SEQ ID NO: 216 - protein, 9 aa, Artificial
  synthetic
MENFYFINR

SEQ ID NO: 217 - protein, 9 aa, Artificial
  synthetic
MQNFYFINR

SEQ ID NO: 218 - protein, 9 aa, Artificial
  synthetic
MHNFYFINR

SEQ ID NO: 219 - protein, 9 aa, Artificial
  synthetic
MKNFYFINR

SEQ ID NO: 220 - protein, 9 aa, Artificial
  synthetic
LNNFYFINR

SEQ ID NO: 221 - protein, 9 aa, Artificial
  synthetic
LENFYFINR

SEQ ID NO: 222 - protein, 9 aa, Artificial
  synthetic
LQNFYFINR

SEQ ID NO: 223 - protein, 9 aa, Artificial
  synthetic
LHNFYFINR

SEQ ID NO: 224 - protein, 9 aa, Artificial
  synthetic
LKNFYFINR

SEQ ID NO: 225 - protein, 9 aa, Artificial
  synthetic
LRSFYFINR

SEQ ID NO: 226 - protein, 9 aa, Artificial
  synthetic
LRTFYFINR

SEQ ID NO: 227 - protein, 9 aa, Artificial
  synthetic
LRGFYFINR

SEQ ID NO: 228 - protein, 9 aa, Artificial
  synthetic
LRDFYFINR

SEQ ID NO: 229 - protein, 9 aa, Artificial
  synthetic
LREFYFINR

SEQ ID NO: 230 - protein, 9 aa, Artificial
  synthetic
LRQFYFINR

SEQ ID NO: 231 - protein, 9 aa, Artificial
  synthetic
LRHFYFINR

SEQ ID NO: 232 - protein, 9 aa, Artificial
  synthetic
LRKFYFINR

SEQ ID NO: 233 - protein, 9 aa, Artificial
  synthetic
LRNMYFINR

SEQ ID NO: 234 - protein, 9 aa, Artificial
  synthetic
LRNIYFINR

SEQ ID NO: 235 - protein, 9 aa, Artificial
  synthetic
LRNLYFINR

SEQ ID NO: 236 - protein, 9 aa, Artificial
  synthetic
LRNFHYVNR

SEQ ID NO: 237 - protein, 9 aa, Artificial
  synthetic
LRNFYFISQ

SEQ ID NO: 238 - protein, 9 aa, Artificial
  synthetic
LRNFYFISK

SEQ ID NO: 239 - protein, 9 aa, Artificial
  synthetic
LRNFYFITK

SEQ ID NO: 240 - protein, 9 aa, Artificial
  synthetic
LRNFYFIGK

SEQ ID NO: 241 - protein, 9 aa, Artificial
  synthetic
LRNFYFIDK

SEQ ID NO: 242 - protein, 9 aa, Artificial
  synthetic
LRNFYFIEK

SEQ ID NO: 243 - protein, 9 aa, Artificial
  synthetic
LRNFYFIQK

SEQ ID NO: 244 - protein, 9 aa, Artificial
  synthetic
LRNFYFIHK

SEQ ID NO: 245 - protein, 9 aa, Artificial
  synthetic
LRNFYFIRK

SEQ ID NO: 246 - protein, 9 aa, Artificial
  synthetic
LRNFYFIKK

SEQ ID NO: 247 - protein, 9 aa, Artificial
  synthetic
LRNFYFINE

SEQ ID NO: 248 - protein, 9 aa, Artificial
  synthetic
LRNFYFINQ

SEQ ID NO: 249 - protein, 9 aa, Artificial
  synthetic
LRNFYFINK

SEQ ID NO: 250 - protein, 9 aa, Artificial
  synthetic
FYFISQLTG

SEQ ID NO: 251 - protein, 9 aa, Artificial
  synthetic
MSYNQLGEL

SEQ ID NO: 252 - protein, 9 aa, Homo sapiens
MSYNLLGFL

SEQ ID NO: 253 - protein, 9 aa, Artificial
  synthetic
YNQLGELQR

SEQ ID NO: 254 - protein, 9 aa, Artificial
  synthetic
QLGELQRSS

SEQ ID NO: 255 - protein, 9 aa, Artificial
  synthetic
LGELQRSSN

SEQ ID NO: 256 - protein, 9 aa, Homo sapiens
LGFLQRSSN

SEQ ID NO: 257 - protein, 9 aa, Artificial
  synthetic
ELQRSSNFQ

SEQ ID NO: 258 - protein, 9 aa, Artificial
  synthetic
IPEEIKQKQ

SEQ ID NO: 259 - protein, 9 aa, Homo sapiens
IPEEIKQLQ

SEQ ID NO: 260 - protein, 9 aa, Artificial
  synthetic
IKQKQQFQK

SEQ ID NO: 261 - protein, 9 aa, Homo sapiens
IKQLQQFQK

SEQ ID NO: 262 - protein, 9 aa, Artificial
  synthetic
KQQFQKEDA

SEQ ID NO: 263 - protein, 9 aa, Artificial
  synthetic
LEKEDNTRG

SEQ ID NO: 264 - protein, 9 aa, Homo sapiens
LEKEDFTRG

SEQ ID NO: 265 - protein, 9 aa, Artificial
  synthetic
NTRGKEMSS

SEQ ID NO: 266 - protein, 9 aa, Homo sapiens
FTRGKLMSS

SEQ ID NO: 267 - protein, 9 aa, Artificial
  synthetic
EMSSRHLKR

SEQ ID NO: 268 - protein, 9 aa, Homo sapiens
LMSSLHLKR

SEQ ID NO: 269 - protein, 9 aa, Artificial
  synthetic
MSSRHLKRY

SEQ ID NO: 270 - protein, 9 aa, Homo sapiens
MSSLHLKRY

SEQ ID NO: 271 - protein, 9 aa, Artificial
  synthetic
RHLKRYYGR

SEQ ID NO: 272 - protein, 9 aa, Homo sapiens
LHLKRYYGR

SEQ ID NO: 273 - DNA, 840 bp, Homo sapiens
acattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 274 - protein, 20 aa, Artificial
  synthetic
MGSSHHHHHHSSGLVPRGSH

(19) United States
(12) Patent Application Publication
(10) Pub. No.: US 2005/0054053 A1
(43) Pub. Date: Mar. 10, 2005

(54) Interferon variants with improved properties

(75) Inventors: Anna Marie Aguinaldo, Altadena, CA (US); Amelia Joy Beyna, Cheverly, MD (US); Ho Sung Cho, Monrovia, CA (US); John Rudolph Desjarlais, Pasadena, CA (US); Shannon Alicia Marshall, San Francisco, CA (US); Umesh Muchhal, Monrovia, CA (US); Michael Francis Aquino Villegas, Los Angeles, CA (US); Eugene Zhukovsky, West Hollywood, CA (US); Michael Stephen Quesenberry, Los Angeles, CA (US)

Correspondence Address: Robin M. Silva, Esq.;Dorsey & Whitney LLP, Intellectual Property Department, Four Embarcadero Center, Suite 3400, San Francisco, CA, 94111-4187, US

(73) Assignee: Xencor, Inc.

(21) Appl. No.: 10/820,467

(22) Filed: Mar. 30, 2004

Related U.S. Application Data
(63) Continuation-in-part of application No. 10/676,705, filed on Sep. 30, 2003.
(60) Provisional application No. 60/489,725, filed on Jul. 24, 2003.
(60) Provisional application No. 60/477,246, filed on Jun. 10, 2003.
(60) Provisional application No. 60/415,541, filed on Oct. 1, 2002.

(51) Int. Cl. C07H 21/04; C12P 21/04; C07K 14/565

(52) U.S. Cl. 435/69.51; 435/320.1; 435/325; 530/351; 536/23.5

(57) ABSTRACT

The invention relates to interferon variants with improved properties and methods for their use.

This application claims benefit of priority under 35 USC 119(e)(1) to U.S. Ser. No. : 60/415,541, filed Oct. 1, 2002; U.S. Ser. No. : 60/477,246, filed Jun. 10, 2003, and 60/489,725, filed Jul. 24, 2003, and 10/676,705, filed Sep. 30, 2003, all hereby incorporated by reference in their entirety.

### FIELD OF THE INVENTION

The invention relates to variants of type I interferons with improved properties, and to methods of making and to methods and compositions utilizing these variants. the use of a variety of computational methods, including Protein Design Automation® (PDA®) technology, to identify interferon variants with improved properties, generate computationally prescreened secondary libraries of proteins, and to methods of making and methods and compositions utilizing soluble variants and the libraries.

### BACKGROUND OF THE INVENTION

Interferons (IFNs) are a well-known family of cytokines secreted by a large variety of eukaryotic cells. A possessing a range of biological activities is associated with interferons including antiviral, anti-proliferative, neoplastic and immunoregulatory immunomodulatory activities. Interferons have demonstrated utility in the treatment of a variety of diseases, and are in widespread use for the treatment of multiple sclerosis and viral hepatitis; the most common therapeutic applications are currently treatment of hepatitis C and multiple sclerosis.

Interferons include a number of related proteins, such as interferon-alpha (IFN-α), interferon-beta (IFN-β), interferon-gamma IFN-γ) interferon-kappa (IFN-κ, also known as interferon-epsilon or IFN-ε), interferon-tau (IFN-τ), and interferon-omega (IFN-ω). These interferon proteins are produced in a variety of cell types: IFN-α (leukocytes), IFN-β (fibroblasts), IFN-g (lymphocytes), IFN-ε or κ (keratinocytes), IFN-ω (leukocytes) and IFN-τ (trophoblasts). IFN-α, IFN-β, IFN-ε or κ, IFN-ω, and IFN-τ are classified as type I interferons, while IFN-γ is classified as a type II interferon. Interferon alpha is encoded by a multi-gene family, while the other interferons appear to each be coded by a single gene in the human genome. Furthermore, there is some allelic variation in interferon sequences among different members of the human population.

Type-I interferons all appear to bind a common receptor, type I IFN-R, composed of IFNAR1 and IFNAR2 subunits. The exact binding mode and downstream signal transduction cascades differ somewhat among the type I interferons. However, in general, the JAK/STAT signal transduction pathway is activated following binding of interferon to the interferon receptor. STAT transcription factors then translocate to the nucleus, leading to the expression of a number of proteins with antiviral, antineoplastic, and immunomodulatory activities.

The properties of naturally occurring type I interferon proteins are not optimal for therapeutic use. Type I interferons induce injection site reactions and a number of other side effects. They are highly immunogenic, eliciting neutralizing and non-neutralizing antibodies in a significant fraction of patients. IInterferons are poorly absorbed from the subcutaneous injection site and have short serum half-lives. Finally, type I interferons do not express solubly in prokaryotic hosts, thus necessitating more costly and difficult refolding or mammalian expression protocols.

The present invention is directed to identification of interferon proteins with improved properties. A number of groups have generated modified interferons with improved properties; the references below are all expressly incorporated by reference in their entirety.

Cysteine-depleted variants have been generated to minimize formation of unwanted inter- or intra-molecular disulfide bonds (U.S. Pat. Nos. 4,518,584;, 4,588,585;, 4,959,314). Methionine-depleted variants have been generated to minimize susceptibility to oxidation (EPO 260350).

Interferons with modified activity have been generated (U.S. Pat. Nos. 6,514,729; 4,738,844; 4,738,845; 4,753,795; 4,766,106; WO 00/78266). U.S. Pat. Nos. 5,545,723 and 6,127,332 disclose substitution mutants of interferon beta at position 101. Chimeric interferons comprising sequences from one or more interferons have been made (Chang et. al. Nature Biotech. 17: 793-797 (1999), U.S. Pat. Nos. 4,758,428; 4,885,166; 5,382,657; 5,738,846). Substitution mutations to interferon beta at positions 49 and 51 have also been described (U.S. Pat. No. 6,531,122).

Interferon beta variants with enhanced stability have been claimed, in which the hydrophobic core was optimized using rational design methods (WO 00/68387). Alternate formulations that promote interferon stability or solubility have also been disclosed (U.S. Pat. Nos. 4,675,483; 5,730,969; 5,766,582; WO 02/38170).

Interferon beta muteins with enhanced solubility have been claimed, in which several leucine and phenylalanine residues are replaced with serine, threonine, or tyrosine residues (WO 98/48018). However, it is not clear whether any of the variants claimed are sufficiently soluble, stable, and active to constitute improved variants.

Interferon alpha and interferon beta variants with reduced immunogenicity have been claimed (See WO 02/085941 and WO 02/074783). However, due to the large number of variants disclosed and the apparent lack of consideration of the structural and functional effects of the introduced mutations, one skilled in the art faces a problem in identifying a variant that would be a functional, less immunogenic interferon variant suitable for administration to patients may be difficult.

Immunogenicity is a major limitation of current interferon (including interferon beta) therapeutics. Although immune responses are typically most severe for non-human proteins, even therapeutics based on human proteins, such as interferon beta, are often observed to be immunogenic. Immunogenicity is a complex series of responses to a substance that is perceived as foreign and may include production of neutralizing and non-neutralizing antibodies, formation of immune complexes, complement activation, mast cell activation, inflammation, and anaphylaxis.

Several factors can contribute to protein immunogenicity, including but not limited to the protein sequence, the route and frequency of administration, and the patient population. Aggregation has been linked to the immunogenicity of a related protein therapeutic, interferon alpha [Braun et. al. Pharm. Res. 1997 14: 1472-1478]. Another study suggests that the presence of DR15 MHC alleles increases susceptibility to neutralizing antibody formation; interestingly, the same alleles also confer susceptibility to multiple sclerosis [Stickler et. al. Genes Immun. 2004 5: 1-7].

As aggregation may contribute to the immunogenicity of interferons (particularly interferon beta), variants engineered for improved solubility may also possess reduced immunogenicity. Cysteine-depleted variants have been generated to minimize formation of unwanted inter- or intra-molecular disulfide bonds (U.S. Pat. Nos. 4,518,584; 4,588,585; 4,959,314); such variants show a reduced propensity for aggregation. Interferon beta variants with enhanced stability have been claimed, in which the hydrophobic core was optimized using rational design methods (WO 00/68387); in some cases solubility may be enhanced by improvements in stability. Alternate formulations that promote interferon stability and solubility have also been disclosed (U.S. Pat Nos. 4,675,483; 5,730,969; 5,766,582; WO 02/38170). Interferon beta muteins with enhanced solubility have been claimed, in which several leucine and phenylalanine residues are replaced with serine, threonine, or tyrosine residues (WO 98/48018). However, it is not clear whether any of the variants claimed are sufficiently soluble, stable, and active to constitute improved variants.

Interferons have been modified by the addition of polyethylene glycol (“PEG”) (see U.S. Pat. Nos. 4,917,888; 5,382,657; WO 99/55377; WO 02/09766; WO 02/3114). PEG addition can improve serum half-life and solubility. In some cases, PEGylation has been observed to reduce the fraction of patients who raise neutralizing antibodies by sterically blocking access to antibody agretopes (see for example, Hershfield et. al. PNAS 1991 88:7185-7189 (1991); Bailon. et al. Bioconjug. Chem. 12: 195-202(2001); He et al. Life Sci. 65: 355-368 (1999)).

Interferon beta variants have also been generated that are predicted to bind class II MHC alleles with decreased affinity relative to the wild type protein; in both examples primarily alanine mutations were used to confer decreased binding [WO 02/074783; Stickler supra].

Several methods have been developed to modulate the immunogenicity of proteins; a preferred approach is to disrupt T-cell activation by removing MHC-binding agretopes. This approach is more tractable than evading T-cell receptor or antibody binding, as the diversity of MHC molecules comprises only ˜10³ alleles, while the antibody repertoire is estimated to be approximately 10⁸ and the T-cell receptor repertoire is larger still. By identifying and removing or modifying class II MHC-binding peptides within a protein sequence, the molecular basis of immunogenicity can be evaded. The elimination of such agretopes for the purpose of generating less immunogenic proteins has been disclosed previously; see for example WO 98/52976, WO 02/079232, and WO 00/3317.

While a large number of mutations in MHC-binding agretopes may be identified that are predicted to confer reduced immunogenicity, most of these amino acid substitutions will be energetically unfavorable. As a result, the vast majority of the reduced immunogenicity sequences identified using the methods described above will be incompatible with the structure and/or function of the protein. In order for MHC agretope removal to be a viable approach for reducing immunogenicity, it is crucial that simultaneous efforts are made to maintain a protein's structure, stability, and biological activity.

Immunogenicity may limit the efficacy and safety of interferon therapeutics in multiple ways. Therapeutic efficacy may be reduced directly by the formation of neutralizing antibodies. Efficacy may also be reduced indirectly, as binding to either neutralizing or non-neutralizing antibodies may alter serum half-life. Unwanted immune responses may take the form of injection site reactions, including but not limited to delayed-type hypersensitivity reactions. It is also possible that anti-interferon beta neutralizing antibodies may cross-react with endogenous interferon beta and block its function.

There remains a need for novel interferon proteins having reduced immunogenicity. Variants of interferon with reduced immunogenicity could find use in the treatment of a number of interferon responsive conditions.

As a result, there exists a need for the development and discovery of interferon proteins with improved properties, including but not limited to increased efficacy, decreased side effects, decreased immunogenicity, increased solubility, and enhanced soluble prokaryotic expression. Improved interferon therapeutics could may be useful for the treatment of a variety of diseases and conditions, including autoimmune diseases, viral infections, and, inflammatory diseases, cell proliferation diseases, bacterial infections, enhancing fertility, and cancer, among others and transplant rejection. In addition, interferons may be used to promote the establishment of pregnancy in certain mammals.

### SUMMARY OF THE INVENTION

The present invention is related to variants of type I human interferons with at least one improved property, including but not limited to increased solubility (for example by an inability to multimerize, particularly upon administration), increased specific activity, and modified immunogenicity.

In one aspect, the invention provides variant type 1 interferon beta (IFN-β) proteins exhibiting modified immunogenicity as compared to a wild type (IFN-β). A number of wild-type interferons of use in the present invention are shown in SEQ ID NOS: 1-18. Modified immunogenicity includes reduced immunogenicity, for example where the variant protein demonstrates reduced binding to at least one human class II MHC allele, or when the variant exhibits improved solubility. Increased solubility can be obtained by substituting at least one solvent-exposed hydrophobic residue. Modified immunogenicity also includes increased immunogenicity.

The invention also provides variant interferons that exhibit modified immunogenicity while substantially maintaining interferon biological activity, including, but not limited to, immunomodulatory activity, antiviral activity and antineoplastic activities.

In an additional aspect, the invention provides IFN-β variants exhibiting modified immunogenicity comprising at least one modification at a position selected from the group consisting of 1,2, 3, 4, 5, 6, 8, 9, 12, 15, 16, 22, 28, 30, 32, 36, 42, 43, 46, 47, 48, 49, 51, 92, 93, 96, 100, 101, 104, 111, 113, 116, 117, 120, 121, 124,130, 148, and 155. In some cases, the modifications to residues 5, 8, 15, 47, 111, 116, and 120 are substitution mutations preferably selected from the group consisting of alanine, arginine, aspartic acid, asparagine, glutamic acid, glutamine, glycine, histidine, and lysine. Similarly, the modifications to residues 22, 28, 30, 32, 36, 92, 130, 148, and 155 are preferably selected from the group including alanine, arginine, aspartic acid, asparagine, glutamic acid, glutamine, glycine, histidine, serine, threonine and lysine. These variants are particularly preferred for increased solubility leading to reduced immunogenicity.

Particularly preferred variant IFN-β proteins comprise at least one modification selected from the group consisting of:L5A, L5D, L5E, L5K, L5N, L5Q, L5R, L5S, L5T, F8A, F8D, F8E, F8K, F8N, F8Q, F8R, F8S, S12E, S12K, S12Q, S12R, W22E, L28Q, Y30H, L32A, E43K, E43R, L47K, Y92Q, E104R, E104K, E104H, E104Q, E104A, F111N, R113D, R113E, R113Q, R113A, L116D, L116E, L116N, L116Q, L116R, M117R, L120D, L120R, L130R, V148A, and Y155S. Particularly preferred variants have the sequences outlined in SEQ ID NO:19 (variant 2), SEQ ID NO:20 (variant 7), SEQ ID NO:21 (variant 15), SEQ ID NO:22 (variant 23), SEQ ID NO:23 (variant 36), SEQ ID NO:24 (variant 39) and SEQ ID NO:25 (variant 64).

In a further aspect, variant IFN-β, proteins with reduced immunogenicity exhibit reduced binding at least one human class II MHC allele. In this embodiment (as well as for other modified immunogenicity variants) at least one amino acid modification is made in at least one of the following positions: agretope 1: residues 3-11; agretope 2: residues 5-13; agretope 3: residues 8-16; agretope 4: residues 9-17; agretope 5: residues 15-23; agretope 6: residues 22-30; agretope 7: residues 30-38; agretope 8: residues 36-44; agretope 9: residues 47-55; agretope 10: residues 57-65; agretope 11: residues 60-68; agretope 12: residues 63-71; agretope 13: residues 70-78; agretope 14: residues 79-87; agretope 15: residues 95-103; agretope 16: residues 122-130; agretope 17: residues 125-133; agretope 18: residues 129-137; agretope 19: residues 130-138; agretope 20: residues 143-151; agretope 21: residues 145-153; agretope 22: residues 146-154; agretope 23: residues 148-156; agretope 24: residues 151-159; agretope 25: residues 154-162; agretope 26: residues 156-164; agretope 27: residues 157-165. Preferred variants also include amino acid modifications in 2 or more of these agretopes.

Preferred variants in agretope 6 include SEQ ID NOS:**1-14. Preferred variants in agretope 8 include SEQ ID NOS:**15-45. Preferred variants in agretope 11 include SEQ ID NOS:**46-54. Preferred variants in agretope 20 include SEQ ID NOS:**55-65. Preferred variants in agretope 24 include SEQ ID NOS:**66-100. A preferred variants in agretope 25 includes SEQ ID NO: **101.

In a further aspect, the invention provides variant type 1 interferon alpha (IFN-α) proteins exhibiting modified immunogenicity as compared to a wild type IFN-α comprising at least one modification at a position selected from the group consisting of 16, 27, 30, 89, 100, 110, 111, 117, 128, and 161. In a preferred embodiment, the modifications are substitution mutations selected from the group consisting of alanine, arginine, aspartic acid, asparagine, glutamic acid, glutamine, glycine, histidine, serine, threonine, and lysine. In a preferred embodiment, the variants exhibit enhanced solubility.

In an additional aspect, the invention provides variant type 1 interferon kappa (IFN-κ) proteins exhibiting modified immunogenicity as compared to a wild type IFN-κ comprising at least one modification at a position selected from the group consisting of 1, 5, 8, 15, 18, 28, 30, 33, 37, 46, 48, 52, 65, 68, 76, 79, 89, 97, 112, 115, 120,127, 133, 151, 161, 168, and 171. Preferred substitutions are selected from the group consisting of alanine, arginine, aspartic acid, asparagine, glutamic acid, glutamine, glycine, histidine, serine, threonine, and lysine. In a preferred embodiment, the variants exhibit enhanced solubility.

Particularly preferred IFN-κ variants comprise at least one modification selected from the group consisting of L5Q, V8N, W15R, F28Q, F28S, V30R, 137N, Y48Q, M52N, M52Q, F76S, Y78A, 189T, Y97D, M112T, M115G, L133Q, V161A, C166A, Y168S, and Y171T.

In a further aspect, the invention provides recombinant nucleic acids encoding the variant proteins, expression vectors containing the variant nucleic acids, host cells comprising the variant nucleic acids and/or expression vectors, and methods for producing the variant proteins.

In an additional aspect, the invention provides treating an interferon responsive disorder by administering to a patient a variant protein, usually with a pharmaceutical carrier, in a therapeutically effective amount.

In a further aspect, the invention provides methods for modulating immunogenicity (particularly reducing immunogenicity) of interferons (particularly IFN-β) by altering MHC Class II epitopes.

### BRIEF DESCRIPTION OF THE DRAWINGS

FIG. 1 shows amino acid sequences for human type I interferons and some preferred variants SEQ ID NOS:1-30.

FIG. 2 shows a sequence alignment of human interferon-alpha subtypes, SEQ ID NOs:31-43.

FIG. 3 shows the sequence alignment of IFN-a2a (1ITF), IFN-b (1AU1), IFN-k (IFNK), and IFN-t (1B5L) (SEQ ID NOs:44-47) that was used to construct the homology model of interferon-kappa.

FIG. 4 shows ISRE assay dose-response curves for interferon beta variants.

FIG. 5 shows a dot blot assay used to test for soluble expression of interferon-kappa variants. G12 and H12 are positive controls, whereas E12 and F12 are soluble extracts from cells expressing WT interferon-kappa (negative control). Wells C5, C8, D4, E5 and F2 represent clones expressing soluble interferon-kappa variants.

FIG. 6 shows a dot blot assay used to test for soluble expression of interferon-kappa variants. G12 and H12 are positive controls, whereas E12 and F12 are soluble extracts from cells expressing WT interferon-kappa (negative control). Most of the putative soluble clones test positive (soluble expression) upon reexpression.

FIG. 7 shows a western blot of solubly expressed interferon kappa variants. The arrow indicates the expected position of interferon-kappa protein. Lanes 2 and 3 are total soluble fraction from WT interferon-kappa expressing cells, respectively. Lanes 4-15 are soluble fractions from the lysates of different variants. exposed hydrophobic residues in interferon-kappa and suitable polar replacements as determined by PDA® technology calculations.

FIG. 8 shows exposed hydrophobic residues in interferon-kappa and suitable polar replacements according to sequence alignment data.

FIG. 9 depicts a method for engineering less immunogenic interferon derivatives, particularly IFN-β.

FIG. 10 depicts a schematic representation of a method for in vitro testing of the immunogenicity of interferon peptides or proteins with IVV technology, particularly IFN-β.

FIG. 11 graphically shows decreased aggregation of SEQ ID NO: 20 variant as compared to BetaSeron® (Schering AG/Berlex) at pH 3.0 over time (9 h at 37° C.). The top graph shows XENP806, the middle graph shows BetaSeron and the bottom graph shows wild type IFNB.

FIG. 12 graphically shows decreased aggregation of SEQ ID NO: 20 variant as compared to BetaSeron® (Schering AG/Berlex) at 6 pH 6.0±1.0 over time (9 h at 37° C.). The top graph shows XENP806, the middle graph shows BetaSeron and the bottom graph shows wild type IFNB.

### DETAILED DESCRIPTION OF THE INVENTION

Definitions: By “control sequences” and grammatical equivalents herein is meant nucleic acid sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acids are “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, elements such as enhancers do not have to be contiguous.

By “immunogenicity” and grammatical equivalents herein is meant the ability of a protein to elicit an immune response, including but not limited to production of neutralizing and non-neutralizing antibodies, formation of immune complexes, complement activation, mast cell activation, inflammation, and anaphylaxis.

By “reduced immunogenicity” and grammatical equivalents herein is meant a decreased ability to activate the immune system, when compared to the wild type protein. For example, an IFN variant protein can be said to have “reduced immunogenicity” if it elicits neutralizing or non-neutralizing antibodies in lower titer or in fewer patients than wild type IFN. In a preferred embodiment, the amount of neutralizing antibodies is decreased by at least 5%, with at least 50% or 90% decreases being especially preferred. Therefore, if a wild type produces an immune response in 10% of patients, a variant with reduced immunogenicity would produce an immune response in not more than 9.5% of patients, with less than 5% or less than 1% being especially preferred. An IFN variant protein is also said to have “reduced immunogenicity” if it shows decreased binding to one or more MHC alleles or if it induces T-cell activation in a decreased fraction of patients relative to wild type IFN. In a preferred embodiment, the probability of T-cell activation is decreased by at least 5%, with at least 50% or 90% decreases being especially preferred.

By “interferon aggregates” and grammatical equivalents herein is meant protein-protein complexes comprising at least one interferon molecule (and often multiple IFN molecules) and possessing less bioactivity as compared to the wild-type or parent molecule, including immunomodulatory, antiviral, or antineoplastic activity than the corresponding monomeric interferon molecule. Interferon aggregates include interferon dimers, interferon-albumin dimers, higher order species, etc.

By “interferon-responsive disorders” and grammatical equivalents herein is meant diseases, disorders, and conditions that can benefit from treatment with a type I interferon. Examples of interferon-responsive disorders include, but are not limited to, autoimmune diseases (e.g. multiple sclerosis, diabetes mellitus, lupus erythematosus, Crohn's disease, rheumatoid arthritis, stomatitis, asthma, allergies and psoriasis), infectious diseases including viral infections (hepatitis C, papilloma viruses, hepatitis B, herpes viruses, viral encephalitis, cytomegalovirus, and rhinovirus), and cell proliferation diseases including cancer (e.g. osteosarcoma, basal cell carcinoma, cervical dysplasia, glioma, acute myeloid leukemia, multiple myeloma, chronic lymphocytic leukemia, Kaposi's sarcoma, chronic myelogenous leukemia, renal-cell carcinoma, ovarian cancers, hairy-cell leukemia, and Hodgkin's disease). Interferons may also be used to promote the establishment of pregnancy in certain mammals, and to reduce transplant rejection.

By “library” as used herein is meant a collection of protein sequences that are likely to take on a particular fold or have particular protein properties. The library preferably comprises a set of sequences resulting from computation, which may include energy calculations or statistical or knowledge based approaches. Libraries that range in size from about 50 to about 10¹³ sequences are preferred. Libraries are generally generated experimentally and analyzed for the presence of members possessing desired protein properties.

By “modification” and grammatical equivalents is meant insertions, deletions, or substitutions to a protein or nucleic acid sequence, with substitutions being preferred.

By “naturally occurring” or “wild -type” or “wt” and grammatical equivalents thereof herein is meant an amino acid sequence or a nucleotide sequence that is found in nature and includes allelic variations. In a preferred embodiment, the wild-type sequence is the most prevalent human sequence. In addition, in most cases the modifications of amino acids of the invention are in relation to a wild-type “starting” molecule. However, as will be appreciated by those in the art, other starting molecules, generally referred to herein as “parents”, are already non-naturally occurring IFN derivatives that are further modified using the methods of the invention. While human sequences are generally preferred, in some embodiments the wild type IFN proteins may be from any number of organisms, include, but are not limited to, rodents (rats, mice, hamsters, guinea pigs, etc.), primates, and farm animals (including sheep, goats, pigs, cows, horses, etc).

By “nucleic acid” and grammatical equivalents herein is meant DNA, RNA, or molecules, which contain both deoxy- and ribonucleotides. Nucleic acids include genomic DNA, cDNA and oligonucleotides including sense and anti-sense nucleic acids. Nucleic acids may also contain modifications, such as modifications in the ribose-phosphate backbone that confer increased stability and half-life.

A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human.

“Pharmaceutically acceptable carrier” or grammatical equivalents includes pharmaceutically acceptable carriers, excipients or stabilizers (Remington's Pharmaceutical Sciences 16th edition, Osol, A. Ed., 1980), in the form of lyophilized formulations, aqueous solutions, etc. Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG). In a preferred embodiment, the pharmaceutical composition that comprises the compositions of the present invention is in a water-soluble form, such as being present as pharmaceutically acceptable salts, which is meant to include both acid and base addition salts. Acceptable carriers include, but are not limited to pharmaceutically acceptable acid and base salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The following residues are defined herein to be “polar” residues: aspartic acid, asparagine, glutamic acid, glutamine, lysine, arginine, histidine, serine, and threonine. In some embodiments, as is further outlined below, these residues are particularly useful to replace hydrophobic residues on the surface of proteins to avoid aggregation.

By “protein” herein is meant a molecule comprising at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids ([see Simon et al., Proc. Natl. Acad. Sci. U.S.A. 89(20:9367-71 (1992))]. For example, homo-phenylalanine, citrulline, and noreleucine are considered amino acids for the purposes of the invention. “Amino acid” also includes amino acid residues such as proline and hydroxyproline. B, and both D- and L- amino acids may be utilized.

By “protein properties” herein is meant biological, chemical, and physical properties including but not limited to enzymatic activity, specificity (including substrate specificity, kinetic association and dissociation rates, reaction mechanism, and pH profile), stability (including thermal stability, stability as a function of pH or solution conditions, resistance or susceptibility to ubiquitination or proteolytic degradation), solubility, aggregation, structural integrity, crystallizability, binding affinity and specificity (to one or more molecules including proteins, nucleic acids, polysaccharides, lipids, and small molecules), oligomerization state, dynamic properties (including conformational changes, allostery, correlated motions, flexibility, rigidity, folding rate), subcellular localization, ability to be secreted, ability to be displayed on the surface of a cell, posttranslational modification (including N- or C-linked glycosylation, lipidation, and phosphorylation), ammenability to synthetic modification (including PEGylation, attachment to other molecules or surfaces), and ability to induce altered phenotype or changed physiology (including cytotoxic activity, immunogenicity, toxicity, ability to signal, ability to stimulate or inhibit cell proliferation, ability to induce apoptosis, and ability to treat disease). When a biological activity is the property, modulation in this context includes both an increase or a decrease in activity.

By “solubility” and grammatical equivalents herein is meant the maximum possible concentration of monomeric protein in a solution of specified condition. By “soluble expression” and grammatical equivalents herein is meant that the protein is able to be produced at least partially in soluble form rather than in inclusion bodies when expressed in a prokaryotic host. It is preferred that at least 1 mg soluble protein is produced per 100 mL culture, with at least 10 mg or 100 mg being especially preferred. By “improved solubility” and grammatical equivalents herein is meant an increase in the maximum possible concentration of monomeric protein in solution. Preferred solutions in this context include the pharmaceutically acceptable carrier, as well as the site of administration. For example, if the naturally occurring protein can be concentrated to 1 mM and the variant can be concentrated to 5 mM under the same solution conditions, the variant can be said to have improved solubility. In a preferred embodiment, solubility is increased by at least a factor of 2, with increases of at least 5× or 10× being especially preferred. As will be appreciated by those skilled in the art, solubility is a function of solution conditions. For the purposes of this invention, solubility should be assessed under solution conditions that are pharmaceutically acceptable. Specifically, pH should be between 6.0 and 8.0, salt concentration should be between 50 and 250 mM. Additional buffer components such as excipients may also be included, although it is preferred that albumin is not required.

By “therapeutically effective dose” herein is meant a dose that produces the effects for which it is administered. The exact dose will depend on the purpose of the treatment, and will be ascertainable by one skilled in the art using known techniques. In a preferred embodiment, dosages of about 5 μg/kg are used, administered either intravenously or subcutaneously. As is known in the art, adjustments for variant IFN protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

By “treatment” herein is meant to include therapeutic treatment, as well as prophylactic, or suppressive measures for the disease or disorder. Thus, for example, successful administration of a variant IFN protein prior to onset of the disease may result in treatment of the disease. As another example, successful administration of a variant IFN protein after clinical manifestation of the disease to combat the symptoms of the disease comprises “treatment” of the disease. “Treatment” also encompasses administration of a variant IFN protein after the appearance of the disease in order to ameliorate or eradicate the disease. Successful administration of an agent after onset and after clinical symptoms have developed, with possible abatement of clinical symptoms and perhaps amelioration of the disease, further comprises “treatment” of the disease.

By “variant IFN interferon nucleic acids” and grammatical equivalents herein is meant nucleic acids that encode variant IFN interferon proteins. Due to the degeneracy of the genetic code, an extremely large number of nucleic acids may be made, all of which encode the variant IFN proteins of the present invention, by simply modifying the sequence of one or more codons in a way which that does not change the amino acid sequence of the variant IFN.

By “variant IFN proteins” or “non-naturally occurring IFN interferon proteins” and grammatical equivalents thereof herein is meant non-naturally occurring IFN proteins which differ from the wild type IFN protein by at least one (1) amino acid insertion, deletion, or substitution. It should be noted that unless otherwise stated, all positional numbering of variant IFN proteins and variant IFN nucleic acids is based on the wild-type sequences. IFN variants are characterized by the predetermined nature of the variation, a feature that sets them apart from naturally occurring allelic or interspecies variation of the IFN protein sequence. The IFN variants must typically either exhibit the same qualitative biological activity as the naturally occurring IFN or have been specifically engineered to have alternate biological properties. Preferably the IFN variants of the present invention retain at least 50% of at least one wild type interferon activity, as determined using the ISRE assay described below. Variants that retain at least 75% or 90% of wild type activity are more preferred, and variants that are more active than wild type are especially preferred. The variant interferon IFN proteins may contain insertions, deletions, and/or substitutions at the N-terminus, C-terminus, or internally. In a preferred embodiment, variant IFN proteins have at least 1 residue that differs from the most similar human IFN sequence, with at least 2, 3, 4, or 5 different residues being more preferred. Variant IFN proteins may contain further modifications, for instance mutations that alter solubility or additional protein properties such as stability or immunogenicity or which enable or prevent posttranslational modifications such as PEGylation or glycosylation. Variant IFN interferon proteins may be subjected to co- or post-translational modifications, including but not limited to synthetic derivatization of one or more side chains or termini, glycosylation, PEGylation, circular permutation, cyclization, fusion to proteins or protein domains, and addition of peptide tags or labels.

By “9-mer peptide frame” and grammatical equivalents herein is meant a linear sequence of nine amino acids that is located in a protein of interest. 9-mer frames may be analyzed for their propensity to bind one or more class II MHC alleles as is further described below.

By “allele” and grammatical equivalents herein is meant an alternative form of a gene. Specifically, in the context of class II MHC molecules, alleles comprise all naturally occurring sequence variants of DRA, DRB1, DRB3/4/5, DQA1, DQB1, DPA1, and DPB1 molecules.

By “hit” and grammatical equivalents herein is meant, in the context of the matrix method, that a given peptide is predicted to bind to a given class II MHC allele. In a preferred embodiment, a hit is defined to be a peptide with binding affinity among the top 5%, or 3%, or 1% of binding scores of random peptide sequences. In an alternate embodiment, a hit is defined to be a peptide with a binding affinity that exceeds some threshold, for instance a peptide that is predicted to bind an MHC allele with at least 100 μM or 10 μM or 1 μM affinity.

By “matrix method” and grammatical equivalents thereof herein is meant a method for calculating peptide—MHC affinity in which a matrix is used that contains a score for each possible residue at each position in the peptide, interacting with a given MHC allele. The binding score for a given peptide—MHC interaction is obtained by summing the matrix values for the amino acids observed at each position in the peptide.

By “MHC-binding agretopes” and grammatical equivalents herein is meant peptides that are capable of binding to one or more class II MHC alieles with appropriate affinity to enable the formation of MHC—peptide—T-cell receptor complexes and subsequent T-cell activation. MHC-binding agretopes are linear peptide sequences that comprise at least approximately 9 residues.

Naturally occurring interferons possess antiviral, antiproliferative, and immunomodulatory activities, making interferons valuable therapeutics. However, drugs based on naturally occurring interferons suffer from a number of liabilities, including a high incidence of side effects and immunogenicity.

Accordingly, the present invention provides novel variants of type I interferon proteins. These interferon variants comprise one or more modifications that were selected to improve biophysical properties and clinical performance. In general, two main properties are altered as described herein. In a preferred embodiment, solubility is altered. Poor solubility contributes to many of the liabilities of current interferon therapeutics. Accordingly, a primary focus of this invention is interferon variants with improved solubility. Secondly, as further described below, IFNs can be engineered to alter the binding of the IFN to human class II MHC alleles, particularly exhibiting reduced binding.

Although type I interferons are biologically active as monomers, they are known to form dimers and higher order species. These species can consist primarily of interferon proteins, or may also contain additional proteins such as human serum albumin. Non-monomeric interferon species exhibit significantly decreased activity, as even dimer formation interferes with receptor binding (Utsumi et. al. Biochim. Biophys. Acta 998: 167 (1989) and Runkel et. al. Pharm. Res. 15: 641 (1998)). Interferon therapeutics are known to elicit neutralizing antibodies in a substantial fraction of patients (Antonelli et. al. Eur. Cytokine Netw. 10: 413 (1999)). Poor solubility may be a significant contributing factor to the immunogenicity of interferon therapeutics, as aggregates are typically more immunogenic than soluble proteins (Speidel et. al. Eur. J. Immunol. 27: 2391 (1997)), and aggregation has been demonstrated to increase the immunogenicity of interferon-alpha (Braun et. al. Pharm. Res. 14: 1472 (1997)). Furthermore, poor solubility results in reduced absorption following subcutaneous injection (Clodfelter et. al. Pharm. Res. 15: 254 (1998)).

Protein solubility is essential for the discovery, manufacturing, and clinical utilization of protein therapeutics. Protein solubility comprises two features: soluble expression and resistance to aggregation. Poor solubility hinders structural and functional characterization and drives up production costs by necessitating refolding or mammalian expression. In protein therapeutics, aggregates exhibit decreased efficacy due to intrinsic inactivity of aggregated species (see Utsumi et. al. Biochim. Biophys. Acta 998: 167 (1989) and Runkel et. al. Pharm. Res. 15: 641 (1998)) and reduced absorption following subcutaneous injection (Clodfelter et. al. Pharm. Res. 15: 254 (1998)). To compensate, larger doses can be used, although this often causes side effects. Aggregates are also far more likely to raise an immune response than soluble proteins (Braun et. al. Pharm. Res. 14: 1472 (1997), Speidel et. al. Eur. J. Immunol. 27: 2391 (1997)).

Poor solubility contributes to many of the liabilities of current interferon therapeutics. Although type I interferons are biologically active as monomers, they are known to form dimers and higher order species. Non-monomeric interferon species exhibit significantly decreased activity, as even dimer formation interferes with receptor binding (Utsumi, Runkel supra). Interferon therapeutics are known to elicit neutralizing antibodies in a substantial fraction of patients (Antonelli et. al. Eur. Cytokine Netw. 10: 413 (1999)). Aggregates are typically more immunogenic than soluble proteins, and aggregation has been demonstrated to increase the immunogenicity of interferon-alpha (Braun, supra).

A variety of strategies may be utilized to design IFN variants with improved solubility. In a preferred embodiment, one or more of the following strategies are used: 1) reduce hydrophobicity by substituting one or more solvent-exposed hydrophobic residues with suitable polar residues, 2) increase polar character by substituting one or more neutral polar residues with charged polar residues, 3) decrease formation of intermolecular disulfide bonds by modifying one or more non-disulfide bonded cysteine residues (unpaired cysteines) with suitable non-cysteine residues, and 4) reduce the occurrence of known unwanted protein-protein interactions by modifying one or more residues located at protein-protein interaction sites such as dimer interfaces with alternate residues with a decreased propensity to form protein-protein interactions. As will be appreciated by those in the art, several alternative strategies in combination are also contemplated. For example, modifications that, 5) increase protein stability, for example by one or more modifications that improve packing in the hydrophobic core, improve helix capping and dipole interactions, or remove unfavorable electrostatic interactions, and 6) modify one or more residues that can affect the isoelectric point of the protein (that is, aspartic acid, glutamic acid, histidine, lysine, arginine, tyrosine, and cysteine residues) to decrease the isoelectric point of the protein below physiological pH. Increasing the stability of a protein can improve solubility by decreasing the population of partially folded or misfolded states. Additionally, protein solubility is typically at a minimum when the isoelectric point of the protein is equal to the pH of the surrounding solution. Modifications that perturb the isoelectric point of the protein away from the pH of a relevant environment, such as serum, may therefore serve to improve solubility. Furthermore, modifications that decrease the isoelectric point of a protein can improve injection site absorption (Holash et. al. PNAS 99: 11393-11398 (2002)).

Type I interferons typically have one free cysteine residue and several exposed hydrophobic residues. These positions can be targeted for mutagenesis in order to improve solubility. Replacing exposed hydrophobic residues with appropriate polar residues can also decrease the number of MHC-binding epitopes. (See U.S. Ser. No. 10/039,170, filed Jan. 8, 2003, hereby incorporated by reference). Since MHC binding is a key step in the initiation of an immune response, such mutations may decrease immunogenicity by multiple mechanisms.

In two cases, type I interferons have been observed to crystallize as dimers or higher order species. While the dimeric structure is not biologically significantly less active than the monomer, it may represent an inactive species that is present in interferon therapeutics. Accordingly, residues located at or close to the protein-protein interfaces can be targeted for modification.

The present invention is directed to identification of interferon proteins with improved properties, see U.S. Pat. Nos. 6, 188,965; 6,269,312; 6,403,312; 6,433,145; U.S. Ser. No. : 60/368,014; PCT/00/13216; WO9848018A1; WO00/05371 and WO01/07608, all expressly incorporated by reference in their entirety.

A number of methods can be used to identify modifications (that is, insertion, deletion, or substitution mutations) that will yield interferon variants with improved solubility and while retaining or improving other bioactivities, including but not limited to immunomodulatory, antiviral, or antineoplastic activity. These include, but are not limited to, sequence profiling (Bowie and Eisenberg, Science 253(5016): 164-70, (1991)), rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994)); and residue pair potentials (Jones, Protein Science 3: 567-574, (1994)).

In an especially preferred embodiment, rational design of improved IFN variants is achieved by using Protein Design Automation® (PDA®) technology. (See U.S. Pat. Nos. 6,188,965; 6,269,312; 6,403,312; WO98/47089 and U.S. Ser. Nos. 09/058,459, 09/127,926, 60/104,612, 60/158,700, 09/419,351, 60/181,630, 60/186,904, 09/419,351, 09/782,004 and 09/927,790, 60/347,772, and 10/218,102; and PCT/US01/218,102 and U.S. Ser. No. 10/218,102, U.S. Ser. No. 60/345,805; U.S. Ser. No. 60/373,453 and U.S. Ser. No. 60/374,035, all references expressly incorporated herein in their entirety.)

PDA® technology couples computational design algorithms that generate quality sequence diversity with experimental high-throughput screening to discover proteins with improved properties. The computational component uses atomic level scoring functions, side chain rotamer sampling, and advanced optimization methods to accurately capture the relationships between protein sequence, structure, and function. Calculations begin with the three-dimensional structure of the protein and a strategy to optimize one or more properties of the protein. PDA® technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 10⁵⁰ sequences or more, and quickly return a tractable number of sequences that are predicted to satisfy the design criteria. Useful modes of the technology span from combinatorial sequence design to prioritized selection of optimal single site substitutions. PDA® technology has been applied to numerous systems including important pharmaceutical and industrial proteins and has a demonstrated record of success in protein optimization.

In a preferred embodiment, each polar residue is represented using a set of discrete low-energy side-chain conformations (see for example Dunbrack Curr. Opin. Struct. Biol. 12:431-440 (2002)). A preferred force field may include terms describing van der Waals interactions, hydrogen bonds, electrostatic interactions, and salvation, among others.

In a preferred embodiment, preferred suitable polar residues are defined as those polar residues: 1) Whose energy in the optimal rotameric configuration is more favorable than the energy of the exposed hydrophobic residue at that position and 2) Whose energy in the optimal rotameric configuration is among the most favorable of the set of energies of all polar residues at that position

In a preferred embodiment, Dead-End Elimination (DEE) is used to identify the rotamer for each polar residue that has the most favorable energy (see Gordon et. al. J. Comput Chem. 24: 232-243 (2003), Goldstein Biophys. J. 66: 1335-1340 (1994) and Lasters and Desmet, Prot. Eng. 6: 717-722 (1993)).

In an alternate embodiment, Monte Carlo can be used in conjunction with DEE to identify groups of polar residues that have favorable energies.

In an alternate preferred embodiment, a sequence prediction algorithm (SPA) is used to design proteins that are compatible with a known protein backbone structure as is described in Raha, K., et al. (2000) Protein Sci., 9: 1106-1119; U.S. Ser. No. 09/877,695, filed Jun. 8, 2001 and 10/071,859, filed Feb. 6, 2002.

In a preferred embodiment, after performing one or more computational calculations, including PDA® technology calculations, a library of variant proteins is designed, experimentally constructed, and screened for desired properties.

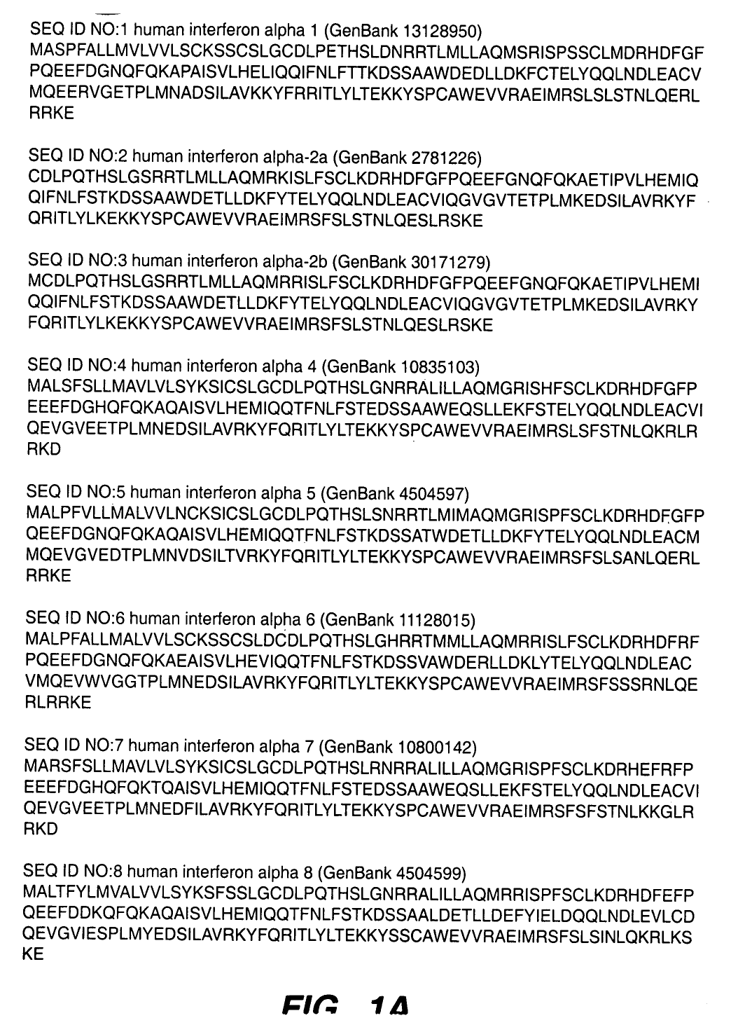

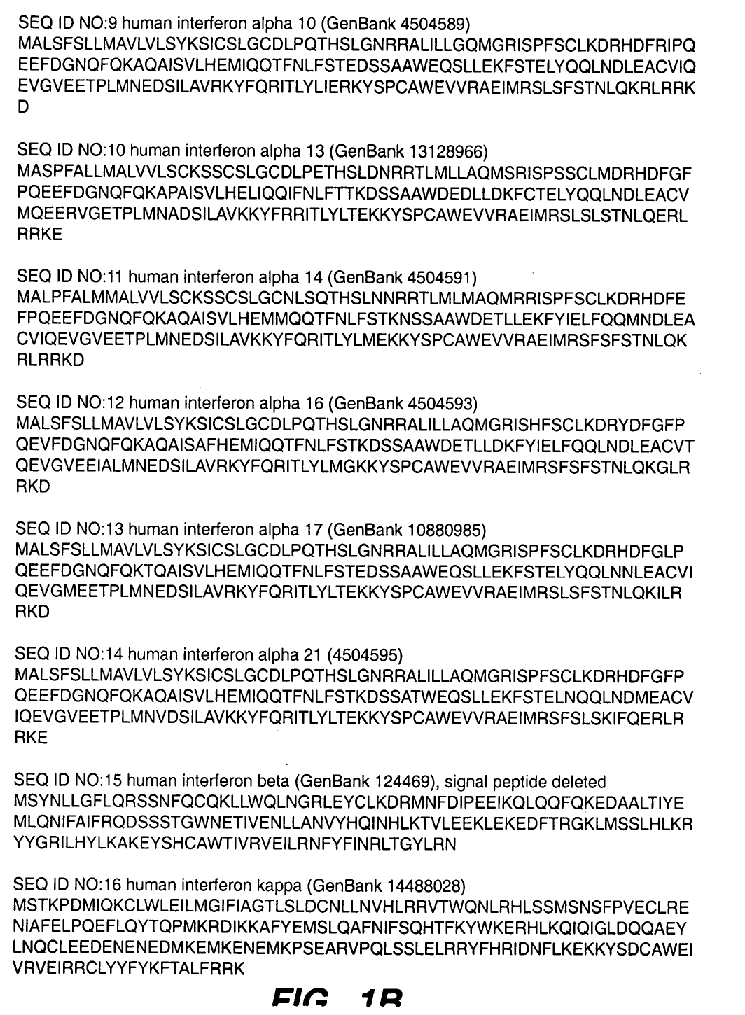

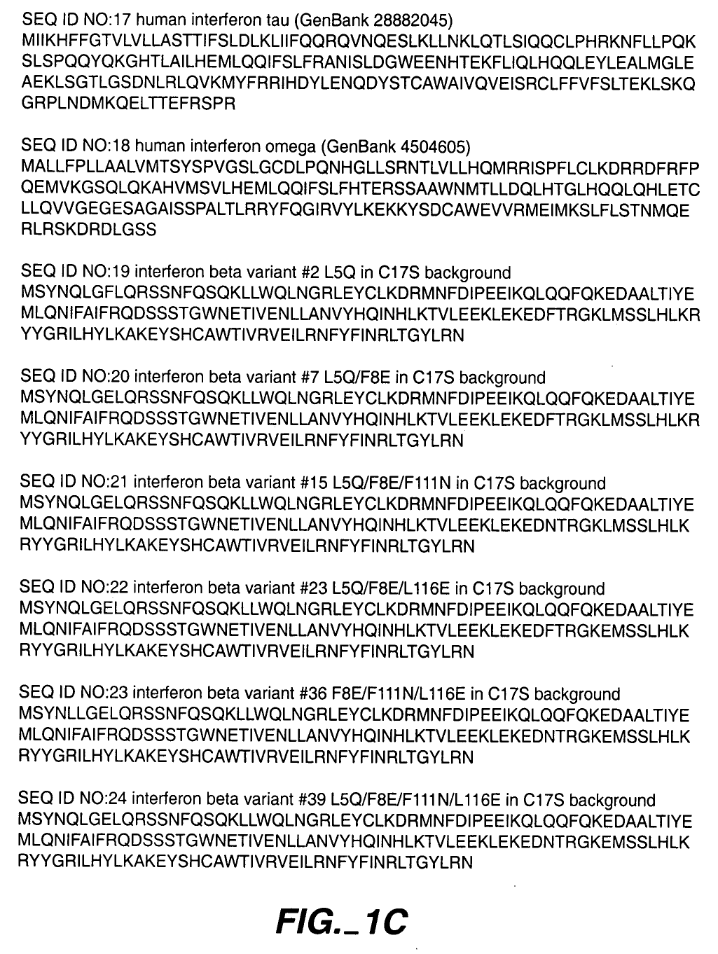

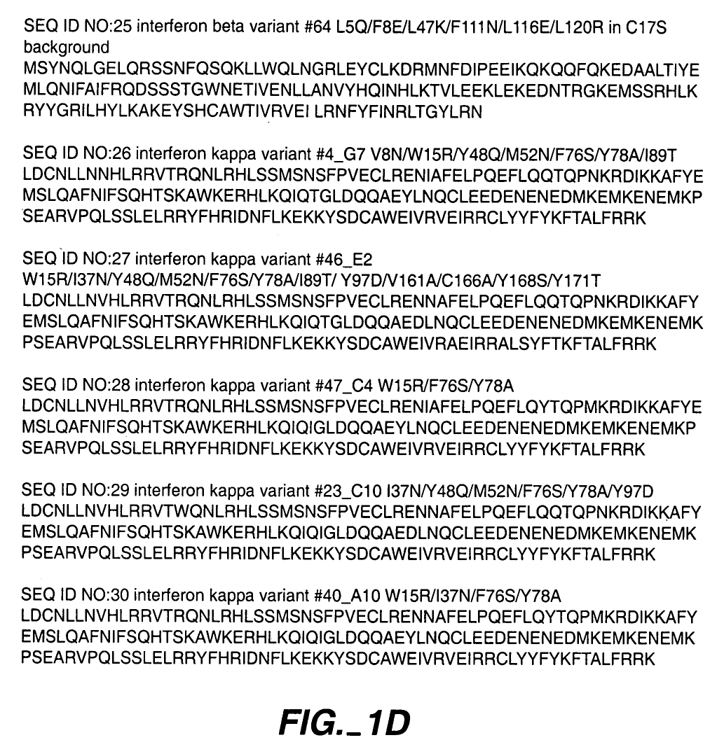

In one embodiment, the library is a combinatorial library, meaning that the library comprises all possible combinations of allowed residues at each of the variable positions. For example, if positions 3 and 9 are allowed to vary, allowed choices at position 3 are A, V, and I, and allowed choices at position 9 are E and Q, the library includes the following sequences: 3A/9E, 3A/9Q, 3V/9E, 3V/9Q, 3I/9E, and 3I/9Q. In a preferred embodiment, PDA® technology calculations may be used to modify wild type interferon sequences to generate novel, non-naturally occurring, soluble proteins from known interferon sequences (see for example, FIG. 1). See U.S. Pat. Nos. 6,188,965; 6,269,312; 6,403,312, expressly incorporated by reference herein.

In a preferred embodiment, the polar residues that are included in the library at each variable position are deemed suitable by both PDA® technology calculations and by sequence alignment data. Alternatively, one or more of the polar residues that are included in the library are deemed suitable by either PDA® technology calculations or sequence alignment data.

In a preferred embodiment, residues that are close in sequence are “coupled” in the library, meaning that all combinatorial possibilities are not sampled. For instance, if the library includes residues L and Q at position 5 and residues F and E at position 8, a “coupled” library could include L5/F8 and Q5/E8 but not include L5/E8 or Q5/F8. Coupling residues decreases the overall combinatorial complexity of the library, thereby simplifying screening. Furthermore, coupling can be used to avoid the introduction of two or more modifications that are incompatible with each other. For interferon kappa, preferred suitable residues based on both PDA® calculations and sequence alignment data are described herein. In an alternate embodiment, the polar residues that are included in the library at each variable position are deemed suitable by either PDA® technology calculations or by sequence alignment data or by both methods. Preferred suitable residues based on both PDA® calculations and sequence alignment data are shown in FIG. 2.

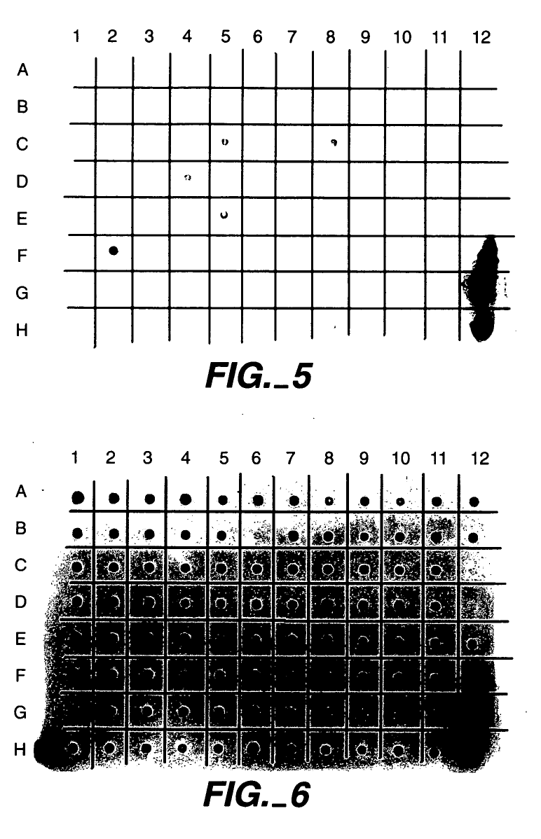

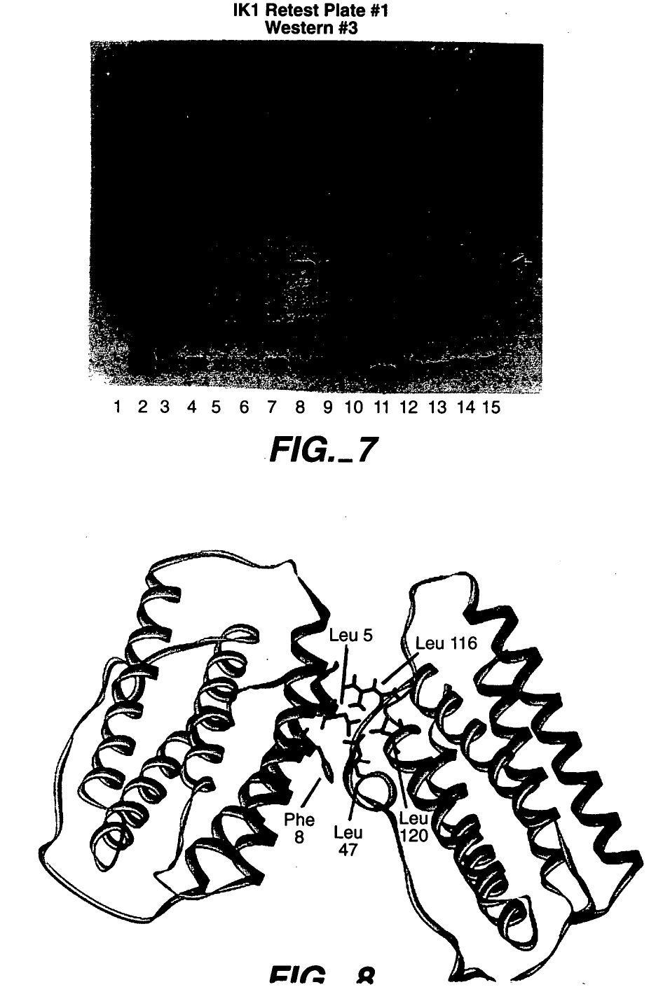

Preferred, suitable residues based on both PDA® technology calculations and sequence alignment data are shown in FIG. 5.

Obtaining structures of type I interferons PDA® technology calculations, described above, require a template protein structure. In the a most preferred embodiment, the structure of a type I interferon is obtained by solving its crystal structure or NMR structure by techniques well known in the art. High- resolution structures are available for type I interferons including interferon-a2a (interferon-alpha2a), interferon -a2b (interferon-alpha2b), interferon-b (interferon-beta), and interferon-t (interferon-tau) (see Radhakrishnan et. al. J. Mol. Biol. 286:151-162 (1999), Karpusas et. al. Proc. Nat. Acad. Sci. USA 94:22 (1997), Klaus et. al. J. Mol. Biol. 274:661-675 (1997), Radhakrishnan et. al. Structure 4:1453-1463 (1996)).

In an alternate embodiment, a homology model is built, using methods known to those in the art. Homology models of interferons have been constructed earlier: See for example Seto et. al. Protein Sci. 4:655-670 (1995). The homology model may be derived from one or more of the high resolution structures listed above.

In a preferred embodiment, the BLAST alignment algorithm is used to generate alignments proteins that are homologs of an interferon of interest. Examples of homologous proteins include other classes of type I interferons, allelic variants of interferon, and interferons from other species.

Identifying solvent-exposed hydrophobic residue positions. Hydrophobic residues as used herein generally are identified as valine, leucine, isoleucine, methionine, phenylalanine, tyrosine, and tryptophan. Exposed residues as used herein as those residues whose side chains have at least 30 Å² (square Angstroms) of solvent accessible surface area. As will be appreciated by those skilled in the art, other values such as 50 Å² or fractional values such as 50% could be used instead. Furthermore, alternative methods such as contact models, among others, may be used to identify exposed residues.

Identifying suitable polar residues for each exposed hydrophobic position. In a preferred embodiment, solvent exposed hydrophobic residues are replaced with structurally and functionally compatible polar residues. As used herein, polar residues generally include serine, threonine, histidine, aspartic acid, asparagine, glutamic acid, glutamine, arginine, and lysine. Alanine and glycine may also serve as suitable replacements, constituting a reduction in hydrophobicity. Solvent exposed hydrophobic residues can be defined according to absolute or fractional solvent accessibility, as defined above. It is also possible to use other methods, such as contact models, to identify exposed residues.

In a preferred embodiment, suitable polar residues include alanine, serine, threonine, histidine, aspartic acid, asparagine, glutamic acid, glutamine, arginine, and lysine.

In a preferred embodiment, suitable polar residues include only the subset of polar residues that are observed in analogous positions in homologous proteins, especially other interferons.

In an especially preferred embodiment, suitable polar residues include only the subset of polar residues with low or favorable energies as determined using PDA® technology or SPA™ calculations.

As used herein, for example, solvent exposed hydrophobic residues in interferon-alpha 2a include, but are not limited to, Met 16, Phe 27, Leu 30, Tyr 89, IIe 100, Leu 110, Met 111, Leu 117, Leu 128, and Leu 161.

Especially preferred solvent exposed hydrophobic residues are those that have not been implicated in interferon alpha function or receptor binding (see for example Piehler et. al. J. Biol. Chem. 275: 40425-40433 (2000), Hu et. al. J. Immunol. 163: 854-860 (1999), Hu et. al. J. Immunol. 167: 1482-1489 (2001)), including Met 16, Phe 27, IIe 100, Leu 110, Met 111, Leu 117, and Leu 161.

Especially preferred modifications to interferon-alpha include, but are not limited to, M16D, F27Q, I100Q, L110N, M111Q, L117R, and L161E.

As used herein, for example, solvent exposed hydrophobic residues in interferon-beta include, but are not limited to, Leu 5, Phe 8, Phe 15, Trp 22, Leu 28, Tyr 30, Leu 32, Met 36, Leu 47, Tyr 92, Phe 111, Leu 116, Leu 120, Leu 130, Val 148, and Tyr 155.

Especially preferred modifications to interferon-beta include, but are not limited to, L5Q, F8E, F111N, L116E, and L120R.

Especially preferred solvent exposed hydrophobic residues are those residues that have not been implicated in interferon beta function or receptor binding (see for example Runkel et. al. Biochem. 39: 2538-2551 (2000), Runkel et. al. J. Int. Cytokine Res. 21: 931-941 (2001)), include Leu 5, Phe 8, Leu 47, Phe 111, Leu 116, and Leu 120.

As used herein, for example, solvent exposed hydrophobic residues in interferon-kappa include, but are not limited to, Leu 1, Leu 5, Val 8, Trp 15, Leu 18, Phe 28, Val 30, Leu 33, IIe 37, Leu 46, Tyr 48, Met 52, Leu 65, Phe 68, Phe 76, Tyr 78, Trp 79, IIe 89, Tyr 97, Met 112, Met 115, Met 120, Val 127, Leu 133, Tyr 151, Val 161, Tyr 168, and Tyr 171.

Especially preferred solvent exposed hydrophobic residues are located at positions that are polar in other interferon sequences, and include Leu 5, Val 8, Trp 15, Phe 28, Val 30, IIe 37, Tyr 48, Met 52, Phe 76, Tyr 78, IIe 89, Tyr 97, Val 161, Tyr 168, and Tyr 171.

Especially preferred modifications to interferon-kappa include, but are not limited to, L5Q, V8N, W15R, F28Q, V30R, 137N, Y48Q, M52N, F76S, Y78A, 189T, Y97D, M112T, M115G, L133Q, V161A, Y168S, and Y171T.

Identifying unpaired cysteine positions. Unpaired cysteines are defined to be cysteines that do not form a disulfide bond in the folded protein. Unpaired cysteines can be identified, for example, by visual analysis of the structure or by analysis of the disulfide bond patterns of related proteins.

Interferon alpha-1 and interferon alpha-13 contain one unpaired cysteine at position 86 (Cys 86).

Interferon-beta contains one unpaired cysteine at position 17 (Cys 17).

Interferon-kappa contains one unpaired cysteine at position 166 (Cys 166).

Ovine interferon-tau contains one unpaired cysteine at position 86 (Cys 86).

Identifying suitable non-cysteine residues for each unpaired cysteine position. Suitable non-cysteine residues as used herein are meant all amino acid residues other than cysteine.

In a preferred embodiment, if the cysteine position is substantially buried in the protein core, suitable non-cysteine residues include alanine and the hydrophobic residues valine, leucine, isoleucine, methionine, phenylalanine, tyrosine, and tryptophan.

In a preferred embodiment, if the cysteine position is substantially exposed to solvent, suitable non-cysteine residues include alanine and the polar residues serine, threonine, histidine, aspartic acid, asparagine, glutamic acid, glutamine, arginine, and lysine.

In a preferred embodiment, suitable residues are defined as those with low (favorable) energies as calculated using PDA® technology.

In a preferred embodiment, suitable residues defined as those that are observed at homologous positions in proteins that are interferon-kappa homologs. For example, position 86 is an unpaired cysteine in some interferon-alphal and interferon-alphal3, but is replaced with tyrosine or serine in other interferon alpha subtypes. Also, position 166 is an unpaired cysteine in interferon-kappa, but is frequently alanine in other interferon sequences.

In a more preferred embodiment, suitable residues are those, which that have both low (favorable) energies as calculated using PDA® technology and are observed at homologous positions in proteins that are interferon-kappa homologsin the analogous position in other interferon proteins.

In a most preferred embodiment, Cys 86 in interferon-alpha 1 or interferon alpha-13 replaced by glutamic acid, lysine, or glutamine.

In a most preferred embodiment, Cys 17 in interferon-beta is replaced by alanine, aspartic acid, asparagine, serine or threonine.

In a most preferred embodiment, Cys 166 in interferon-kappa is replaced by Alanine, glutamic acid, or histidine.

In some embodiments, the variant INF proteins of the invention do not include substitutions at unpaired cysteine postions.

Identifying dimer interface residues. In a preferred embodiment, residues that mediate intermolecular interactions between interferon monomers or between interferon and human serum albumin are replaced with structurally and functionally compatible residues that confer decreased propensity for unwanted intermolecular interactions.

In a preferred embodiment, interface residues are defined as those residues located within 8 Å of a protein-protein contact. Distances of less than 5 Å are especially preferred. Distances may be measured using any structure, with high- resolution crystal structures being especially preferred.

Preferred interface residues in interferon alpha include, but are not limited to, residues 16, 19, 20, 25, 27, 28, 30, 33, 35-37, 39-41, 44-46, 54, 58, 61, 65, 68, 85, 91, 99, 112-115, 117, 118, 121, 122, 125, and 149.

Preferred interface residues in interferon beta include, but are not limited to, residues 1-6, 8, 9, 12, 16, 42, 43, 46, 47,49, 51, 93, 96, 97, 100, 101, 104, 113, 116, 117, 120, 121, and 124.

Identifying suitable residues for each interface position. Suitable residues for interface residues as used herein are meant all amino acid residues that are compatible with the structure and function of a type I interferon, but which are substantially incapable of forming unwanted intermolecular interactions, including but not limited to interactions with other interferon molecules and interactions with human serum albumin.

Typically, the interface positions will be substantially exposed to solvent. In such cases, preferred substitutions include alanine and the polar residues serine, threonine, histidine, aspartic acid, asparagine, glutamic acid, glutamine, arginine, and lysine. However, for interface positions that are substantially buried in the monomer structure, hydrophobic replacements are preferred.

In a preferred embodiment, suitable polar residues include only the subset of polar residues that are observed in analogous positions in homologous proteins, especially other interferons, that do not form a given unwanted intermolecular interaction.

In an especially preferred embodiment, suitable polar residues include only the subset of polar residues with low or favorable energies as determined using PDA® technology calculations or SPA calculations (described above).

In a most especially preferred embodiment, suitable polar residues include only the subset of polar residues that are determined to be compatible with the monomer structure and incompatible with a given unwanted intermolecular interaction, as determined using PDA® technology calculations or SPA calculations.

Especially preferred modifications to interferon-beta include L5A, L5D, L5E, L5K, L5N, L5Q, L5R, L5S, L5T, F8A, F8D, F8E, F8K, F8N, F8Q, F8R, F8S, S12E, S12K, S12Q, S12R, E43K, E43R, R113D, L116D, L116E, L116N, L116Q, L116R, and M117R.

In a preferred embodiment, each polar residue is represented using a set of discrete low-energy side-chain conformations obtained from the 1996 Dunbrak and Karplus rotamer library. A preferred force field may include terms describing van der Waals interactions, hydrogen bonds, electrostatic interactions, and solvation, among others.

In a preferred embodiment, DEE is used to identify the rotamer for each polar residue that has the most favorable energy.

In an alternate embodiment, Monte Carlo can be used in conjunction with DEE to identify groups of polar residues that have favorable energies.

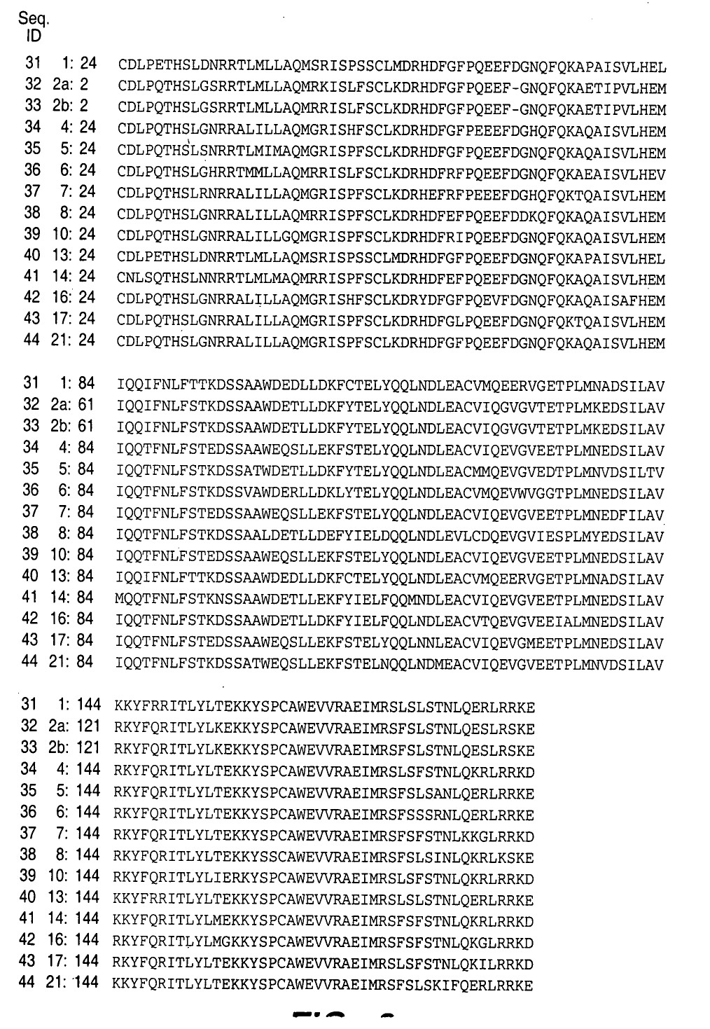

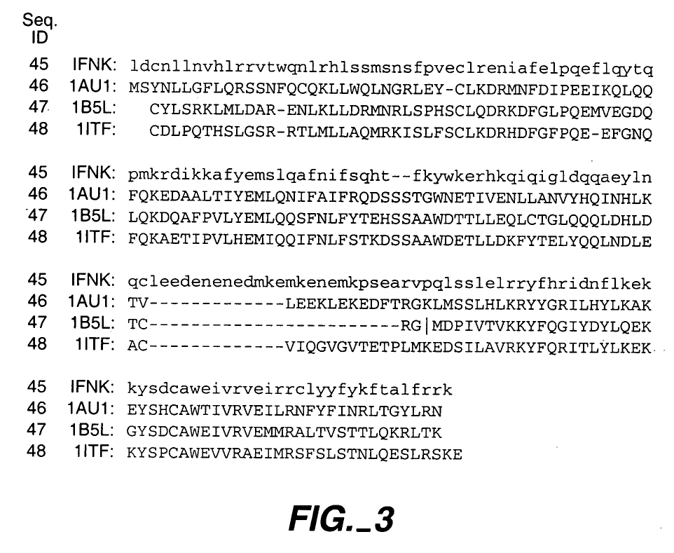

In a preferred embodiment, the frequency of occurrence of each polar residue at each position in interferon-kappa homologs is normalized using the method of Henikoff & Henikoff (J. Mol. Biol. 243: 547-578 (1994)). In an alternate embodiment, a simple count of the number of occurrences of each polar residue at each position is made. For interferon kappa, preferred suitable residues based on sequence alignment data include those shown in Figure x.Preferred suitable residues based on sequence alignment data include those shown in FIG. 3.

Especially preferred modifications to interferon-alpha include, but are not limited to, M16D, F27Q, I100Q, L110N, M111Q, L117R, and L161E.

Additional preferred, suitable residues are shown in FIG. 6. These residues are located at positions that are close in primary sequence to the residues in FIG. 5 and are suitable based on either PDA™ technology calculations or sequence alignment data. Diversity at such positions can be incorporated into a library of protein variants without increasing the total combinatorial complexity of the library.

Identification of MHC-binding agretopes in interferons The following discussion is centered on INFβ. Although as will be appreciated by those in the art, these techniques apply to the other IFNs as well. MHC-binding peptides are obtained from proteins by a process called antigen processing. First, the protein is transported into an antigen presenting cell (APC) by endocytosis or phagocytosis. A variety of proteolytic enzymes then cleave the protein into a number of peptides. These peptides can then be loaded onto class II MHC molecules, and the resulting peptide-MHC complexes are transported to the cell surface. Relatively stable peptide-MHC complexes can be recognized by T-cell receptors that are present on the surface of naïve T cells. This recognition event is required for the initiation of an immune response. Accordingly, blocking the formation of stable peptide-MHC complexes is an effective approach for preventing unwanted immune responses.

The factors that determine the affinity of peptide-MHC interactions have been characterized using biochemical and structural methods. Peptides bind in an extended conformation bind along a groove in the class II MHC molecule. While peptides that bind class II MHC molecules are typically approximately 13-18 residues long, a nine-residue region is responsible for most of the binding affinity and specificity. The peptide binding groove can be subdivided into “pockets”, commonly named P1 through P9, where each pocket is comprises the set of MHC residues that interacts with a specific residue in the peptide. A number of polymorphic residues face into the peptide-binding groove of the MHC molecule. The identity of the residues lining each of the peptide-binding pockets of each MHC molecule determines its peptide binding specificity. Conversely, the sequence of a peptide determines its affinity for each MHC allele.

Several methods of identifying MHC-binding agretopes in protein sequences are known in the art and may be used to identify agretopes in type I interferons, including IFN-β.

Sequence-based information can be used to determine a binding score for a given peptide-MHC interaction (see for example Mallios, Bioinformatics 15: 432-439 (1999); Mallios, Bioinformatics 17: p942-948 (2001); Sturniolo et. al. Nature Biotech. 17: 555-561(1999)). It is possible to use structure-based methods in which a given peptide is computationally placed in the peptide-binding groove of a given MHC molecule and the interaction energy is determined (for example, see WO 98/59244 and WO 02/069232). Expressly incorporated herein by reference. Such methods may be referred to as “threading” methods. Alternatively, purely experimental methods can be used; for example a set of overlapping peptides derived from the protein of interest can be experimentally tested for the ability to induce T-cell activation and/or other aspects of an immune response. (see for example WO 02/77187).

In a preferred embodiment, MHC-binding propensity scores are calculated for each 9-residue frame along the interferon beta sequence using a matrix method (see Sturniolo et. al., supra; Marshall et al, J. Immunol. 154: 5927-5933 (1995), and Hammer et al., J. Exp. Med. 180: 2353-2358 (1994)). Expressly incorporated herein by reference. It is also possible to consider scores for only a subset of these residues, or to consider also the identities of the peptide residues before and after the 9-residue frame of interest. The matrix comprises binding scores for specific amino acids interacting with the peptide binding pockets in different human class II MHC molecule. In the most preferred embodiment, the scores in the matrix are obtained from experimental peptide binding studies. In an alternate preferred embodiment, scores for a given amino acid binding to a given pocket are extrapolated from experimentally characterized alleles to additional alleles with identical or similar residues lining that pocket. Matrices that are produced by extrapolation are referred to as “virtual matrices”.

In a preferred embodiment, the matrix method is used to calculate scores for each peptide of interest binding to each allele of interest. Several methods can then be used to determine whether a given peptide will bind with significant affinity to a given MHC allele. In one embodiment, the binding score for the peptide of interest is compared with the binding propensity scores of a large set of reference peptides. Peptides whose binding propensity scores are large compared to the reference peptides are likely to bind MHC and may be classified as “hits”. For example, if the binding propensity score is among the highest 1% of possible binding scores for that allele, it may be scored as a “hit” at the 1% threshold. The total number of hits at one or more threshold values is calculated for each peptide. In some cases, the binding score may directly correspond with a predicted binding affinity. Then, a hit may be defined as a peptide predicted to bind with at least 100 μM or 10 μM or 1 μM affinity.

In a preferred embodiment, the number of hits for each 9-mer frame in the protein is calculated using one or more threshold values ranging from 0.5% to 10%. In an especially preferred embodiment, the number of hits is calculated using 1%, 3%, and 5% thresholds.

In a preferred embodiment, MHC-binding agretopes are identified as the 9-mer frames that bind to several class II MHC alleles. In an especially preferred embodiment, MHC-binding agretopes are predicted to bind at least 10 alleles at 5% threshold and/or at least 5 alleles at 1% threshold. Such 9-mer frames may be especially likely to elicit an immune response in many members of the human population.

In a preferred embodiment, MHC-binding agretopes are predicted to bind MHC alleles that are present in at least 0.01-10% of the human population. Alternatively, to treat conditions that are linked to specific class II MHC alleles, MHC-binding agretopes are predicted to bind MHC alleles that are present in at least 0.01-10% of the relevant patient population.

Data about the prevalence of different MHC alleles in different ethnic and racial groups has been acquired by groups such as the National Marrow Donor Program (NMDP); for example see Mignot et al. Am. J. Hum. Genet. 68: 686-699 (2001), Southwood et al. J. Immunol. 160: 3363-3373 (1998), Hurley et al. Bone Marrow Transplantation 25: 136-137 (2000), Sintasath Hum. Immunol. 60: 1001 (1999), Collins et al. Tissue Antigens 55: 48 (2000), Tang et al. Hum. Immunol. 63: 221 (2002), Chen et al. Hum. Immunol. 63: 665 (2002), Tang et al. Hum. Immunol. 61: 820 (2000), Gans et al. Tissue Antigens 59: 364-369, and Baldassarre et al. Tissue Antigens 61: 249-252 (2003).

In a preferred embodiment, MHC binding agretopes are predicted for MHC heterodimers comprising highly prevalent MHC alleles. Class II MHC alleles that are present in at least 10% of the US population include but are not limited to: DPA1*0103, DPA1*0201, DPB1*0201, DPB1*0401, DPB1*0402, DQA1*0101, DQA1*0102, DQA1*0201, DQA1*0501, DQB1*0201, DQB1*0202, DQB1*0301, DQB1*0302, DQB1*0501, DQB1*0602, DRA*0101, DRB1*0701, DRB1*1501, DRB1*0301, DRB1*0101, DRB1*1101, DRB1*1301, DRB3*0101, DRB3*0202, DRB4*0101, DRB4*0103, and DRB5*0101.

In a preferred embodiment, MHC binding agretopes are also predicted for MHC heterodimers comprising moderately prevalent MHC alleles. Class II MHC alleles that are present in 1% to 10% of the US population include but are not limited to: DPA1*0104, DPA1*0302, DPA1*0301, DPB1*0101, DPB1*0202, DPB1*0301, DPB1*0501, DPB1*0601, DPB1*0901, DPB1*1001, DPB1*1101, DPB1*1301, DPB1*1401, DPB1*1501, DPB1*1701, DPB1*1901, DPB1*2001, DQA1*0103, DQA1*0104, DQA1*0301, DQA1*0302, DQA1*0401, DQB1*0303, DQB1*0402, DQB1*0502, DQB1*0503, DQB1*0601, DQB1*0603, DRB1*1302, DRB1*0404, DRB1*0801, DRB1*0102, DRB1*1401, DRB1*1104, DRB1*1201, DRB1*1503, DRB1*0901, DRB1*1601, DRB1*0407, DRB1*1001, DRB1*1303, DRB1*0103, DRB1*1502, DRB1*0302, DRB1*0405, DRB1*0402, DRB1*1102, DRB1*0803, DRB1*0408, DRB1*1602, DRB1*0403, DRB3*0301, DRB5*0102, and DRB5*0202.

MHC binding agretopes may also be predicted for MHC heterodimers comprising less prevalent alleles. Information about MHC alleles in humans and other species can be obtained, for example, from the IMGT/HLA sequence database (www.ebi.ac.uk/imgt/hla/).

In an additional preferred embodiment, MHC-binding agretopes are identified as the 9-mer frames that are located among “nested” agretopes, or overlapping 9-residue frames that are each predicted to bind a significant number of alleles. Such sequences may be especially likely to elicit an immune response.

Preferred MHC-binding agretopes are those agretopes that are predicted to bind, at a 3% threshold, to MHC alleles that are present in at least 5% of the population. Preferred MHC-binding agretopes in interferon beta include, but are not limited to, agretope 2: residues 5-13; agretope 3: residues 8-16; agretope 5: residues 15-23; agretope 6: residues 22-30; agretope 7: residues 30-38; agretope 8: residues 36-44; agretope 10: residues 57-65; agretope 11: residues 60-68; agretope 12: residues 63-71; agretope 13: residues 70-78; agretope 16: residues 122-130; agretope 18: residues 129-137; agretope 20: residues 143-151; agretope 21: residues 145-153; agretope 22: residues 146-154; agretope 23: residues 148-156; agretope 24: residues 151-159; agretope 25: residues 154-162; and agretope 26: residues 156-164.

Especially preferred MHC-binding agretopes are those agretopes that are predicted to bind, at a 1% threshold, to MHC alleles that are present in at least 10% of the population. Especially preferred MHC-binding agretopes in interferon beta include, but are not limited to, agretope 6: residues 22-30; agretope 8: residues 36-44; agretope 11: residues 60-68; agretope 20: residues 143-151; agretope 24: residues 151-159; and agretope 25: residues 154-162.

Additional especially preferred MHC-binding agretopes are those agretopes whose sequences partially overlap with additional MHC-binding agretopes. Sets of overlapping MHC-binding agretopes in interferon beta include, but are not limited to, residues 5-44; residues 57-78; residues 122--137; and residues 143-164.

Interferon beta is commonly used to treat multiple sclerosis. As multiple sclerosis has been linked to the presence of certain MHC alleles, especially preferred MHC-binding agretopes are those agretopes that are predicted to bind, at a 3% threshold, to MHC alleles that are commonly present in multiple sclerosis patients. The HLA DRB1*1501-DQB1*0602 haplotype has been repeatedly demonstrated to confer susceptibility to multiple sclerosis. Additional alleles that are associated with increased risk of multiple sclerosis include, but are not limited to, DRB1*1503 (Quelvennec et. al. Tissue Antigens 2003 61: 166-171), DPB1*0501 (Fukuzawa et. al. J. Neurol. 2000 247: 175-178), DRB1*1303 (Kohn et. al. Mult. Scler. 1999 5: 410-415), DQA1*0101 and DQA1*0102 (Arcos-Burgos et. al. Exp. Clin. Immunogenet. 1999 16: 131-138). Agretopes in interferon beta that are predicted to bind one or more MHC alleles which confer susceptibility to multiple sclerosis include, but are not limited to, agretope 10: residues 57-65; agretope 16 residues 122-130; and agretope 24: residues 151-159.

Confirmation of MHC-Binding Agretopes

In a preferred embodiment, the immunogenicity of the above-predicted MHC-binding agretopes is experimentally confirmed by measuring the extent to which peptides comprising each predicted agretope can elicit an immune response. However, it is possible to proceed from agretope prediction to agretope removal without the intermediate step of agretope confirmation.

Several methods, discussed in more detail below, can be used for experimental confirmation of agretopes. For example, sets of naive T cells and antigen presenting cells from matched donors can be stimulated with a peptide containing an agretope of interest, and T-cell activation can be monitored. It is also possible to first stimulate T cells with the whole protein of interest, and then re-stimulate with peptides derived from the whole protein. If sera are available from patients who have raised an immune response to interferon beta, it is possible to detect mature T cells that respond to specific epitopes. In a preferred embodiment, interferon gamma or IL-5 production by activated T-cells is monitored using Elispot assays, although it is also possible to use other indicators of T-cell activation or proliferation such as tritiated thymidine incorporation or production of other cytokines.

Patient Genotype Analysis and Screening

HLA genotype is a major determinant of susceptibility to specific autoimmune diseases (see for example Nepom Clin. Immunol. Immunopathol. 67: S50-S55 (1993)) and infections (see for example Singh et. al. Emerg. Infect. Dis. 3: 41-49 (1997)). Furthermore, the set of MHC alleles present in an individual can affect the efficacy of some vaccines (see for example Cailat-Zucman et. al. Kidney Int. 53: 1626-1630 (1998) and Poland et. al. Vaccine 20: 430-438 (2001)). HLA genotype may also confer susceptibility for an individual to elicit an unwanted immune response to a interferon beta therapeutic.

In a preferred embodiment, class II MHC alleles that are associated with increased or decreased susceptibility to elicit an immune response to interferon beta proteins are identified. For example, patients treated with interferon beta therapeutics may be tested for the presence of anti-interferon beta antibodies and genotyped for class II MHC. Alternatively, T-cell activation assays such as those described above may be conducted using cells derived from a number of genotyped donors. Alleles that confer susceptibility to interferon beta immunogenicity may be defined as those alleles that are significantly more common in those who elicit an immune response versus those who do not. Similarly, alleles that confer resistance to interferon beta immunogenicity may be defined as those that are significantly less common in those who do not elicit an immune response versus those that do. It is also possible to use purely computational techniques to identify which alleles are likely to recognize interferon beta therapeutics.

In one embodiment, the genotype association data is used to identify patients who are especially likely or especially unlikely to raise an immune response to a interferon beta therapeutic.

Design of active, less-immunogenic variants. In a preferred embodiment, the above-determined MHC-binding agretopes are replaced with alternate amino acid sequences to generate active variant interferon beta proteins with reduced or eliminated immunogenicity. Alternatively, the MHC-binding agretopes are modified to introduce one or more sites that are susceptible to cleavage during protein processing. If the agretope is cleaved before it binds to a MHC molecule, it will be unable to promote an immune response. There are several possible strategies for integrating methods for identifying less immunogenic sequences with methods for identifying structured and active sequences, including but not limited to those presented below.

In one embodiment, for one or more 9-mer agretope identified above, one or more possible alternate 9-mer sequences are analyzed for immunogenicity as well as structural and functional compatibility. The preferred alternate 9-mer sequences are then defined as those sequences that have low predicted immunogenicity and a high probability of being structured and active. It is possible to consider only the subset of 9-mer sequences that are most likely to comprise structured, active, less immunogenic variants. For example, it may be unnecessary to consider sequences that comprise highly non-conservative mutations or mutations that increase predicted immunogenicity.

In a preferred embodiment, less immunogenic variants of each agretope are predicted to bind MHC alleles in a smaller fraction of the population than the wild type agretope. In an especially preferred embodiment, the less immunogenic variant of each agretope is predicted to bind to MHC alleles that are present in not more than 5% of the population, with not more than 1% or 0.1% being most preferred.

Substitution matrices In another especially preferred embodiment, substitution matrices or other knowledge-based scoring methods are used to identify alternate sequences that are likely to retain the structure and function of the wild type protein. Such scoring methods can be used to quantify how conservative a given substitution or set of substitutions is. In most cases, conservative mutations do not significantly disrupt the structure and function of proteins (see for example, Bowie et. al. Science 247: 1306-1310 (1990), Bowie and Sauer Proc. Nat. Acad. Sci. USA 86: 2152-2156 (1989), and Reidhaar-Olson and Sauer Proteins 7: 306-316 (1990)). However, non-conservative mutations can destabilize protein structure and reduce activity (see for example, Lim et. al. Biochem. 31: 4324-4333 (1992)). Substitution matrices including but not limited to BLOSUM62 provide a quantitative measure of the compatibility between a sequence and a target structure, which can be used to predict non-disruptive substitution mutations (see Topham et al. Prot. Eng. 10: 7-21 (1997)). The use of substitution matrices to design peptides with improved properties has been disclosed; see Adenot et al. J. Mol. Graph. Model. 17: 292-309 (1999).Substitution matrices include, but are not limited to, the BLOSUM matrices (Henikoff and Henikoff, Proc. Nat. Acad. Sci. USA 89: 10917 (1992), the PAM matrices, the Dayhoff matrix, and the like. For a review of substitution matrices, see for example Henikoff Curr. Opin. Struct. Biol. 6: 353-360 (1996). It is also possible to construct a substitution matrix based on an alignment of a given protein of interest and its homologs; see for example Henikoff and Henikoff Comput. Appl. Biosci. 12: 135-143 (1996). In a preferred embodiment, each of the substitution mutations that are considered has a BLOSUM 62 score of zero or higher. According to this metric, preferred substitutions include, but are not limited to:

TABLE 1
Conservative mutations

| Wild type residue | Preferred substitutions |
|---|---|
| A | C S T A G V |
| C | C A |
| D | S N D E Q |
| E | S N D E Q H R K |
| F | M I L F Y W |
| G | S A G N |
| H | N E Q H R Y |
| I | M I L V F |
| K | S N E Q R K |
| L | M I L V F |
| M | Q M I L V F |
| N | S T G N D E Q H R K |
| P | P |
| Q | S N D E Q H R K M |
| R | N E Q H R K |
| S | S T A G N D E Q K |
| T | T A M I L V |
| V | S T A N V |
| W | F Y W |
| Y | H F Y W |

In addition, it is preferred that the total BLOSUM 62 score of an alternate sequence for a nine residue MHC-binding agretope is decreased only modestly when compared to the BLOSUM 62 score of the wild type nine residue agretope. In a preferred embodiment, the score of the variant 9mer is at least 50% of the wild type score, with at least 67%, 75% or 90% being especially preferred.

Alternatively, alternate sequences can be selected that minimize the absolute reduction in BLOSUM score; for example it is preferred that the score decrease for each 9-mer is less than 20, with score decreases of less than about 10 or about 5 being especially preferred. The exact value may be chosen to produce a library of alternate sequences that is experimentally tractable and also sufficiently diverse to encompass a number of active, stable, less immunogenic variants.

In a preferred embodiment, substitution mutations are preferentially introduced at positions that are substantially solvent exposed. As is known in the art, solvent exposed positions are typically more tolerant of mutation than positions that are located in the core of the protein.

In a preferred embodiment, substitution mutations are preferentially introduced at positions that are not highly conserved. As is known in the art, positions that are highly conserved among members of a protein family are often important for protein function, stability, or structure, while positions that are not highly conserved often may be modified without significantly impacting the structural or functional properties of the protein.

Alanine substitutions. In an alternate embodiment, one or more alanine substitutions may be made, regardless of whether an alanine substitution is conservative or non-conservative. As is known in the art, incorporation of sufficient alanine substitutions may be used to disrupt intermolecular interactions.

In a preferred embodiment, variant 9-mers are selected such that residues that have been or can be identified as especially critical for maintaining the structure or function of interferon beta retain their wild type identity. In alternate embodiments, it may be desirable to produce variant interferon beta proteins that do not retain wild type activity. In such cases, residues that have been identified as critical for function may be specifically targeted for modification.

Mutagenesis studies have identified three residues, R27, R35, and K123, that are important for interferon beta activity [Runkel et. al. J. Biol. Chem. 1998 273: 8003-8008]. A number of additional residues in interferon beta have been implicated in receptor binding, based on the results of cassette alanine scanning experiments, including F15, Q16, Q18, K19, W22, Q23, L28, E29, Y30, L32, K33, M36, N37, D39, E42, Q64, F67, A68, R71, Q72, D73, Y92, N96, K99, T100, R128, L130, H131, K134, V148, R152, Y155, and N158 [Runkel et. al. Biochem 2000 39: 2538-2551]. However, since these residues were mutated in groups, it is likely that only a subset are actually required for interferon beta function.

Protein design methods and MHC agretope identification methods may be used together to identify stable, active, and minimally immunogenic protein sequences (see WO03/006154). The combination of approaches provides significant advantages over the prior art for immunogenicity reduction, as most of the reduced immunogenicity sequences identified using other techniques fail to retain sufficient activity and stability to serve as therapeutics.

Protein design methods may identify non-conservative or unexpected mutations that nonetheless confer desired functional properties and reduced immunogenicity, as well as identifying conservative mutations. Nonconservative mutations are defined herein to be all substitutions not included in Table 1 above; nonconservative mutations also include mutations that are unexpected in a given structural context, such as mutations to hydrophobic residues at the protein surface and mutations to polar residues in the protein core.

Furthermore, protein design methods may identify compensatory mutations. For example, if a given first mutation that is introduced to reduce immunogenicity also decreases stability or activity, protein design methods may be used to find one or more additional mutations that serve to recover stability and activity while retaining reduced immunogenicity. Similarly, protein design methods may identify sets of two or more mutations that together confer reduced immunogenicity and retained activity and stability, even in cases where one or more of the mutations, in isolation, fails to confer desired properties.

In a preferred embodiment, the results of matrix method calculations are used to identify which of the 9 amino acid positions within the agretope(s) contribute most to the overall binding propensities for each particular allele “hit”. This analysis considers which positions (P1-P9) are occupied by amino acids which consistently make a significant contribution to MHC binding affinity for the alleles scoring above the threshold values. Matrix method calculations are then used to identify amino acid substitutions at said positions that would decrease or eliminate predicted immunogenicity and PDA® technology is used to determine which of the alternate sequences with reduced or eliminated immunogenicity are compatible with maintaining the structure and function of the protein.

In an alternate preferred embodiment, the residues in each agretope are first analyzed by one skilled in the art to identify alternate residues that are potentially compatible with maintaining the structure and function of the protein. Then, the set of resulting sequences are computationally screened to identify the least immunogenic variants. Finally, each of the less immunogenic sequences are analyzed more thoroughly in PDA® technology protein design calculations to identify protein sequences that maintain the protein structure and function and decrease immunogenicity.

In an alternate preferred embodiment, each residue that contributes significantly to the MHC binding affinity of an agretope is analyzed to identify a subset of amino acid substitutions that are potentially compatible with maintaining the structure and function of the protein. This step may be performed in several ways, including PDA® calculations or visual inspection by one skilled in the art. Sequences may be generated that contain all possible combinations of amino acids that were selected for consideration at each position. Matrix method calculations can be used to determine the immunogenicity of each sequence. The results can be analyzed to identify sequences that have significantly decreased immunogenicity. Additional PDA® calculations may be performed to determine which of the minimally immunogenic sequences are compatible with maintaining the structure and function of the protein.

In an alternate preferred embodiment, pseudo-energy terms derived from the peptide binding propensity matrices are incorporated directly into the PDA® technology calculations. In this way, it is possible to select sequences that are active and less immunogenic in a single computational step.

Combining immunogenicity reduction strategies In a preferred embodiment, more than one method is used to generate variant proteins with desired functional and immunological properties. For example, substitution matrices may be used in combination with PDA® technology calculations. Strategies for immunogenicity reduction include, but are not limited to, those described in U.S. Ser. No. _______, Optimized Fc Variants and Methods for Generation, filed Mar. 26, 2004, incorporated by reference.

In a preferred embodiment, a variant protein with reduced binding affinity for one or more class II MHC alleles is further engineered to confer improved solubility. As protein aggregation may contribute to unwanted immune responses, increasing protein solubility may reduce immunogenicity (see for example SIFN).

In an additional preferred embodiment, a variant protein with reduced binding affinity for one or more class II MHC alleles is further modified by derivitization with PEG or another molecule. As is known in the art, PEG may sterically interfere with antibody binding or improve protein solubility, thereby reducing immunogenicity. In an especially preferred embodiment, rational PEGylation methods are used (see U.S. Ser. No. 60/459,094 and U.S. Ser. No. _______, “Generating Protein ProDrugs using Reversible PPG Linkages, filed Mar. 19, 2004, hereby incorporated by reference).

In a preferred embodiment, PDA® technology and matrix method calculations are used to remove more than one MHC-binding agretope from a protein of interest.

Additional modifications. Additional insertions, deletions, and substitutions may be incorporated into the variant interferon proteins of the invention in order to confer other desired properties.

In a preferred embodiment, the immunogenicity of interferons may be modulated. See for example U.S. Ser. Nos.: 09/903,378; 10/039,170; 10/339,788 (filed Jan. 8, 2003, titled Novel Protein with Altered Immunogenicity); and PCT/US01/21823; and PCT/US02/00165. All references expressly incorporated by reference in their entirety. See for example U.S. Ser. Nos.: 09/903,378; 10/039,170; __/______ (filed Jan. 8, 2003, titled Novel Protein with Altered Immunogenicity); and PCT/US01/21823; and PCT/US02/00165. All references cited herein are expressly incorporated by reference in their entirety.

In an alternate preferred embodiment, the interferon variant is further modified to increase stability. As discussed above, modifications that improve stability can also improve solubility, for example by decreasing the concentration of partially unfolded, aggregation-prone species. For example, modifications can be introduced to the protein core that improve packing or remove polar or charged groups that are not forming favorable hydrogen bond or electrostatic interactions. It is also possible to introduce modifications that introduce stabilizing electrostatic interactions or remove destabilizing interactions. Additional stabilizing modifications also may be used.

In one embodiment, the sequence of the variant interferon protein is modified in order to add or remove one or more N-linked or O-linked glycosylation sites. Addition of glycosylation sites to variant interferon polypeptides may be accomplished, for example, by the incorporation of one or more serine or threonine residues to the native sequence or variant interferon polypeptide (for O-linked glycosylation sites) or by the incorporation of a canonical N-linked glycosylation site, including but not limited to, N-X-Y, where X is any amino acid except for proline and Y is preferably threonine, serine or cysteine. Glycosylation sites may be removed by replacing one or more serine or threonine residues or by replacing one or more canonical N-linked glycosylation sites.

In another preferred embodiment, one or more cysteine, lysine, histidine, or other reactive amino acids are designed into variant interferon proteins in order to incorporate labeling sites or PEGylation sites. It is also possible to remove one or more cysteine, lysine, histidine, or other reactive amino acids in order to prevent the incorporation of labeling sites or PEGylations sites at specific locations. For example, in a preferred embodiment, non-labile PEGylation sites are selected to be well removed from any required receptor binding sites in order to minimize loss of activity.

Variant interferon polypeptides of the present invention may also be modified to form chimeric molecules comprising a variant interferon polypeptide fused to another, heterologous polypeptide or amino acid sequence. In one embodiment, such a chimeric molecule comprises a fusion of a variant interferon polypeptide with a tag polypeptide which provides an epitope to which an anti-tag antibody can selectively bind. The epitope tag is generally placed at the amino-or carboxyl-terminus of the variant interferon polypeptide. The presence of such epitope-tagged forms of a variant interferon polypeptide can be detected using an antibody against the tag polypeptide. Also, provision of the epitope tag enables the variant interferon polypeptide to be readily purified by affinity purification using an anti-tag antibody or another type of affinity matrix that binds to the epitope tag. Various tag polypeptides and their respective antibodies are well known in the art. Examples include poly-histidine (poly-His) or poly-histidine-glycine (poly-His-Gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto [Evan et al., Molecular an Cellular Biology, 5:3610-3616 (1985)]; and the Herpes Simplex virus glycoprotein D (gD) tag and its antibody [Paborsky et al., Protein Engineering, 3(6): 547-553 (1990)]. Other tag polypeptides include the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. NatI. Acad. Sci. U.S.A. 87:6393-6397 (1990)].

In an alternative embodiment, the chimeric molecule may comprise a fusion of a variant interferon polypeptide with another protein. Various fusion partners are well known in the art, and include but are not limited to the following examples. The variant interferon proteins of the invention may be fused to an immunoglobulin or the Fc region of an immunoglobulin, such as an IgG molecule. The interferon variants can also be fused to albumin, other interferon proteins, other cytokine proteins, the extracellular domains of the interferon receptor protein, etc.immunoglobulin or a particular region of an immunoglobulin. For a bivalent form of the chimeric molecule, such a fusion could be to the Fc region of an IgG molecule.

In another embodiment, the N- and C-termini of a variant IFN protein are joined to create a cyclized or circularly permutated IFN protein. Various techniques may be used to permutate proteins. See U.S. Pat. No. 5,981,200; Maki K, Iwakura M., Seikagaku. 2001 January; 73(1): 42-6; Pan T., Methods Enzymol. 2000; 317:313-30; Heinemann U, Hahn M., Prog Biophys Mol Biol. 1995; 64(2-3): 121-43; Harris M E, Pace N R, Mol Biol Rep. 1995-96; 22(2-3):115-23; Pan T, Uhlenbeck OC., Mar.30, 1993; 125(2): 111-4; Nardulli A M, Shapiro D J. 1993 Winter; 3(4):247-55, EP 1098257 A2; WO 02/22149; WO 01/51629; WO 99/51632; Hennecke, et al., 1999, J. Mol. Biol., 286, 1197-1215; Goldenberg et al J. Mol. Biol 165, 407-413 (1983); Luger et al, Science, 243, 206-210 (1989); and Zhang et al., Protein Sci 5, 1290-1300 (1996); all hereby incorporated by reference.

To produce a circularly permuted IFN protein, a novel set of N- and C-termini are created at amino acid positions normally internal to the protein's primary structure, and the original N- and C-termini are joined via a peptide linker consisting of from 0 to 30 amino acids in length (in some cases, some of the amino acids located near the original termini are removed to accommodate the linker design). In a preferred embodiment, the novel N- and C-termini are located in a non-regular secondary structural element, such as a loop or turn, such that the stability and activity of the novel protein are similar to those of the original protein. The circularly permuted IFN protein may be further PEGylated, glycosylated, or otherwise modified. In a further preferred embodiment PDA® technology may be used to further optimize the IFN variant, particularly in the regions affected by circular permutation. These include the novel N- and C-termini, as well as the original termini and linker peptide.

In addition, a completely cyclic IFNTPO may be generated, wherein the protein contains no termini. This is accomplished utilizing intein technology. Thus, peptides can be cyclized and in particular inteins may be utilized to accomplish the cyclization.

Generating the variants. Variant interferon nucleic acids and proteins of the invention may be produced using a number of methods known in the art and described herein.

Preparing nucleic acids encoding the IFN variants. In a preferred embodiment, nucleic acids encoding IFN variants are prepared by total gene synthesis, or by site-directed mutagenesis of a nucleic acid encoding wild type or variant IFN protein. Methods including template-directed ligation, recursive PCR, cassette mutagenesis, site-directed mutagenesis or other techniques that are well known in the art may be utilized (see for example Strizhov et. al. PNAS 93:15012-15017 (1996), Prodromou and Perl, Prot. Eng. 5: 827-829 (1992), Jayaraman and Puccini, Biotechniques 12: 392-398 (1992), and Chalmers et. at. Biotechniques 30: 249-252 (2001)).

Expression vectors. In a preferred embodiment, an expression vector that comprises the components described below and a gene encoding a variant IFN protein is prepared. Numerous types of appropriate expression vectors and suitable regulatory sequences for a variety of host cells are known in the art for a variety of host cells. The expression vectors may contain transcriptional and translational regulatory sequences including but not limited to promoter sequences, ribosomal binding sites, transcriptional start and stop sequences, translational start and stop sequences, transcription terminator signals, polyadenylation signals, and enhancer or activator sequences. In a preferred embodiment, the regulatory sequences include a promoter and transcriptional start and stop sequences. In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences, which flank the expression construct. The integrating vector may be directed to a specific locus in the host cell by selecting the appropriate homologous sequence for inclusion in the vector. Constructs for integrating vectors are well known in the art. In addition, in a preferred embodiment, the expression vector contains a selectable marker gene to allow the selection of transformed host cells. Selection genes are well known in the art and will vary with the host cell used. The expression vectors may be either self-replicating extrachromosomal vectors or vectors which integrate into a host genome.

The expression vector may include a secretory leader sequence or signal peptide sequence that provides for secretion of the variant IFN protein from the host cell. Suitable secretory leader sequences that lead to the secretion of a protein are known in the art. The signal sequence typically encodes a signal peptide comprised of hydrophobic amino acids, which direct the secretion of the protein from the cell, as is well known in the art. The protein is either secreted into the growth media or, for prokaryotes, into the periplasmic space, located between the inner and outer membrane of the cell. For expression in bacteria, usually bacterial secretory leader sequences, operably linked to a variant IFN encoding nucleic acid, are usually preferred.

Transfection/Transformation. The variant IFN nucleic acids are introduced into the cells either alone or in combination with an expression vector in a manner suitable for subsequent expression of the nucleic acid. The method of introduction is largely dictated by the targeted cell type, as discussed below. Exemplary methods include CaPO4 precipitation, liposome fusion, Llipofectin®, electroporation, viral infection, dextran-mediated transfection, polybrene mediated transfection, protoplast fusion, direct microinjection, etc. The variant IFN nucleic acids may stably integrate into the genome of the host cell or may exist either transiently or stably in the cytoplasm. As outlined herein, a particularly preferred method utilizes retroviral infection, as outlined in PCT/US97/01019, incorporated by reference.

Appropriate host cells for the expression of IFN variants. Appropriate host cells for the expression of IFN variants include yeast, bacteria, archaebacteria, fungi, and insect and animal cells, including mammalian cells. Of particular interest are bacteria such as E. coli and Bacillus subtilis, fungi such as Saccharomyces cerevisiae, Pichia pastoris, and Neurospora, insects such as Drosophila melangaster and insect cell lines such as SF9, mammalian cell lines including 293, CHO, COS, Jurkat, NIH3T3, etc (see the ATCC cell line catalog, hereby expressly incorporated by reference), as well as primary cell lines.

Interferon variants can also be produced in more complex organisms, including but not limited to plants (such as corn, tobacco, and algae) and animals (such as chickens, goats, cows); see for example Dove, Nature Biotechnol. 20: 777-779 (2002).

In one embodiment, the cells may be additionally genetically engineered, that is, contain exogenous nucleic acid other than the expression vector comprising the variant IFN nucleic acid.

Expression methods. The variant IFN proteins of the present invention are produced by culturing a host cell transformed with an expression vector containing nucleic acid encoding a variant IFN protein, under the appropriate conditions to induce or cause expression of the variant IFN protein. The conditions appropriate for variant IFN protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Purification. In a preferred embodiment, the IFN variants are purified or isolated after expression. Standard purification methods include electrophoretic, molecular, immunological and chromatographic techniques, including ion exchange, hydrophobic, affinity, and reverse-phase HPLC chromatography, and chromatofocusing. For example, a IFN variant may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, NY, 3dr ed. (1994). The degree of purification necessary will vary depending on the desired use, and in some instances no purification will be necessary. For further references on purification of type I interferons, see for example Moschera et. al. Meth. Enzym. 119: 177-192 (1986).For further references on purification of type I interferons, see for example Moschera et. al. Meth. Enzym. 119: 177-183 (1986); Tarnowski et. al. Meth. Enzym. 119:153-165(1986); Thatcher et. al. Meth. Enzym. 119:166-177 (1986); Lin et. al. Meth. Enzym. 119:183-192 (1986). Methods for purification of interferon beta are disclosed in U.S. Pat. Nos. 4,462,940 and 4,894,330.

Posttranslational modification and derivitization. Once made, the variant IFN proteins may be covalently modified. Covalent and non-covalent modifications of the protein are thus included within the scope of the present invention. Such modifications may be introduced into a variant IFN polypeptide by reacting targeted amino acid residues of the polypeptide with an organic derivatizing agent that is capable of reacting with selected side chains or terminal residues. Optimal sites for modification can be chosen using a variety of criteria, including but not limited to, visual inspection, structural analysis, sequence analysis and molecular simulation.

In one embodiment, the variant IFN proteins of the invention are labeled with at least one element, isotope or chemical compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position. Labels include but are not limited to biotin, tag (e.g. FLAG, Myc) and fluorescent labels (e.g. fluorescein).

Derivatization with bifunctional agents is useful, for instance, for cross linking a variant IFN protein to a water-insoluble support matrix or surface for use in the method for purifying anti-variant IFN antibodies or screening assays, as is more fully described below. Commonly used cross linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio] propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “-amino groups of lysine, arginine, and histidine side chains ([T. E. Creighton, Proteins: Structure and Molecular Properties, W. H. Freeman & Co., San Francisco, pp. 79-86 (1983))], acetylation of the N-terminal amine, and amidation of any C-terminal carboxyl group.

Such derivitization may improve the solubility, absorption, permeability across the blood brain barrier, serum half life, and the like. Modifications of variant IFN polypeptides may alternatively eliminate or attenuate any possible undesirable side effect of the protein. Moieties capable of mediating such effects are disclosed, for example, in Remington's Pharmaceutical Sciences, 16th ed., Mack Publishing Co., Easton, Pa. (1980).

Another type of covalent modification of variant IFN comprises linking the variant IFN polypeptide to one of a variety of nonproteinaceous polymers, e.g., polyethylene glycol (“PEG”), polypropylene glycol, or polyoxyalkylenes, in the manner set forth in U.S. Pat. Nos. 4,640,835; 4,496,689; 4,301,144; 4,670,417; 4,791,192 or 4,179,337. A variety of coupling chemistries may be used to achieve PEG attachment, as is well known in the art. Examples, include but are not limited to, the technologies of Shearwater and Enzon, which allow modification at primary amines, including but not limited to, cysteine groups, histidine groups, lysine groups and the N- terminus. S (see, Kinstler et al, Advanced Drug Deliveries Reviews, 54, 477-485 (2002) and M J Roberts et al, Advanced Drug Delivery Reviews, 54, 459-476 (2002), both hereby incorporated by reference). Both labile and non-labile PEG linkages may be used.

Addition of Nobex polymers and the like. An additional form of covalent modification includes coupling of the variant IFN polypeptide with one or more molecules of a polymer comprised of a lipophililic and a hydrophilic moiety. Such composition may enhance resistance to hydrolytic or enzymatic degradation of the IFN protein. Polymers utilized may incorporate, for example, fatty acids for the lipophilic moiety and linear polyalkylene glycols for the hydrophilic moiety. The polymers may additionally incorporate acceptable sugar moieties as well as spacers used for IFN protein attachment. Polymer compositions and methods for covalent conjugation are described, for example, in U.S. Pat. Nos. 5,681,811; 5,359,030.

Another type of modification is chemical or enzymatic coupling of glycosides to the variant IFN protein. Such methods are described in the art, e.g., in WO 87/05330 published 11 Sep. 1987, and in Aplin and Wriston, CRC Crit. Rev. Biochem., pp. 259-306 (1981).

Alternatively, removal of carbohydrate moieties present on the variant IFN polypeptide may be accomplished chemically or enzymatically. Chemical deglycosylation techniques are known in the art and described, for instance, by Hakimuddin, et al., Arch. Biochem. Biophys., 259:52 (1987) and by Edge et al., Anal. Biochem., 118: 131 (1981). Enzymatic cleavage of carbohydrate moieties on polypeptides can be achieved by the use of a variety of endo-and exo-glycosidases as described by Thotakura et al., Meth. Enzymol., 138:350 (1987).

A primary object of the invention is the identification of variant interferon proteins with improved solubility. Accordingly, in a preferred embodiment, the variant interferon proteins are assayed for solubility using methods including but not limited to those described below.

In all preferred embodiments, the variant and wild-type proteins are usually compared directly in the same assay system and under the same conditions in order to evaluate the solubility of each variant.

The solubility of the interferon variant proteins may be determined under a number of solution conditions. A variety of excipients, including solubilizing and stabilizing agents, may be tested for their ability to promote the highest stable IFN concentration. In addition, different salt concentrations and varying pH may be tested. In a preferred embodiment, solubility is assayed under pharmaceutically acceptable conditions.

In order to evaluate the effectiveness of the modifications introduced to the protein, suitable biophysical measurements must be taken. In this instance, the protein concentration over time, relative mobility on native gel electrophoresis, and oligomeric state are the primary criteria of interest. In the case of poor solubility, aggregates will form over time in the protein solution, and eventually precipitate entirely. In the latter case, a drop in solution protein concentration over time will be observed, following centrifugation and sampling of the solution phase. A description of techniques to evaluate oligomeric state, including detection of aggregates, follows below (DLS, AUC, SEC, etc).

To determine the impact of mutations introduced to the molecule, variant and wild-type proteins should be compared directly in the same assay system. When comparable data are available, the evaluation of the influence of a given mutation (the variant) on solubility may be rendered.

Soluble expression. The variables to be tested in order to achieve expression of IFN as a soluble protein rather than in inclusion bodies are to lower the fermentation temperature, to lower the concentration of the inducing agent, and to attempt the application of a commercially-available enzyme system engineered to to optimize soluble protein expression (Athena Expression Systems, Baltimore, Md.).

Maximum concentration under different solution conditions. A variety of excipients, including solubilizing and stabilizing agents will be tested for their ability to promote the highest stable IFNb concentration. In addition, different salt concentrations and varying pH will be tested. The tests utilized will be the same as in the assay of protein solubility, because essentially the solubility of the protein at varying high concentrations will be tested in this methodology.

Besides native gel electrophoresis, these are the methods of choice for evaluation of protein oligomeric state.

Dynamic light scattering (DLS)—DLS is a method useful for determination of diffusion coefficients based on signal correlation from fluctuation of laser light scattered from Brownian motion of particles in solution (Light scattering by Polymer Solutions, Paul C. Heimenz, Chapter 10 in Polymer Chemistry, Marcel Dekker, Inc., NY, 1984, pp. 659-701). Commercially- available instruments provide graphical or table readouts of particle population(s) by size(s) after transforming the diffusion coefficient(s) measured by deconvolution/autocorrelation of laser light scattering data using the Stokes-Einstein equation. The size is therefore the hydrodynamic radius. Particle size standards aremay be used to check the accuracy of the instrument settings (nanoparticles obtained from Duke Scientific Corporation, Palo Alto, Calif.). The distribution of particle sizes within a population(s) is the dispersity, and this factor provides data on the uniformity of the particle population(s). Both dispersity and the appearance of aggregates over time may be monitored to test for solubility—this should be monitored as well as the appearance of aggregates over time for an indication of stability.

Aggregated protein will may be easily resolved by DLS, and readily detected at low levels due to the physical property of aggregates: they scatter more laser light per unit due to the greater target surface area. Concentration requirements can be on the order of mg/ml to obtain reasonable signal, however as little as 10 microliter in some model instruments can be detected, for a total sample amount requirement on the order of 10 microgram per sample condition tested (allowing additional material for sample handling losses). The sample is may be directly introduced into the cuvette (i.e. it is not necessary to perform a chromatographic step first) and not separated on a separate matrix requiring time and removal from the test solution conditions (in the event of a differently formulated mobile phase) as in the case of static light scattering. A relative ratio of monodisperse to aggregate particle population is generally available from this line of instruments, sometimes weighted by mass sometimes by light-scattering intensity, and sometimes a choice of either, depending on software provided by the manufacturermay be determined. Optionally, this ratio may be weighted by mass or by light scattering intensity. Thus, DLS is an idea preferred technique to monitor formation of aggregates, and holds the advantage in that it is a non-intrusive technique.

Analytical ultracentrifugation (AUC). In another preferred embodiment (AUC) is used to determine the oligomerization state of the variant interferon proteins. AUC can be performed in two different ‘modes’, either velocity or equilibrium. Equilibrium AUC can be considered the ‘gold standard’ for determining protein molecular weight and oligomeric state measurement.

Size exclusion chromatography (SEC)—A further preferred embodiment is to use size-exclusion chromatography (SEC) to determine the oligomerization state of the variant interferon proteins. Utilizing high performance liquid chromatography, sample may be introduced to an isocratic mobile phase and separated on a gel permeation matrix designed to exclude protein on the basis of size. Thus, the samples will be “sieved” such that the aggregated protein will elute first with the shortest retention time, and will be easily separated from the remainder. This will unequivocallycan identify aggregates and allow a relative quantification by peak integration using the peak analysis software provided with the instrument.

In an alternate embodiment, protein concentration is monitored as a function of time. In the case of poor solubility, aggregates will form over time in the protein solution, and eventually precipitate entirely. This may be performed following centrifugation and sampling of the solution phase, in which case insolubility can be measured as a drop in solution protein concentration over time will be observed following centrifugation.

In an alternate embodiment, the oligomerization state is determined by monitoring relative mobility on native gel electrophoresis.

In another embodiment, the amount of protein that is expressed solubly in a prokaryotic host is determined. While factors other than the solubility of the native protein can impact levels of soluble expression, improvements in soluble expression may correlate with improvements in solubility. Any of a number of methods may be used; for example, following expression, SDS-polyacrylamide gel electrophoresis and/or western blots can be done on the soluble fraction of crude cell lysates or the expression media. There are also high throughput screens for soluble expression. In one embodiment, the protein of interest is fused to a fluorescent protein such as GFP, and the cells monitored for fluorescence (Waldo et. al. Nat. Biotechnol. 17: 691 (1999)). In an alternate embodiment, the protein of interest-is fused to the antibiotic resistance enzyme chloramphenicol transferase. If the protein expresses solubly, the enzyme will be functional, thereby allowing growth on media with increased concentration of the antibiotic chloramphenicol (Maxwell et. al. Protein Sci. 8: 1908 (1999)). In another embodiment, the protein of interest is expressed as a fusion with the alpha domain of the enzyme beta-galactosidase. If the protein expresses in soluble form, the alpha domain will complement the omega domain to yield a functional enzyme. This may be detected as blue rather than white colony formation when the cells are plated on media containing the indicator X-gal (Wigley et. al. Nat. Biotechnol. 19: 131 (2001)).

Assaying the activity of the variants. In a preferred embodiment, the wild-type and variant proteins are analyzed for biological activities by suitable methods known in the art. Such assays include but are not limited to activation of interferon-responsive genes, receptor binding assays, antiviral activity assays, cytopathic effect inhibition assays, (Familletti et. al., Meth. Enzymol. 78:387-394), antiproliferative assays, (Aebersold and Sample, Meth. Enzymol. 119:579-582), immunomodulatory assays (U.S. Pat. Nos. 4,914,033; 4,753,795), and assays that monitor the induction of MHC molecules (for example, Hokland et al, Meth. Enzymol. 119:688-693)., as described in Meager, J. Immunol. Meth., 261:21-36 (2002).

In a preferred embodiment, wild type and variant proteins will be analyzed for their ability to activate interferon-sensitive signal transduction pathways. One example is the interferon-stimulated response element (ISRE) assay, described below and in the Examples. Cells which constitutively express the type I interferon receptor (for example Hela cells, 293T cells) are transiently transfected with an ISRE-luciferase vector. After transfection, the cells are treated with an interferon variant. In a preferred embodiment, a number of protein concentrations, for example from 0.0001-10 ng/mL, are tested to generate a dose-response curve. In an alternate embodiment, two or more concentrations are tested. If the variant binds and activates its receptor, the resulting signal transduction cascade induces luciferase expression. Luminseescence can be measured in a number of ways, for example by using a TopCount™ or Fusion™ microplate reader.

In a preferred embodiment, wild type and variant proteins will be analyzed for their ability to bind to the type I interferon receptor (IFNAR). Suitable binding assays include, but are not limited to, BlAcore assays (Pearce et al., Biochemistry 38:81-89 (1999)) and AlphaScreen™ assays (commercially available from PerkinElmer) (Bosse R., IIIy C., and Chelsky D (2002). Principles of AlphaScreen™ PerkinElmer Literature Application Note Ref# s4069. AlphaScreen™ is a bead-based non-radioactive luminescent proximity assay where the donor beads are excited by a laser at 680 nm to release singlet oxygen. The singlet oxygen diffuses and reacts with the thioxene derivative on the surface of acceptor beads leading to fluorescence emission at ˜600 nm. The fluorescence emission occurs only when the donor and acceptor beads are brought into close proximity by molecular interactions occurring when each is linked to ligand and receptor respectively. This ligand-receptor interaction can be competed away using receptor-binding variants while non-binding variants will not compete.

In an alternate preferred embodiment, wild type and variant proteins will be analyzed for their efficacy in treating an animal model of disease, such as the mouse or rat EAE model for multiple sclerosis.

In an alternate preferred embodiment, wild type and variant proteins will be analyzed for their antiviral activity.

Antiproliferative activity: In an alternate preferred embodiment, wild type and variant proteins will be analyzed for their efficacy in treating an animal model of disease, such as the mouse or rat EAE model for multiple sclerosis.

Determining the immunogenicity of the variants In the case of engineered MHC immunogenicity, the variants are tested. Also, in some cases, increased protein solubility may decrease immunogenicity by reducing uptake by antigen presenting cells. Accordingly, in a preferred embodiment, uptake of wild type and variant interferon proteins by professional antigen presenting cells is monitored.

In a preferred embodiment, the immunogenicity of the interferon variants is determined experimentally to confirm that the variants do have reduced or eliminated immunogenicity relative to the parent protein. Again the discussion below is centered around IFN-β, but the methods can be utilized for any IFN of the invention.

In a preferred embodiment, ex vivo T-cell activation assays are used to experimentally quantitate immunogenicity. In this method, antigen presenting cells and naive T cells from matched donors are challenged with a peptide or whole protein of interest one or more times. Then, T cell activation can be detected using a number of methods, for example by monitoring production of cytokines or measuring uptake of tritiated thymidine. In the most preferred embodiment, interferon gamma production is monitored using Elispot assays (see Schmittel et. al. J. Immunol. Meth., 24: 17-24 (2000)).

Other suitable T-cell assays include those disclosed in Meidenbauer, et al. Prostate 43, 88-100 (2000); Schultes, B. C and Whiteside, T. L., J. Immunol. Methods 279, 1-15 (2003); and Stickler, et al., J. Immunotherapy, 23, 654-660 (2000).

In a preferred embodiment, the PBMC donors used for the above-described T-cell activation assays will comprise class II MHC alleles that are common in patients requiring treatment for interferon beta responsive disorders. For example, for most diseases and disorders, it is desirable to test donors comprising all of the alleles that are prevalent in the population. However, for diseases or disorders that are linked with specific MHC alleles, it may be more appropriate to focus screening on alleles that confer susceptibility to interferon beta responsive disorders.

In a preferred embodiment, the MHC haplotype of PBMC donors or patients that raise an immune response to the wild type or variant interferon beta are compared with the MHC haplotype of patients who do not raise a response. This data may be used to guide preclinical and clinical studies as well as aiding in identification of patients who will be especially likely to respond favorably or unfavorably to the interferon beta therapeutic.

In an alternate preferred embodiment, immunogenicity is measured in transgenic mouse systems. For example, mice expressing fully or partially human class II MHC molecules may be used.

In an alternate embodiment, immunogenicity is tested by administering the interferon beta variants to one or more animals, including rodents and primates, and monitoring for antibody formation. Non-human primates with defined MHC haplotypes may be especially useful, as the sequences and hence peptide binding specificities of the MHC molecules in non-human primates may be very similar to the sequences and peptide binding specificities of humans. Similarly, genetically engineered mouse models expressing human MHC peptide-binding domains may be used (see for example Sonderstrup et. al. Immunol. Rev. 172: 335-343 (1999) and Forsthuber et. al. J. Immunol. 167: 119-125 (2001)).

Administration and Treatment using IFN variants. Once made, the variant IFN proteins and nucleic acids of the invention find use in a number of applications. In a preferred embodiment, a variant IFN protein or nucleic acid is administered to a patient to treat an IFN related disorder.

The administration of the variant IFN proteins of the present invention, preferably in the form of a sterile aqueous solution, may be done in a variety of ways, including, but not limited to, orally, parenterally, subcutaneously, intravenously, intranasally, transdermally, intraperitoneally, intramuscularly, intrapulmonary, vaginally, rectally, intranasally or intraocularly. In some instances, the variant IFN protein may be directly applied as a solution or spray. Depending upon the manner of introduction, the pharmaceutical composition may be formulated in a variety of ways.

The pharmaceutical compositions of the present invention comprise a variant IFN protein in a form suitable for administration to a patient. In the preferred embodiment, the pharmaceutical compositions are in a water-soluble form, such as being present as pharmaceutically acceptable salts, which is meant to include both acid and base addition salts.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations, and discussed above.

In a further embodiment, the variant IFN proteins are added in a micellular formulation; see U.S. Pat. No. 5,833,948, hereby expressly incorporated by reference in its entirety.

Combinations of pharmaceutical compositions may be administered. Moreover, the compositions may be administered in combination with other therapeutics.

In a preferred embodiment, the nucleic acid encoding the variant IFN proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. The oligonucleotides canmay be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection [(Dzau et al., Trends in Biotechnology 11:205-210 (1993))]. In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990). For review of gene marking and gene therapy protocols see Anderson et al., Science 256:808-813 (1992).

While the foregoing invention has been described above, it will be clear to one skilled in the art that various changes and additional embodiments made be made without departing from the scope of the invention. All publications, patents, patent applications (provisional, utility and PCT) or other documents cited herein are incorporated by references in their entirety.

### EXAMPLES

### Example 1

### Construction of a Homology Model of Interferon Kappa

A homology model of interferon kappa was constructed based on the sequence of human interferon kappa (GenBank code 14488028), the crystal structures for interferon tau (PDB code 1BL5) and interferon beta (PDB code 1AU1), as well as the NMR structure for interferon alpha-2a (PDB code 1ITF). The sequences for interferons alpha-2a, beta, kappa, and tau were aligned using the multiple sequence alignment tool in the Homology model of the InsightII software package (Accelrys), as shown in FIG. 2. As the sequences share only approximately 35% identity, slightly different sequence alignments could have been used instead (see for example LaFleur et. al. J. Biol. Chem. 276: 39765-39771 (2001)). Based on similarity to the other interferon sequences, disulfide bonds are expected to be formed between residues C3 and C102 and between residues C32 and C155 (LaFleur supra); these disufides were used as constraints in the generation of the homology models. A total of nine homology models were generated using the Modeler tool in the InsightII software package (Accelrys). The structures were analyzed for quality and the top four models were used in the analysis and design calculations described below.

### Example 2

### Identification of Exposed Hydrophobic Residues in Type I Interferons

A number of type I interferon structures were analyzed to identify solvent-exposed hydrophobic residues. The absolute and fractional solvent-exposed hydrophobic surface area of each residue was calculated using the method of Lee and Richards (J. Mol. Biol. 55: 379-400 (1971)) using an add-on radius of 1.4 Å (Angstroms). Each residue was also classified as core, boundary, or surface (see Dahiyat and Mayo Science 278: 82-87 (1997)).

Solvent exposed hydrophobic residues in interferon-alpha 2a were defined to be hydrophobic residues with at least 75 Å² (square Angstroms) exposed hydrophobic surface area in the interferon alpha-2a NMR structure (PDB code 1ITF, first molecule).

TABLE 1
Exposed hydrophobic residues in interferon-alpha 2a.

| | | core/ | exposed | |
|---|---|---|---|---|
| | | boundary/ | hydrophobic | percent hydrophobic |
| residue | # | surface | surface area | area exposed |
| MET | 16 | surface | 93.90 | 44.50 |
| PHE | 27 | surface | 172.10 | 69.10 |
| LEU | 30 | surface | 84.20 | 39.40 |
| TYR | 89 | surface | 80.00 | 41.10 |
| ILE | 100 | surface | 103.60 | 50.00 |
| LEU | 110 | surface | 151.30 | 70.20 |
| MET | 111 | surface | 76.40 | 35.60 |
| LEU | 117 | surface | 78.60 | 37.80 |
| LEU | 128 | surface | 104.30 | 50.40 |
| LEU | 161 | surface | 90.10 | 45.30 |

Solvent exposed hydrophobic residues in interferon beta were defined to be hydrophobic residues with at least 75 Å² (square Angstroms) exposed hydrophobic surface area in the interferon-beta crystal structure (PDB code 1AU1, chain A)

TABLE 2
Exposed hydrophobic residues in interferon-beta.

| | | | exposed | |
|---|---|---|---|---|
| | | core/surface/ | hydrophobic | percent hydrophobic |
| residue | # | boundary | surface area | area buried |
| LEU | 5 | boundary | 100.30 | 48.30 |
| PHE | 8 | surface | 131.00 | 54.90 |
| PHE | 15 | surface | 151.90 | 63.30 |
| TRP | 22 | surface | 147.90 | 58.30 |
| LEU | 28 | boundary | 61.90 | 31.00 |
| TYR | 30 | surface | 129.00 | 66.80 |
| LEU | 32 | surface | 50.40 | 23.70 |
| MET | 36 | boundary | 82.60 | 40.00 |
| LEU | 47 | boundary | 72.20 | 35.50 |
| TYR | 92 | surface | 84.60 | 44.40 |
| PHE | 111 | surface | 196.30 | 80.10 |
| LEU | 116 | surface | 94.60 | 45.70 |
| LEU | 120 | surface | 67.20 | 32.50 |
| LEU | 130 | surface | 57.10 | 27.40 |
| VAL | 148 | boundary | 77.40 | 42.80 |
| TYR | 155 | surface | 88.60 | 46.30 |

Solvent exposed hydrophobic residues in interferon-kappa were defined to be hydrophobic residues with at least 30 Å² (square Angstroms) exposed hydrophobic surface area in at least one of the top four homology models (see above) and which were classified as boundary (B) or surface (S) in at least 3 of the 4 top structures. Solvent exposed hydrophobic residues in interferon kappa, along with their exposed hydrophobic surface area and C/S/B classification, are shown below.

TABLE 3
Exposed hydrophobic residues in interferon kappa. Solvent exposed
hydrophobic surface areas in square Angstroms are given for the
top four homology models. Core/surface/boundary classification
is indicated as “C”, “S”, or “B” below.

| | | model 1 | | model 2 | | model 3 | | model 4 | |
|---|---|---|---|---|---|---|---|---|---|
| LEU | 1 | 134.57 | S | 135.88 | B | 91.03 | B | 134.11 | S |
| LEU | 5 | 102.62 | S | 89.78 | B | 70.67 | S | 103.39 | S |
| VAL | 8 | 70.36 | S | 76.97 | S | 70.19 | S | 72.51 | S |
| TRP | 15 | 155.63 | S | 161.08 | S | 149.83 | S | 153.22 | S |
| LEU | 18 | 33.86 | B | 42.72 | B | 64.82 | B | 34.39 | B |
| PHE | 28 | 39.03 | S | 32.47 | B | 16.19 | B | 34.43 | S |
| VAL | 30 | 118.49 | S | 112.38 | S | 43.12 | S | 118.23 | S |
| LEU | 33 | 92.00 | S | 73.35 | S | 72.73 | S | 93.60 | S |
| ILE | 37 | 106.52 | B | 127.16 | B | 99.30 | B | 106.28 | B |
| LEU | 46 | 84.43 | S | 86.04 | S | 84.47 | S | 83.90 | S |
| TYR | 48 | 79.98 | B | 60.73 | B | 93.88 | B | 81.91 | B |
| MET | 52 | 101.62 | B | 149.86 | S | 149.37 | S | 104.68 | S |
| LEU | 65 | 109.14 | B | 98.21 | S | 111.58 | B | 91.38 | S |
| PHE | 68 | 55.88 | B | 107.51 | B | 104.30 | B | 57.45 | B |
| PHE | 76 | 61.69 | B | 66.90 | B | 53.90 | B | 59.28 | B |
| TYR | 78 | 104.70 | B | 112.65 | S | 135.51 | B | 111.51 | B |
| TRP | 79 | 57.96 | S | 138.78 | B | 133.03 | C | 58.32 | S |
| ILE | 88 | 104.67 | S | 77.94 | S | 77.75 | S | 111.79 | S |
| TYR | 96 | 98.61 | B | 118.35 | B | 63.52 | B | 97.46 | B |
| MET | 111 | 118.98 | B | 152.74 | S | 115.40 | B | 109.32 | B |
| MET | 114 | 141.73 | S | 188.48 | S | 174.59 | S | 134.99 | B |
| MET | 119 | 147.52 | S | 173.09 | S | 159.56 | S | 134.72 | S |
| VAL | 126 | 23.49 | C | 77.29 | S | 70.45 | B | 54.01 | S |
| LEU | 132 | 86.27 | S | 95.70 | S | 81.83 | S | 84.16 | S |
| TYR | 150 | 41.55 | B | 62.57 | B | 86.01 | B | 45.22 | B |
| VAL | 160 | 49.02 | B | 69.23 | S | 70.61 | B | 49.02 | B |
| TYR | 167 | 99.52 | S | 84.23 | S | 149.46 | S | 100.52 | S |
| TYR | 170 | 63.85 | S | 77.37 | S | 110.88 | S | 61.83 | S |

The results in Table 3 were combined with the sequence analysis described in Example 4 to identify exposed hydrophobic residues in interferon kappa that could be replaced with polar residues without compromising the structure or function of the resulting variant protein.

Solvent exposed hydrophobic residues in ovine interferon tau were defined to be hydrophobic residues that were at least 25% exposed to solvent in the crystal structure of interferon tau (PDB code 1B5L).

TABLE 4
Exposed hydrophobic residues in interferon-tau.
The exposed hydrophobic surface areas

| | | | | Percent |
|---|---|---|---|---|
| | | C/S/B | Exposed | hydrophobic area |
| Residue | # | classification | hydrophobic area | burial |
| TYR | 2 | surface | 153.9 | 22.9 |
| LEU | 9 | surface | 85.8 | 59.1 |
| LEU | 24 | boundary | 121.1 | 42.5 |
| LEU | 30 | surface | 152.2 | 25.8 |
| TYR | 69 | surface | 71.6 | 62.5 |
| TRP | 77 | surface | 233.3 | 6.3 |
| MET | 114 | surface | 137.6 | 36.9 |
| VAL | 118 | surface | 103.9 | 42.9 |
| TYR | 136 | boundary | 53.3 | 72.6 |
| VAL | 146 | boundary | 64.5 | 63.9 |

### Example 3

### Identification of Dimer Interface Residues in Type I Interferons

Potential sites of interactions between interferon monomers were identified by examining contacts between monomers in the crystal structures of interferon molecules.

Interferon alpha-2b crystallized as a trimer of dimers (PDB code 1RH2), in which the dimer interface is zinc-mediated (see Radhakrishnan et. al. Structure 4: 1453-1463 (1996)). The zinc-mediated dimer is referred to herein as the “AB dimer”, while the interface between AB dimers is referred to as the “BC” dimer interface. The zinc-binding site comprises the residues Glu 41 and Glu 42. Additional residues that have been implicated in stabilizing the AB dimer interface include Lys 121, Asp 114, Gly 44, and Arg 33 (Radhakrishnan, supra).

Next, distance measurements were used to identify additional residues that may participate in intermolecular interactions. Residues that are within 8 Å (Angstroms) of the AB dimer interface (as measured by CA-CA distances) include: 35-37, 39-41, 44-46, 114-115, 117-118, 121-122, and 125. Residues that are within 8 Å of the BC dimer-dimer interface (as measured by CA-CA distances) include: 16, 19, 20, 25, 27, 28, 30, 33, 54, 58, 61, 65, 68, 85, 93, 99, 112, 113, and 149.

Interferon beta crystallized as an asymmetric dimer (PDB code 1AU1). Residues that are within 5 Å of the dimer interface (minimum heavy atom-heavy atom distance) include 42, 43, 46-49, 51, 113, 116, 117, 120, 121, and 124 (on chain A), as well as 1-6, 8, 9, 12, 16, 93, 96, 97, 100, 101, and 104 (on chain B).

### Example 4

### Identification of Residues Observed at Each Position in the Interferon Family

A large number of type I interferon sequences are known to exist, comprising interferons of different subtypes (e.g. alpha-2, alpha4, beta, kappa), allelic variants (e.g. alpha-2a vs. alpha-2b), and interferons from different species. Analysis of these different interferon sequences can suggest substitutions that will be compatible with maintaining the structure and function of type I interferons.

The BLAST sequence alignment program was used to identify the 100 protein sequences in the nonredundant protein sequence database that are most closely related to interferon kappa. The annotations for these sequences were analyzed to confirm that all of the sequences are type one interferons. Next, the number of occurrences of each residue (and of deletions, denoted “−”) at each position in interferon kappa was determined. For example, the frequency of each residue at the exposed hydrophobic positions in interferon kappa is shown below.

TABLE 5
Frequency of each residue at exposed hydrophobic positions in interferon kappa.

| # | wt | — | A | C | D | E | F | G | H | I | K | L | M | N | P | Q | R | S | T | V | W | Y |
|---|---|---|---|---|---|---|---|---|---|---|---|---|---|---|---|---|---|---|---|---|---|---|
| 1 | L | 0 | 0 | 0 | 0 | 0 | 0 | 0 | 0 | 0 | 0 | 39 | 0 | 0 | 0 | 0 | 0 | 0 | 0 | 0 | 0 | 0 |
| 5 | L | 0 | 0 | 0 | 0 | 0 | 0 | 0 | 0 | 0 | 0 | 39 | 0 | 0 | 0 | 0 | 0 | 0 | 0 | 0 | 0 | 0 |
| 8 | V | 0 | 0 | 0 | 5 | 0 | 0 | 0 | 1 | 1 | 0 | 0 | 0 | 15 | 0 | 0 | 0 | 2 | 12 | 3 | 0 | 0 |
| 15 | W | 17 | 0 | 0 | 0 | 0 | 0 | 0 | 0 | 0 | 0 | 5 | 2 | 0 | 0 | 0 | 8 | 1 | 2 | 0 | 4 | 0 |
| 18 | L | 0 | 0 | 0 | 0 | 0 | 1 | 0 | 0 | 0 | 0 | 73 | 0 | 0 | 0 | 0 | 0 | 0 | 0 | 0 | 0 | 0 |
| 28 | F | 10 | 0 | 0 | 0 | 0 | 3 | 0 | 0 | 0 | 0 | 0 | 0 | 0 | 1 | 0 | 0 | 70 | 0 | 0 | 0 | 0 |
| 30 | V | 0 | 1 | 0 | 0 | 0 | 16 | 0 | 44 | 0 | 0 | 0 | 0 | 0 | 2 | 0 | 4 | 9 | 0 | 8 | 0 | 0 |
| 33 | L | 0 | 0 | 0 | 0 | 0 | 0 | 0 | 0 | 0 | 0 | 88 | 0 | 0 | 0 | 2 | 0 | 0 | 0 | 0 | 0 | 0 |
| 37 | I | 0 | 0 | 0 | 0 | 0 | 0 | 0 | 4 | 3 | 51 | 0 | 5 | 12 | 1 | 0 | 12 | 0 | 1 | 0 | 0 | 1 |
| 46 | L | 0 | 1 | 0 | 0 | 0 | 12 | 0 | 0 | 0 | 6 | 13 | 0 | 0 | 0 | 0 | 0 | 0 | 0 | 58 | 0 | 0 |
| 48 | Y | 0 | 0 | 0 | 1 | 0 | 0 | 78 | 0 | 0 | 0 | 5 | 0 | 0 | 1 | 1 | 0 | 0 | 0 | 1 | 0 | 3 |
| 52 | M | 1 | 0 | 0 | 0 | 0 | 0 | 0 | 0 | 0 | 0 | 0 | 3 | 0 | 2 | 80 | 4 | 0 | 0 | 0 | 0 | 0 |
| 65 | L | 0 | 0 | 0 | 0 | 0 | 0 | 0 | 0 | 0 | 0 | 3 | 0 | 0 | 0 | 87 | 0 | 0 | 0 | 0 | 0 | 0 |
| 68 | F | 0 | 0 | 0 | 0 | 0 | 85 | 0 | 0 | 0 | 0 | 2 | 0 | 0 | 0 | 0 | 0 | 1 | 0 | 1 | 0 | 1 |
| 76 | F | 0 | 11 | 1 | 0 | 0 | 4 | 0 | 0 | 0 | 0 | 0 | 0 | 0 | 1 | 0 | 0 | 73 | 0 | 0 | 0 | 0 |
| 78 | Y | 0 | 68 | 0 | 0 | 0 | 0 | 0 | 0 | 0 | 0 | 0 | 1 | 0 | 0 | 0 | 0 | 5 | 9 | 4 | 0 | 3 |
| 79 | W | 0 | 0 | 0 | 0 | 0 | 0 | 0 | 0 | 0 | 0 | 0 | 0 | 0 | 0 | 0 | 0 | 0 | 0 | 0 | 90 | 0 |
| 89 | I | 0 | 12 | 0 | 2 | 0 | 0 | 0 | 0 | 4 | 0 | 0 | 0 | 1 | 0 | 0 | 0 | 3 | 68 | 0 | 0 | 0 |
| 97 | Y | 0 | 0 | 0 | 46 | 6 | 0 | 5 | 26 | 0 | 0 | 0 | 0 | 4 | 0 | 0 | 0 | 0 | 0 | 0 | 0 | 3 |
| 112 | M | 43 | 13 | 0 | 0 | 0 | 6 | 0 | 0 | 1 | 0 | 1 | 5 | 0 | 3 | 0 | 0 | 1 | 10 | 7 | 0 | 0 |
| 115 | M | 19 | 4 | 0 | 0 | 3 | 0 | 6 | 0 | 5 | 1 | 8 | 16 | 0 | 0 | 0 | 0 | 27 | 1 | 0 | 0 | 0 |
| 120 | M | 38 | 0 | 0 | 0 | 0 | 0 | 0 | 0 | 1 | 0 | 5 | 44 | 0 | 0 | 0 | 0 | 0 | 0 | 2 | 0 | 0 |
| 127 | V | 77 | 0 | 0 | 0 | 0 | 0 | 4 | 0 | 0 | 0 | 2 | 3 | 0 | 0 | 0 | 0 | 0 | 0 | 4 | 0 | 0 |
| 133 | L | 4 | 0 | 0 | 0 | 0 | 0 | 0 | 0 | 0 | 0 | 64 | 0 | 0 | 0 | 0 | 0 | 0 | 0 | 22 | 0 | 0 |
| 151 | Y | 0 | 0 | 0 | 1 | 0 | 0 | 0 | 13 | 0 | 0 | 0 | 0 | 0 | 0 | 0 | 0 | 0 | 0 | 0 | 0 | 76 |
| 161 | V | 0 | 21 | 0 | 0 | 0 | 0 | 0 | 0 | 0 | 0 | 3 | 17 | 0 | 0 | 0 | 0 | 0 | 0 | 49 | 0 | 0 |
| 168 | Y | | | | | | | | | | | | | | | | | 12 | | | | |
| 171 | Y | 0 | 0 | 0 | 0 | 0 | 0 | 0 | 0 | 0 | 0 | 0 | 0 | 6 | 0 | 0 | 0 | 0 | 12 | 0 | 0 | 3 |

The raw frequencies above were normalized using the method of Henikoff & Henikoff (J. Mol. Biol. 243: 547-578 (1994)). Numerical values are only included for cells in which the number of occurrences in the table above is greater than 0.

TABLE 6
Normalized frequency of each residue at exposed hydrophobic positions in interferon kappa.

| # | wt | — | A | C | D | E | F | G | H | I | K | L | M | N | P | Q | R | S | T | V | W | Y |
|---|---|---|---|---|---|---|---|---|---|---|---|---|---|---|---|---|---|---|---|---|---|---|
| 1 | L | — | — | — | — | — | — | — | — | — | — | 0.6 | — | — | — | — | — | — | — | — | — | — |
| 5 | L | — | — | — | — | — | — | — | — | — | — | 0.6 | — | — | — | — | — | — | — | — | — | — |
| 8 | V | — | — | — | 0 | — | — | — | 0 | 0 | — | — | — | 0.1 | — | — | — | 0.1 | 0.2 | 0.2 | — | — |
| 15 | W | — | — | — | — | — | — | — | — | — | — | 0.1 | 0.1 | — | — | — | 0 | 0 | 0.1 | — | 0.2 | — |
| 18 | L | — | — | — | — | — | 0 | — | — | — | — | 0.7 | — | — | — | — | — | — | — | — | — | — |
| 28 | F | — | — | — | — | — | 0.2 | — | — | — | — | — | — | — | 0 | — | — | 0.4 | — | — | — | — |
| 30 | V | — | 0 | — | — | — | 0.2 | — | 0.1 | — | — | — | — | — | 0 | — | 0 | 0.3 | — | 0.2 | — | — |
| 33 | L | — | — | — | — | — | — | — | — | — | — | 1 | — | — | — | 0 | — | — | — | — | — | — |
| 37 | I | — | — | — | — | — | — | — | 0.1 | 0.2 | 0.1 | — | 0.1 | 0.3 | 0 | — | 0.1 | — | 0 | — | — | 0 |
| 46 | L | — | 0 | — | — | — | 0.2 | — | — | — | 0.2 | 0.5 | — | — | — | — | — | — | — | 0.2 | — | — |
| 48 | Y | — | — | — | 0 | — | — | 0.6 | — | — | — | 0.1 | — | — | 0 | 0 | — | — | — | 0 | — | 0.2 |
| 52 | M | — | — | — | — | — | — | — | — | — | — | — | 0.2 | — | 0 | 0.8 | 0 | — | — | — | — | — |
| 65 | L | — | — | — | — | — | — | — | — | — | — | 0.2 | — | — | — | 0.8 | — | — | — | — | — | — |
| 68 | F | — | — | — | — | — | 0.9 | — | — | — | — | 0 | — | — | — | — | — | 0 | — | 0 | — | 0 |
| 76 | F | — | 0.2 | 0 | — | — | 0.2 | — | — | — | — | — | — | — | 0 | — | — | 0.5 | — | — | — | — |
| 78 | Y | — | 0.4 | — | — | — | — | — | — | — | — | — | 0 | — | — | — | — | 0 | 0.3 | 0.1 | — | 0.2 |
| 79 | W | — | — | — | — | — | — | — | — | — | — | — | — | — | — | — | — | — | — | — | 1 | — |
| 89 | I | — | 0.4 | — | 0.1 | — | — | — | — | 0.2 | — | — | — | 0 | — | — | — | 0 | 0.4 | — | — | — |
| 97 | Y | — | — | — | 0.4 | 0 | — | 0 | 0.3 | — | — | — | — | 0.1 | — | — | — | — | — | — | — | 0.2 |
| 112 | M | — | 0.2 | — | — | — | 0.2 | — | — | 0 | — | 0 | 0.2 | — | 0 | — | — | 0 | 0.1 | 0 | — | — |
| 115 | M | — | 0 | — | — | 0 | — | 0.2 | — | 0.1 | 0 | 0.2 | 0.3 | — | — | — | — | 0.1 | 0 | — | — | — |
| 120 | M | — | — | — | — | — | — | — | — | 0 | — | 0 | 0.3 | — | — | — | — | — | — | 0 | — | — |
| 127 | V | — | — | — | — | — | — | 0 | — | — | — | 0.1 | 0 | — | — | — | — | — | — | 0.2 | — | — |
| 133 | L | — | — | — | — | — | — | — | — | — | — | 0.9 | — | — | — | — | — | — | — | 0 | — | — |
| 151 | Y | — | — | — | 0 | — | — | — | 0.3 | — | — | — | — | — | — | — | — | — | — | — | — | 0.7 |
| 161 | V | — | 0.4 | — | — | — | — | — | — | — | — | 0 | 0.1 | — | — | — | — | — | — | 0.5 | — | — |
| 168 | Y | | | | | | | | | | | | | | | | | 0.4 | | | | |
| 171 | Y | — | — | — | — | — | — | — | — | — | — | — | — | 0.2 | — | — | — | — | 0.4 | — | — | 0.2 |

This sequence alignment data was used in conjunction with the PDA® technology calculations described above to identify suitable residues for different variable positions. If hydrophobicity at a given position was found to be conserved among interferons (i.e. the frequency of polar residues at that position was zero or very low), the position was not considered further. At the remaining positions, PDA® technology calculations were performed to aid in the identification of suitable polar replacements.

Exposed hydrophobic positions at which polar residues are observed with a normalized frequency of 0.1 or greater include:

TABLE 7
Exposed hydrophobic positions in interferon-kappa at which
polar residues are observed with a normalized frequency
of at least 0.1 in other interferon proteins.

| # | wt | — | A | D | E | G | H | K | N | Q | R | S | T |
|---|---|---|---|---|---|---|---|---|---|---|---|---|---|
| 8 | V | — | — | 0 | — | — | 0 | — | 0.1 | — | — | 0.1 | 0.2 |
| 15 | W | — | — | — | — | — | — | — | — | — | 0 | 0 | 0.1 |
| 28 | F | — | — | — | — | — | — | — | — | — | — | 0.4 | — |
| 30 | V | — | 0 | — | — | — | 0.1 | — | — | — | 0 | 0.3 | — |
| 37 | I | — | — | — | — | — | 0.1 | 0.1 | 0.3 | — | 0.1 | — | 0 |
| 46 | L | — | 0 | — | — | — | — | 0.2 | — | — | — | — | — |
| 48 | Y | — | — | 0 | — | 0.6 | — | — | — | 0 | — | — | — |
| 52 | M | — | — | — | — | — | — | — | — | 0.8 | 0 | — | — |
| 65 | L | — | — | — | — | — | — | — | — | 0.8 | — | — | — |
| 76 | F | — | 0.2 | — | — | — | — | — | — | — | — | 0.5 | — |
| 78 | Y | — | 0.4 | — | — | — | — | — | — | — | — | 0 | 0.3 |
| 89 | I | — | 0.4 | 0.1 | — | — | — | — | 0 | — | — | 0 | 0.4 |
| 97 | Y | — | — | 0.4 | 0 | 0 | 0.3 | — | 0.1 | — | — | — | — |
| 112 | M | — | 0.2 | — | — | — | — | — | — | — | — | 0 | 0.1 |
| 115 | M | — | 0 | — | 0 | 0.2 | — | 0 | — | — | — | 0.1 | 0 |
| 151 | Y | — | — | 0 | — | — | 0.3 | — | — | — | — | — | — |
| 161 | V | — | 0.4 | — | — | — | — | — | — | — | — | — | — |
| 168 | Y | — | — | — | — | — | — | — | — | — | — | 0.4 | — |
| 171 | Y | — | — | — | — | — | — | — | 0.2 | — | — | — | 0.4 |

### Example 5

### Identification of Suitable Replacements for Exposed Hydrophobic Residues

PDA® technology calculations were performed to identify polar residues that are compatible with the structure and function of type I interferons. Energies were calculated for alanine and each of the polar residues at each exposed hydrophobic position, using a force field describing van der Waals interactions (VDW), electrostatics (Elec), hydrogen bonds (Hbond), and solvation (Solv). The energy of the wild type hydrophobic residue was also calculated. Polar residues with total energy that were similar to or more favorable than the wild type hydrophobic residue (the first line below for each position) were considered to be compatible with the target interferon (* below), and the polar residues with the most favorable energies were especially preferred (** below). Histidine was modeled in two possible states: “HSP” is the doubly-protonated state of histidine, while “HIS” is neutral histidine.

TABLE 8
Interferon-alpha calculation results, exposed hydrophobic residues

| | # | AA | Total | VDW | Elec | HBond | Solv |
|---|---|---|---|---|---|---|---|
| | 16 | MET | 9.68 | −4.05 | 0.00 | 0.00 | 13.729 |
| * | 16 | ALA | 3.87 | −1.65 | 0.00 | 0.00 | 5.522 |
| ** | 16 | ASP | −1.33 | −2.85 | −0.40 | 0.00 | 1.9233 |
| * | 16 | GLU | 1.55 | −3.19 | −0.40 | 0.00 | 5.1371 |
| * | 16 | HIS | 3.90 | −3.60 | 0.00 | 0.00 | 7.4983 |
| * | 16 | HSP | 3.91 | −3.62 | 0.27 | 0.00 | 7.2511 |
| * | 16 | LYS | 5.22 | −3.31 | 0.31 | 0.00 | 8.2164 |
| * | 16 | ASN | 0.86 | −2.88 | 0.01 | 0.00 | 3.7346 |
| * | 16 | GLN | 0.70 | −3.20 | −0.04 | 0.00 | 3.9397 |
| * | 16 | ARG | 0.73 | −3.36 | 0.22 | 0.00 | 3.8702 |
| * | 16 | SER | 0.00 | −1.94 | 0.00 | 0.00 | 1.9394 |
| * | 16 | THR | 3.55 | −2.89 | 0.04 | 0.00 | 6.4007 |
| | 27 | PHE | 20.55 | −2.52 | 0.00 | 0.00 | 23.0764 |
| * | 27 | ALA | 6.99 | −0.82 | 0.00 | 0.00 | 7.8098 |
| * | 27 | ASP | 1.27 | −1.51 | −0.38 | 0.00 | 3.1569 |
| * | 27 | GLU | 1.76 | −1.53 | −0.22 | 0.00 | 3.5092 |
| * | 27 | HIS | 11.57 | −1.76 | −0.01 | 0.00 | 13.3424 |
| * | 27 | HSP | 11.16 | −1.76 | 0.16 | 0.00 | 12.7635 |
| * | 27 | LYS | 7.36 | −2.10 | 0.25 | 0.00 | 9.2138 |
| ** | 27 | ASN | 0.52 | −1.52 | −0.06 | 0.00 | 2.091 |
| ** | 27 | GLN | 0.89 | −1.54 | 0.00 | 0.00 | 2.4286 |
| * | 27 | ARG | 5.35 | −1.59 | 0.21 | 0.00 | 6.7299 |
| * | 27 | SER | 1.63 | −1.00 | −0.03 | 0.00 | 2.6514 |
| * | 27 | THR | 6.62 | −1.40 | −0.03 | 0.00 | 8.0523 |
| | 100 | ILE | 6.17 | −4.09 | 0.00 | 0.00 | 10.2668 |
| * | 100 | ALA | 3.44 | −1.47 | 0.00 | 0.00 | 4.9013 |
| * | 100 | ASP | −0.59 | −2.28 | 0.24 | 0.00 | 1.4537 |
| ** | 100 | GLU | −1.26 | −3.19 | 0.50 | 0.00 | 1.4374 |
| | 100 | HIS | 15.87 | 0.86 | −0.01 | 0.00 | 15.0219 |
| | 100 | HSP | 15.16 | 0.98 | −0.20 | 0.00 | 14.3823 |
| * | 100 | LYS | 1.23 | −3.37 | −0.38 | 0.00 | 4.9902 |
| * | 100 | ASN | 0.38 | −3.14 | 0.00 | 0.00 | 3.5252 |
| ** | 100 | GLN | −2.56 | −3.28 | 0.02 | 0.00 | 0.7041 |
| ** | 100 | ARG | −1.57 | −3.39 | −0.27 | 0.00 | 2.0909 |
| * | 100 | SER | −0.30 | −1.72 | −0.01 | 0.00 | 1.4346 |
| * | 100 | THR | 4.32 | −2.62 | 0.00 | 0.00 | 6.9432 |
| | 110 | LEU | 18.52 | −1.89 | 0.00 | 0.00 | 20.4107 |
| * | 110 | ALA | 8.94 | −0.77 | 0.00 | 0.00 | 9.7089 |
| * | 110 | ASP | 3.92 | −1.36 | 0.17 | 0.00 | 5.1126 |
| * | 110 | GLU | 4.44 | −2.34 | 0.61 | 0.00 | 6.1639 |
| * | 110 | HIS | 13.80 | −1.79 | 0.00 | 0.00 | 15.5913 |
| * | 110 | HSP | 13.11 | −1.79 | −0.10 | 0.00 | 15.0058 |
| * | 110 | LYS | 11.14 | −1.96 | −0.23 | 0.00 | 13.3274 |
| ** | 110 | ASN | 2.75 | −1.37 | −0.04 | 0.00 | 4.1649 |
| ** | 110 | GLN | 2.83 | −2.34 | 0.06 | 0.00 | 5.1235 |
| * | 110 | ARG | 6.17 | −0.09 | −0.23 | 0.00 | 6.4996 |
| ** | 110 | SER | 3.03 | −0.94 | −0.02 | 0.00 | 3.9872 |
| * | 110 | THR | 4.82 | −1.84 | −0.03 | 0.00 | 6.7023 |
| | 111 | MET | 1.37 | −4.94 | 0.00 | 0.00 | 6.308 |
| | 111 | ALA | 5.58 | −1.21 | 0.00 | 0.00 | 6.7846 |
| * | 111 | ASP | 0.88 | −2.06 | 0.41 | 0.00 | 2.534 |
| * | 111 | GLU | 0.33 | −2.52 | 0.42 | 0.00 | 2.4273 |
| | 111 | HIS | 2.55 | −3.90 | −0.01 | 0.00 | 6.4709 |
| | 111 | HSP | 3.57 | −3.92 | −1.10 | 0.00 | 8.5877 |
| | 111 | LYS | 2.18 | −2.62 | −0.28 | 0.00 | 5.0789 |
| * | 111 | ASN | 0.14 | −2.09 | 0.05 | 0.00 | 2.1808 |
| ** | 111 | GLN | −0.92 | −2.54 | −0.05 | 0.00 | 1.6617 |
| * | 111 | ARG | 1.21 | −2.71 | −0.44 | 0.00 | 4.3527 |
| * | 111 | SER | 1.29 | −1.46 | 0.02 | 0.00 | 2.7337 |
| ** | 111 | THR | −0.16 | −3.15 | 0.05 | 0.00 | 2.9415 |
| | 117 | LEU | 3.03 | −4.07 | 0.00 | 0.00 | 7.0989 |
| * | 117 | ALA | −1.03 | −1.74 | 0.00 | 0.00 | 0.7126 |
| ** | 117 | ASP | −3.58 | −3.54 | 0.63 | 0.00 | −0.6613 |
| ** | 117 | GLU | −3.35 | −3.35 | 0.26 | 0.00 | −0.2511 |
| | 117 | HIS | 3.54 | −3.46 | −0.08 | 0.00 | 7.0827 |
| | 117 | HSP | 3.69 | −3.26 | 0.46 | 0.00 | 6.5019 |
| * | 117 | LYS | −1.42 | −4.06 | −0.48 | 0.00 | 3.1122 |
| * | 117 | ASN | −0.83 | −3.24 | −0.11 | 0.00 | 2.5211 |
| ** | 117 | GLN | −4.34 | −3.37 | 0.06 | 0.00 | −1.0372 |
| ** | 117 | ARG | −3.91 | −1.54 | −0.49 | −2.87 | 0.9774 |
| ** | 117 | SER | −3.47 | −2.09 | −0.03 | 0.00 | −1.3545 |
| * | 117 | THR | −1.87 | −3.00 | −0.02 | 0.00 | 1.1538 |
| | 161 | LEU | 10.25 | −3.57 | 0.00 | 0.00 | 13.8222 |
| * | 161 | ALA | 2.72 | −1.25 | 0.00 | 0.00 | 3.9705 |
| * | 161 | ASP | −0.17 | −2.59 | −0.04 | −0.11 | 2.5728 |
| ** | 161 | GLU | −2.33 | −3.04 | 0.15 | 0.00 | 0.5566 |
| * | 161 | HIS | 2.94 | −4.91 | −0.03 | 0.00 | 7.8882 |
| * | 161 | HSP | 4.64 | −4.93 | −0.19 | 0.00 | 9.7575 |
| ** | 161 | LYS | −1.13 | −3.55 | −0.20 | 0.00 | 2.6196 |
| * | 161 | ASN | −0.29 | −2.17 | −0.07 | 0.00 | 1.943 |
| * | 161 | GLN | −0.66 | −3.07 | −0.03 | 0.00 | 2.4459 |
| * | 161 | ARG | −0.43 | −4.56 | −1.02 | −4.78 | 9.9354 |
| * | 161 | SER | 0.34 | −1.58 | −0.04 | 0.00 | 1.9577 |
| * | 161 | THR | 0.71 | −2.75 | −0.04 | 0.00 | 3.4958 |

TABLE 9
Interferon beta calculation results, exposed hydrophobic residues

| | # | AA | Total | VDW | Elec | HBond | Solv |
|---|---|---|---|---|---|---|---|
| | 5 | LEU | 6.86 | −4.43 | 0.00 | 0.00 | 11.28 |
| * | 5 | ALA | 1.42 | −1.74 | 0.00 | 0.00 | 3.16 |
| ** | 5 | ASP | −2.63 | −2.74 | −0.37 | 0.00 | 0.47 |
| ** | 5 | GLU | −3.43 | −3.98 | −0.31 | 0.00 | 0.87 |
| | 5 | HIS | 13.88 | −0.11 | −0.09 | 0.00 | 14.07 |
| | 5 | HSP | 13.62 | −0.01 | 0.08 | 0.00 | 13.55 |
| * | 5 | LYS | −0.35 | −4.39 | 0.18 | 0.00 | 3.86 |
| * | 5 | ASN | −0.15 | −2.77 | 0.02 | 0.00 | 2.61 |
| ** | 5 | GLN | −3.95 | −4.00 | −0.03 | 0.00 | 0.08 |
| * | 5 | ARG | 0.17 | −3.17 | 0.21 | 0.00 | 3.12 |
| ** | 5 | SER | −3.45 | −2.03 | −0.02 | 0.00 | −1.40 |
| ** | 5 | THR | −2.86 | −3.43 | −0.02 | 0.00 | 0.59 |
| | 8 | PHE | 11.34 | −4.41 | 0.00 | 0.00 | 15.75 |
| * | 8 | ALA | −0.23 | −1.77 | 0.00 | 0.00 | 1.54 |
| ** | 8 | ASP | −3.43 | −2.73 | −0.34 | 0.00 | −0.37 |
| ** | 8 | GLU | −2.58 | −4.05 | −0.30 | 0.00 | 1.77 |
| * | 8 | HIS | 6.12 | −3.53 | 0.08 | 0.00 | 9.57 |
| * | 8 | HSP | 6.14 | −3.54 | 0.47 | 0.00 | 9.20 |
| * | 8 | LYS | 2.74 | −3.94 | 0.24 | 0.00 | 6.44 |
| * | 8 | ASN | −1.13 | −2.74 | −0.02 | 0.00 | 1.63 |
| ** | 8 | GLN | −2.86 | −2.46 | −0.08 | −2.76 | 2.44 |
| * | 8 | ARG | −1.50 | −4.00 | 0.33 | 0.00 | 2.17 |
| ** | 8 | SER | −4.37 | −2.02 | −0.02 | 0.00 | −2.33 |
| * | 8 | THR | 3.32 | −3.02 | −0.08 | 0.00 | 6.42 |
| | 15 | PHE | 16.43 | −3.32 | 0.00 | 0.00 | 19.75 |
| * | 15 | ALA | 4.13 | −1.43 | 0.00 | 0.00 | 5.55 |
| ** | 15 | ASP | −2.05 | −2.23 | −0.22 | 0.00 | 0.40 |
| * | 15 | GLU | −0.61 | −2.42 | −0.19 | 0.00 | 2.01 |
| * | 15 | HIS | 8.24 | −2.87 | −0.01 | 0.00 | 11.11 |
| * | 15 | HSP | 7.89 | −2.87 | 0.22 | 0.00 | 10.54 |
| * | 15 | LYS | 4.45 | −2.65 | 0.18 | 0.00 | 6.92 |
| * | 15 | ASN | −0.40 | −2.86 | 0.01 | 0.00 | 2.45 |
| ** | 15 | GLN | −1.29 | −2.45 | 0.01 | 0.00 | 1.15 |
| * | 15 | ARG | 0.02 | −2.55 | 0.20 | 0.00 | 2.36 |
| ** | 15 | SER | −1.36 | −1.64 | 0.00 | 0.00 | 0.27 |
| * | 15 | THR | 4.55 | −2.43 | 0.02 | 0.00 | 6.96 |
| | 22 | TRP | 18.45 | −5.92 | 0.00 | 0.00 | 24.37 |
| * | 22 | ALA | 4.20 | −1.41 | 0.00 | 0.00 | 5.61 |
| * | 22 | ASP | 0.36 | −2.04 | −0.31 | 0.00 | 2.71 |
| ** | 22 | GLU | −1.48 | −3.44 | −0.22 | 0.00 | 2.18 |
| * | 22 | HIS | 11.29 | 0.90 | −0.15 | 0.00 | 10.54 |
| * | 22 | HSP | 10.51 | 0.24 | −0.05 | 0.00 | 10.32 |
| * | 22 | LYS | 1.76 | −3.78 | 0.24 | 0.00 | 5.31 |
| * | 22 | ASN | 0.23 | −2.05 | −0.05 | 0.00 | 2.33 |
| ** | 22 | GLN | −2.43 | −3.44 | 0.01 | 0.00 | 1.00 |
| * | 22 | ARG | 0.66 | −3.42 | 0.23 | 0.00 | 3.84 |
| ** | 22 | SER | −1.24 | −1.58 | −0.01 | 0.00 | 0.35 |
| * | 22 | THR | 3.43 | −2.85 | 0.05 | 0.00 | 6.22 |
| | 28 | LEU | 2.83 | −5.56 | 0.00 | 0.00 | 8.40 |
| * | 28 | ALA | 2.61 | −1.61 | 0.00 | 0.00 | 4.21 |
| * | 28 | ASP | 1.55 | −3.49 | 0.01 | 0.00 | 5.03 |
| * | 28 | GLU | −1.66 | −3.82 | −0.04 | 0.00 | 2.20 |
| | 28 | HIS | 4.28 | −5.06 | 0.06 | 0.00 | 9.28 |
| | 28 | HSP | 5.23 | −4.96 | 0.04 | −0.73 | 10.88 |
| * | 28 | LYS | −0.87 | −4.43 | −0.01 | 0.00 | 3.57 |
| * | 28 | ASN | 0.72 | −3.46 | 0.04 | 0.00 | 4.14 |
| ** | 28 | GLN | −6.92 | −3.78 | −0.11 | −5.30 | 2.27 |
| | 28 | ARG | 3.10 | −6.28 | 0.21 | 0.00 | 9.17 |
| * | 28 | SER | 0.59 | −2.01 | −0.01 | 0.00 | 2.62 |
| | 28 | THR | 7.09 | −2.50 | 0.01 | 0.00 | 9.57 |
| | 30 | TYR | 13.74 | −3.59 | −0.05 | 0.00 | 17.38 |
| * | 30 | ALA | 10.72 | −0.88 | 0.00 | 0.00 | 11.60 |
| ** | 30 | ASP | 3.32 | −1.36 | −0.24 | 0.00 | 4.92 |
| * | 30 | GLU | 5.32 | −1.88 | −0.29 | 0.00 | 7.49 |
| * | 30 | HIS | 9.66 | −2.99 | −0.08 | 0.00 | 12.73 |
| * | 30 | HSP | 12.47 | −3.00 | 0.74 | 0.00 | 14.73 |
| * | 30 | LYS | 8.65 | −2.26 | 0.19 | 0.00 | 10.72 |
| ** | 30 | ASN | 2.78 | −1.37 | 0.01 | 0.00 | 4.15 |
| * | 30 | GLN | 4.45 | −1.89 | −0.01 | 0.00 | 6.35 |
| * | 30 | ARG | 7.17 | −1.90 | 0.15 | 0.00 | 8.93 |
| * | 30 | SER | 4.49 | −1.03 | −0.02 | 0.00 | 5.54 |
| * | 30 | THR | 7.17 | −1.69 | −0.02 | 0.00 | 8.88 |
| | 32 | LEU | 0.79 | −4.68 | 0.00 | 0.00 | 5.47 |
| ** | 32 | ALA | −0.14 | −1.52 | 0.00 | 0.00 | 1.38 |
| | 32 | ASP | 1.58 | −3.02 | −0.21 | 0.00 | 4.81 |
| * | 32 | GLU | 0.18 | −4.32 | −0.47 | 0.00 | 4.97 |
| * | 32 | HIS | −0.42 | −4.84 | −0.17 | 0.00 | 4.58 |
| ** | 32 | HSP | −0.93 | −4.84 | −0.22 | 0.00 | 4.13 |
| | 32 | LYS | 2.85 | −4.41 | 0.39 | 0.00 | 6.87 |
| | 32 | ASN | 3.94 | −3.09 | −0.04 | 0.00 | 7.06 |
| * | 32 | GLN | 0.22 | −4.00 | 0.01 | 0.00 | 4.21 |
| * | 32 | ARG | 0.95 | −4.74 | 0.36 | 0.00 | 5.33 |
| * | 32 | SER | 0.83 | −1.93 | 0.06 | 0.00 | 2.70 |
| | 32 | THR | 1.72 | −3.10 | 0.06 | 0.00 | 4.76 |
| | 36 | MET | 0.14 | −5.60 | 0.00 | 0.00 | 5.74 |
| | 36 | ALA | 0.38 | −1.86 | 0.00 | 0.00 | 2.24 |
| ** | 36 | ASP | −3.06 | −3.47 | 0.02 | −0.03 | 0.43 |
| ** | 36 | GLU | −3.53 | −3.34 | −0.05 | 0.00 | −0.14 |
| * | 36 | HIS | −0.84 | −5.33 | 0.03 | 0.00 | 4.46 |
| | 36 | HSP | 0.32 | −5.04 | −0.08 | 0.00 | 5.44 |
| ** | 36 | LYS | −3.76 | −4.99 | 0.00 | 0.00 | 1.22 |
| * | 36 | ASN | −1.09 | −3.53 | 0.00 | −0.05 | 2.48 |
| ** | 36 | GLN | −5.26 | −2.66 | −0.10 | −2.32 | −0.18 |
| * | 36 | ARG | −2.19 | −2.92 | 0.05 | 0.00 | 0.69 |
| * | 36 | SER | −2.41 | −2.27 | 0.02 | 0.00 | −0.17 |
| 2** | 36 | THR | −3.93 | −1.20 | 0.02 | 0.00 | −2.76 |
| | 47 | LEU | 1.86 | −6.08 | 0.00 | 0.00 | 7.94 |
| * | 47 | ALA | 0.52 | −2.11 | 0.00 | 0.00 | 2.62 |
| ** | 47 | ASP | −7.26 | −4.20 | −0.37 | −2.90 | 0.22 |
| * | 47 | GLU | −2.33 | −4.94 | 0.02 | 0.00 | 2.59 |
| | 47 | HIS | 217.36 | 213.11 | 0.09 | 0.00 | 4.16 |
| | 47 | HSP | 4313.02 | 4309.27 | −2.51 | 0.00 | 6.27 |
| ** | 47 | LYS | −5.22 | −5.97 | 0.01 | 0.00 | 0.74 |
| ** | 47 | ASN | −4.27 | −4.31 | −0.18 | −2.14 | 2.37 |
| * | 47 | GLN | −1.65 | −5.40 | −0.07 | −2.13 | 5.95 |
| * | 47 | ARG | −3.84 | −4.76 | −0.27 | −6.29 | 7.49 |
| * | 47 | SER | −1.23 | −2.64 | 0.03 | 0.00 | 1.37 |
| * | 47 | THR | −0.02 | −2.58 | 0.01 | 0.00 | 2.56 |
| | 92 | TYR | 3.84 | −5.11 | 0.01 | 0.00 | 8.95 |
| * | 92 | ALA | −1.94 | −1.95 | 0.00 | 0.00 | 0.01 |
| ** | 92 | ASP | −5.45 | −3.06 | −0.33 | −0.01 | −2.04 |
| ** | 92 | GLU | −5.14 | −3.67 | −0.08 | 0.00 | −1.40 |
| * | 92 | HIS | 3.04 | −4.25 | −0.04 | 0.00 | 7.33 |
| * | 92 | HSP | 2.94 | −4.25 | 0.28 | 0.00 | 6.91 |
| * | 92 | LYS | −1.75 | −3.96 | 0.00 | 0.00 | 2.21 |
| * | 92 | ASN | −3.30 | −3.13 | −0.12 | −0.03 | −0.02 |
| ** | 92 | GLN | −5.55 | −3.69 | 0.02 | 0.00 | −1.89 |
| * | 92 | ARG | −0.49 | −3.72 | 0.14 | 0.00 | 3.10 |
| ** | 92 | SER | −4.90 | −2.25 | −0.03 | 0.00 | −2.62 |
| | 92 | THR | 4.46 | 0.21 | 0.00 | 0.00 | 4.25 |
| | 111 | PHE | 29.59 | −2.42 | 0.00 | 0.00 | 32.01 |
| * | 111 | ALA | 15.98 | −0.76 | 0.00 | 0.00 | 16.74 |
| ** | 111 | ASP | 8.56 | −1.11 | 0.03 | 0.00 | 9.64 |
| * | 111 | GLU | 13.15 | −1.18 | −0.07 | 0.00 | 14.39 |
| * | 111 | HIS | 19.66 | −1.33 | 0.00 | 0.00 | 20.99 |
| * | 111 | HSP | 19.06 | −1.33 | −0.02 | 0.00 | 20.41 |
| * | 111 | LYS | 20.27 | −1.30 | 0.08 | 0.00 | 21.49 |
| ** | 111 | ASN | 7.32 | −1.10 | 0.00 | 0.00 | 8.41 |
| * | 111 | GLN | 11.91 | −1.18 | −0.03 | 0.00 | 13.12 |
| * | 111 | ARG | 15.55 | −1.25 | 0.02 | 0.00 | 16.78 |
| ** | 111 | SER | 9.49 | −0.86 | 0.01 | 0.00 | 10.34 |
| * | 111 | THR | 14.87 | −0.10 | −0.10 | −0.71 | 15.78 |
| | 116 | LEU | 4.71 | −3.66 | 0.00 | 0.00 | 8.37 |
| * | 116 | ALA | 1.74 | −1.32 | 0.00 | 0.00 | 3.06 |
| ** | 116 | ASP | −2.58 | −2.25 | −0.19 | 0.00 | −0.13 |
| * | 116 | GLU | −1.53 | −3.11 | −0.11 | 0.00 | 1.69 |
| | 116 | HIS | 7.67 | −3.22 | 0.11 | 0.00 | 10.78 |
| | 116 | HSP | 7.44 | −3.22 | 0.50 | 0.00 | 10.16 |
| * | 116 | LYS | 1.45 | −3.27 | 0.03 | 0.00 | 4.68 |
| ** | 116 | ASN | −2.54 | −2.29 | −0.05 | 0.00 | −0.20 |
| * | 116 | GLN | −1.95 | −3.13 | −0.01 | 0.00 | 1.18 |
| * | 116 | ARG | −1.05 | −3.53 | 0.29 | 0.00 | 2.18 |
| * | 116 | SER | −1.66 | −1.55 | −0.01 | 0.00 | −0.10 |
| * | 116 | THR | 1.59 | −1.87 | −0.01 | 0.00 | 3.47 |
| | 120 | LEU | 0.81 | −6.47 | 0.00 | 0.00 | 7.28 |
| | 120 | ALA | 2.03 | −1.44 | 0.00 | 0.00 | 3.46 |
| ** | 120 | ASP | −2.85 | −2.28 | −0.33 | 0.00 | −0.24 |
| | 120 | GLU | 1.19 | −2.64 | −0.16 | 0.00 | 3.99 |
| | 120 | HIS | 10.00 | −3.07 | 0.08 | 0.00 | 12.99 |
| | 120 | HSP | 9.96 | −2.91 | 0.20 | 0.00 | 12.68 |
| | 120 | LYS | 6.44 | −2.73 | 0.30 | 0.00 | 8.87 |
| * | 120 | ASN | −1.33 | −2.21 | −0.05 | 0.00 | 0.94 |
| * | 120 | GLN | 0.39 | −2.66 | 0.04 | 0.00 | 3.01 |
| | 120 | ARG | 4.28 | −2.64 | 0.23 | 0.00 | 6.69 |
| ** | 120 | SER | −2.59 | −1.64 | −0.05 | 0.00 | −0.90 |
| | 120 | THR | 3.04 | −3.74 | −0.01 | 0.00 | 6.80 |
| | 130 | LEU | −4.92 | −5.89 | 0.00 | 0.00 | 0.98 |
| | 130 | ALA | 0.46 | −1.57 | 0.00 | 0.00 | 2.03 |
| * | 130 | ASP | −4.43 | −2.75 | −0.13 | 0.00 | −1.55 |
| ** | 130 | GLU | −6.43 | −3.00 | −0.16 | 0.00 | −3.28 |
| | 130 | HIS | 0.41 | −4.27 | −0.03 | 0.00 | 4.71 |
| | 130 | HSP | 2.99 | −4.38 | 0.03 | 0.00 | 7.34 |
| * | 130 | LYS | −4.72 | −5.08 | 0.18 | 0.00 | 0.19 |
| * | 130 | ASN | −4.59 | −2.79 | 0.00 | 0.00 | −1.80 |
| ** | 130 | GLN | −6.62 | −4.38 | 0.01 | 0.00 | −2.25 |
| ** | 130 | ARG | −5.87 | −5.87 | −0.01 | −2.32 | 2.33 |
| | 130 | SER | −3.50 | −1.84 | 0.00 | 0.00 | −1.66 |
| | 130 | THR | −3.29 | −3.41 | 0.02 | 0.00 | 0.09 |
| | 148 | VAL | 6.65 | −3.33 | 0.00 | 0.00 | 9.98 |
| | 148 | ALA | 7.09 | −1.45 | 0.00 | 0.00 | 8.54 |
| ** | 148 | ASP | 0.64 | −2.35 | −0.29 | 0.00 | 3.28 |
| ** | 148 | GLU | 1.02 | −3.73 | −0.30 | 0.00 | 5.06 |
| | 148 | HIS | 7.65 | −3.09 | −0.04 | 0.00 | 10.79 |
| | 148 | HSP | 7.26 | −3.10 | 0.16 | 0.00 | 10.20 |
| * | 148 | LYS | 2.96 | −4.18 | 0.36 | 0.00 | 6.77 |
| * | 148 | ASN | 2.53 | −2.37 | −0.02 | 0.00 | 4.92 |
| * | 148 | GLN | 2.96 | −2.72 | 0.03 | 0.00 | 5.64 |
| ** | 148 | ARG | 1.86 | −3.88 | 0.34 | 0.00 | 5.40 |
| ** | 148 | SER | 1.08 | −1.68 | 0.00 | 0.00 | 2.77 |
| * | 148 | THR | 5.24 | −2.58 | 0.03 | 0.00 | 7.79 |
| | 155 | TYR | 6.95 | −4.80 | −0.01 | 0.00 | 11.76 |
| * | 155 | ALA | 4.11 | −1.52 | 0.00 | 0.00 | 5.63 |
| ** | 155 | ASP | −1.98 | −2.45 | −0.29 | 0.00 | 0.76 |
| * | 155 | GLU | −0.57 | −3.62 | −0.27 | 0.00 | 3.31 |
| | 155 | HIS | 8.86 | −3.52 | 0.01 | 0.00 | 12.37 |
| | 155 | HSP | 9.02 | −3.52 | 0.31 | 0.00 | 12.23 |
| * | 155 | LYS | 5.53 | −2.99 | 0.25 | 0.00 | 8.27 |
| * | 155 | ASN | 0.17 | −2.47 | −0.01 | 0.00 | 2.65 |
| ** | 155 | GLN | −1.50 | −3.63 | 0.00 | 0.00 | 2.13 |
| ** | 155 | ARG | 1.29 | −3.63 | 0.28 | 0.00 | 4.65 |
| * | 155 | SER | −0.82 | −1.77 | 0.01 | 0.00 | 0.94 |
| * | 155 | THR | 5.05 | −2.70 | 0.00 | 0.00 | 7.75 |

TABLE 10
Interferon kappa calculation results, exposed hydrophobic residues

| | # | AA | Total | vdW | Elec | Hbond | Solv |
|---|---|---|---|---|---|---|---|
| | 1 | LEU | 16.16 | −1.74 | 0.00 | 0.00 | 17.90 |
| * | 1 | ALA | 8.55 | −0.56 | 0.00 | 0.00 | 9.12 |
| * | 1 | ARG | 5.07 | −1.90 | −0.32 | 0.00 | 7.29 |
| * | 1 | ASN | 2.47 | −1.03 | 0.12 | 0.00 | 3.38 |
| ** | 1 | ASP | 0.82 | −1.11 | −0.05 | −3.98 | 5.96 |
| * | 1 | GLN | 2.37 | −1.39 | 0.03 | 0.00 | 3.73 |
| * | 1 | GLU | 3.52 | −1.14 | 0.22 | 0.00 | 4.45 |
| * | 1 | GLY | 2.79 | −0.09 | 0.00 | 0.00 | 2.88 |
| * | 1 | HIS | 10.39 | −1.90 | −0.15 | −2.54 | 14.97 |
| * | 1 | HSP | 9.14 | −1.90 | −1.03 | −2.53 | 14.61 |
| * | 1 | LYS | 7.37 | −0.82 | −0.27 | 0.00 | 8.46 |
| * | 1 | SER | 3.41 | −0.54 | 0.03 | 0.00 | 3.92 |
| * | 1 | THR | 6.26 | −1.13 | 0.03 | 0.00 | 7.37 |
| | 5 | LEU | 9.28 | −3.12 | 0.00 | 0.00 | 12.40 |
| * | 5 | ALA | 6.92 | −1.11 | 0.00 | 0.00 | 8.03 |
| * | 5 | ARG | 2.30 | −2.28 | 0.16 | 0.00 | 4.42 |
| ** | 5 | ASN | −1.00 | −1.73 | 0.02 | 0.00 | 0.71 |
| ** | 5 | ASP | −0.31 | −1.73 | −0.28 | 0.00 | 1.69 |
| * | 5 | GLN | 0.46 | −2.44 | 0.00 | 0.00 | 2.91 |
| * | 5 | GLU | 1.43 | −2.42 | −0.17 | 0.00 | 4.02 |
| * | 5 | GLY | 6.79 | −0.17 | 0.00 | 0.00 | 6.96 |
| * | 5 | HIS | 6.18 | −2.38 | −0.01 | 0.00 | 8.57 |
| * | 5 | HSP | 6.04 | −2.38 | 0.23 | 0.00 | 8.19 |
| * | 5 | LYS | 2.82 | −3.46 | 0.42 | −3.19 | 9.05 |
| * | 5 | SER | 1.03 | −1.26 | −0.01 | 0.00 | 2.29 |
| * | 5 | THR | 1.09 | −2.29 | −0.01 | 0.00 | 3.39 |
| | 8 | VAL | 5.07 | −3.35 | 0.00 | 0.00 | 8.42 |
| * | 8 | ALA | 5.02 | −1.40 | 0.00 | 0.00 | 6.43 |
| * | 8 | ARG | −0.04 | −3.23 | 0.36 | 0.00 | 2.83 |
| ** | 8 | ASN | −3.01 | −2.45 | −0.09 | −2.84 | 2.37 |
| * | 8 | ASP | −0.54 | −2.52 | −0.30 | 0.00 | 2.29 |
| ** | 8 | GLN | −2.05 | −2.96 | 0.04 | 0.00 | 0.88 |
| ** | 8 | GLU | −1.27 | −2.68 | −0.26 | 0.00 | 1.66 |
| * | 8 | GLY | 2.09 | −0.22 | 0.00 | 0.00 | 2.30 |
| * | 8 | HIS | 2.94 | −3.79 | 0.03 | 0.00 | 6.70 |
| * | 8 | HSP | 3.07 | −3.79 | 0.37 | 0.00 | 6.49 |
| * | 8 | LYS | 0.38 | −3.42 | 0.33 | 0.00 | 3.47 |
| * | 8 | SER | 0.32 | −1.69 | 0.00 | 0.00 | 2.01 |
| * | 8 | THR | 2.44 | −2.69 | 0.00 | 0.00 | 5.13 |
| | 15 | TRP | 2.66 | −6.08 | 0.00 | 0.00 | 8.74 |
| * | 15 | ALA | 2.27 | −1.39 | 0.00 | 0.00 | 3.66 |
| * | 15 | ARG | −0.49 | −3.53 | 0.41 | 0.00 | 2.63 |
| ** | 15 | ASN | −4.15 | −2.97 | 0.05 | −2.71 | 1.48 |
| ** | 15 | ASP | −3.09 | −2.99 | −0.43 | 0.00 | 0.32 |
| ** | 15 | GLN | −4.26 | −3.24 | −0.01 | 0.00 | −1.01 |
| ** | 15 | GLU | −3.94 | −3.19 | −0.36 | 0.00 | −0.37 |
| * | 15 | GLY | 1.98 | −0.30 | 0.00 | 0.00 | 2.28 |
| | 15 | HIS | 3.07 | −3.90 | 0.01 | 0.00 | 6.96 |
| | 15 | HSP | 3.13 | −3.88 | 0.42 | 0.00 | 6.59 |
| * | 15 | LYS | −0.64 | −2.80 | 0.43 | 0.00 | 1.73 |
| * | 15 | SER | −1.70 | −1.75 | −0.01 | 0.00 | 0.07 |
| | 15 | THR | 5.05 | −0.75 | 0.03 | 0.00 | 5.77 |
| | 18 | LEU | −7.96 | −6.28 | 0.00 | 0.00 | −1.69 |
| | 18 | ALA | −3.37 | −2.20 | 0.00 | 0.00 | −1.16 |
| | 18 | ARG | −3.90 | −5.75 | 0.36 | 0.00 | 1.48 |
| | 18 | ASN | −3.50 | −4.51 | 0.00 | 0.00 | 1.02 |
| | 18 | ASP | −5.98 | −4.64 | −0.35 | 0.00 | −0.99 |
| * | 18 | GLN | −7.59 | −4.63 | −0.01 | 0.00 | −2.95 |
| * | 18 | GLU | −8.87 | −5.82 | −0.43 | 0.00 | −2.61 |
| | 18 | GLY | 0.11 | −0.37 | 0.00 | 0.00 | 0.48 |
| | 18 | HIS | −0.92 | −4.87 | −0.02 | 0.00 | 3.96 |
| | 18 | HSP | 3.12 | −3.46 | 0.42 | 0.00 | 6.16 |
| * | 18 | LYS | −6.70 | −6.21 | 0.30 | 0.00 | −0.79 |
| | 18 | SER | −3.95 | −2.68 | 0.00 | 0.00 | −1.27 |
| | 18 | THR | −1.25 | −3.94 | 0.07 | 0.00 | 2.61 |
| | 28 | PHE | 18.32 | −4.71 | 0.00 | 0.00 | 23.02 |
| * | 28 | ALA | 5.85 | −1.85 | 0.00 | 0.00 | 7.69 |
| * | 28 | ARG | 3.35 | −3.31 | −0.03 | 0.00 | 6.69 |
| ** | 28 | ASN | −2.32 | −3.19 | −0.19 | −3.03 | 4.09 |
| * | 28 | ASP | 1.28 | −2.94 | 0.28 | 0.00 | 3.93 |
| * | 28 | GLN | 0.95 | −3.74 | −0.14 | −3.37 | 8.21 |
| * | 28 | GLU | 3.31 | −3.39 | 0.15 | 0.00 | 6.55 |
| * | 28 | GLY | 6.33 | −0.28 | 0.00 | 0.00 | 6.62 |
| * | 28 | HIS | 7.67 | −4.12 | 0.03 | 0.00 | 11.76 |
| * | 28 | HSP | 6.77 | −4.11 | −0.24 | 0.00 | 11.12 |
| * | 28 | LYS | 4.45 | −3.59 | −0.52 | −5.05 | 13.61 |
| * | 28 | SER | 1.76 | −2.16 | 0.01 | 0.00 | 3.91 |
| * | 28 | THR | 9.75 | 2.16 | 0.00 | 0.00 | 7.60 |
| | 30 | VAL | 10.27 | −2.35 | 0.00 | 0.00 | 12.62 |
| * | 30 | ALA | 6.08 | −0.92 | 0.00 | 0.00 | 7.00 |
| * | 30 | ARG | 2.49 | −2.42 | 0.06 | 0.00 | 4.85 |
| * | 30 | ASN | 0.13 | −1.83 | 0.00 | 0.00 | 1.97 |
| * | 30 | ASP | 1.13 | −1.82 | 0.04 | 0.00 | 2.91 |
| ** | 30 | GLN | −0.65 | −1.87 | −0.02 | 0.00 | 1.24 |
| * | 30 | GLU | 0.68 | −1.87 | 0.01 | 0.00 | 2.54 |
| * | 30 | GLY | 2.71 | −0.16 | 0.00 | 0.00 | 2.87 |
| * | 30 | HIS | 7.83 | −3.68 | −0.01 | 0.00 | 11.52 |
| * | 30 | HSP | 7.87 | −3.56 | −0.13 | 0.00 | 11.56 |
| * | 30 | LYS | 5.43 | −3.08 | 0.01 | 0.00 | 8.51 |
| * | 30 | SER | 1.64 | −1.15 | 0.00 | 0.00 | 2.78 |
| * | 30 | THR | 5.28 | −1.93 | 0.01 | 0.00 | 7.20 |
| | 33 | LEU | 8.89 | −3.10 | 0.00 | 0.00 | 12.00 |
| * | 33 | ALA | 5.67 | −0.99 | 0.00 | 0.00 | 6.67 |
| * | 33 | ARG | −0.88 | −2.82 | −0.07 | 0.00 | 2.01 |
| ** | 33 | ASN | −1.09 | −1.86 | 0.00 | 0.00 | 0.78 |
| * | 33 | ASP | 0.12 | −1.86 | 0.12 | 0.00 | 1.86 |
| ** | 33 | GLN | −3.13 | −2.90 | −0.09 | −2.65 | 2.51 |
| * | 33 | GLU | −0.44 | −2.85 | 0.16 | 0.00 | 2.24 |
| * | 33 | GLY | 2.91 | −0.15 | 0.00 | 0.00 | 3.07 |
| * | 33 | HIS | 6.16 | −2.83 | 0.01 | 0.00 | 8.98 |
| * | 33 | HSP | 5.57 | −2.83 | −0.12 | 0.00 | 8.51 |
| * | 33 | LYS | 1.75 | −2.89 | −0.09 | 0.00 | 4.73 |
| * | 33 | SER | 0.39 | −1.19 | 0.01 | 0.00 | 1.58 |
| * | 33 | THR | 1.15 | −2.27 | −0.01 | 0.00 | 3.42 |
| | 37 | ILE | 0.71 | −5.77 | 0.00 | 0.00 | 6.48 |
| | 37 | ALA | 3.26 | −1.68 | 0.00 | 0.00 | 4.94 |
| * | 37 | ARG | −1.63 | −2.56 | −0.39 | −5.88 | 7.21 |
| * | 37 | ASN | −1.24 | −3.19 | 0.03 | 0.00 | 1.92 |
| * | 37 | ASP | −3.15 | −2.98 | 0.23 | −0.10 | −0.30 |
| ** | 37 | GLN | −6.08 | −3.22 | −0.06 | −4.23 | 1.44 |
| * | 37 | GLU | −2.78 | −3.25 | 0.27 | 0.00 | 0.19 |
| | 37 | GLY | 2.71 | −0.21 | 0.00 | 0.00 | 2.92 |
| | 37 | HIS | 2.18 | −5.14 | 0.01 | 0.00 | 7.30 |
| | 37 | HSP | 2.77 | −4.28 | −0.34 | −1.12 | 8.51 |
| * | 37 | LYS | −1.72 | −4.15 | −0.21 | 0.00 | 2.64 |
| * | 37 | SER | −0.42 | −1.99 | 0.01 | 0.00 | 1.55 |
| ** | 37 | THR | −4.92 | −4.32 | 0.01 | 0.00 | −0.62 |
| | 46 | LEU | 0.03 | −4.37 | 0.00 | 0.00 | 4.40 |
| * | 46 | ALA | −2.83 | −1.86 | 0.00 | 0.00 | −0.97 |
| ** | 46 | ARG | −5.84 | −4.27 | −0.18 | −2.39 | 1.00 |
| * | 46 | ASN | −4.07 | −3.26 | 0.00 | 0.00 | −0.81 |
| ** | 46 | ASP | −6.38 | −3.22 | −0.25 | 0.00 | −2.92 |
| ** | 46 | GLN | −7.53 | −3.68 | 0.01 | 0.00 | −3.86 |
| ** | 46 | GLU | −7.16 | −3.55 | −0.12 | 0.00 | −3.48 |
| * | 46 | GLY | −0.53 | −0.26 | 0.00 | 0.00 | −0.27 |
| | 46 | HIS | 0.17 | −4.16 | −0.02 | 0.00 | 4.35 |
| * | 46 | HSP | −0.20 | −4.15 | 0.17 | 0.00 | 3.78 |
| * | 46 | LYS | −3.15 | −3.48 | 0.15 | 0.00 | 0.19 |
| ** | 46 | SER | −5.21 | −2.19 | 0.01 | 0.00 | −3.03 |
| * | 46 | THR | −0.91 | 1.44 | 0.01 | 0.00 | −2.37 |
| | 48 | TYR | −3.30 | −5.42 | 0.01 | 0.00 | 2.10 |
| | 48 | ALA | −1.88 | −1.89 | 0.00 | 0.00 | 0.01 |
| * | 48 | ARG | −5.36 | −5.53 | −0.11 | 0.00 | 0.28 |
| | 48 | ASN | −2.23 | −3.76 | −0.03 | 0.00 | 1.55 |
| ** | 48 | ASP | −9.47 | −3.96 | 0.00 | −2.99 | −2.52 |
| * | 48 | GLN | −7.50 | −4.51 | −0.11 | −2.67 | −0.22 |
| ** | 48 | GLU | −9.11 | −4.52 | −0.05 | −2.71 | −1.83 |
| | 48 | GLY | 1.29 | −0.24 | 0.00 | 0.00 | 1.52 |
| | 48 | HIS | −1.45 | −5.38 | −0.03 | 0.00 | 3.96 |
| | 48 | HSP | −2.14 | −5.37 | −0.15 | 0.00 | 3.37 |
| * | 48 | LYS | −5.37 | −4.29 | −0.11 | 0.00 | −0.96 |
| | 48 | SER | −3.16 | −2.27 | −0.01 | 0.00 | −0.88 |
| * | 48 | THR | −4.68 | −1.54 | −0.01 | 0.00 | −3.13 |
| | 52 | MET | 12.92 | −3.56 | 0.00 | 0.00 | 16.48 |
| * | 52 | ALA | 5.97 | −1.54 | 0.00 | 0.00 | 7.51 |
| * | 52 | ARG | 3.75 | −2.96 | 0.15 | 0.00 | 6.56 |
| ** | 52 | ASN | −1.71 | −1.11 | −0.27 | −5.77 | 5.43 |
| ** | 52 | ASP | −1.46 | −1.59 | −1.25 | −3.93 | 5.32 |
| * | 52 | GLN | 1.34 | −3.03 | −0.07 | 0.00 | 4.44 |
| * | 52 | GLU | 2.17 | −2.98 | −0.28 | 0.00 | 5.43 |
| * | 52 | GLY | 4.74 | −0.23 | 0.00 | 0.00 | 4.97 |
| * | 52 | HIS | 7.79 | −2.91 | −0.28 | −3.46 | 14.44 |
| * | 52 | HSP | 6.75 | −2.89 | −0.70 | −3.48 | 13.82 |
| * | 52 | LYS | 6.71 | −3.15 | 0.16 | 0.00 | 9.70 |
| * | 52 | SER | 0.84 | −1.76 | 0.04 | 0.00 | 2.56 |
| * | 52 | THR | 5.25 | −1.27 | 0.04 | 0.00 | 6.48 |
| | 65 | LEU | −2.31 | −4.75 | 0.00 | 0.00 | 2.44 |
| | 65 | ALA | −1.88 | −1.76 | 0.00 | 0.00 | −0.12 |
| * | 65 | ARG | −3.62 | −4.35 | −0.05 | 0.00 | 0.79 |
| * | 65 | ASN | −2.88 | −3.75 | 0.01 | 0.00 | 0.86 |
| * | 65 | ASP | −4.97 | −3.88 | 0.30 | 0.00 | −1.39 |
| ** | 65 | GLN | −6.92 | −4.78 | 0.03 | 0.00 | −2.18 |
| ** | 65 | GLU | −6.66 | −4.91 | 0.23 | 0.00 | −1.98 |
| | 65 | GLY | 0.31 | −0.25 | 0.00 | 0.00 | 0.56 |
| | 65 | HIS | 11.96 | 10.19 | 0.01 | 0.00 | 1.75 |
| | 65 | HSP | 13.91 | 8.82 | 0.17 | 0.00 | 4.91 |
| * | 65 | LYS | −3.12 | −4.48 | −0.18 | 0.00 | 1.54 |
| * | 65 | SER | −3.53 | −2.15 | 0.01 | 0.00 | −1.39 |
| * | 65 | THR | −4.25 | −3.45 | −0.02 | 0.00 | −0.78 |
| | 68 | PHE | −5.87 | −7.03 | 0.00 | 0.00 | 1.16 |
| | 68 | ALA | −3.75 | −2.01 | 0.00 | 0.00 | −1.74 |
| * | 68 | ARG | −6.84 | −5.85 | −0.53 | 0.00 | −0.46 |
| | 68 | ASN | −4.99 | −4.40 | −0.04 | 0.00 | −0.55 |
| * | 68 | ASP | −6.55 | −3.87 | 0.34 | 0.00 | −3.02 |
| * | 68 | GLN | −8.01 | −5.42 | −0.02 | 0.00 | −2.56 |
| ** | 68 | GLU | −9.36 | −5.40 | 0.34 | 0.00 | −4.30 |
| | 68 | GLY | −0.85 | −0.30 | 0.00 | 0.00 | −0.54 |
| * | 68 | HIS | −6.00 | −6.05 | 0.04 | 0.00 | 0.02 |
| * | 68 | HSP | −6.74 | −5.97 | −0.34 | 0.00 | −0.42 |
| ** | 68 | LYS | −9.96 | −5.89 | −0.41 | 0.00 | −3.66 |
| | 68 | SER | −3.46 | −2.41 | −0.03 | 0.00 | −1.02 |
| | 68 | THR | −2.31 | −3.42 | −0.14 | 0.00 | 1.25 |
| | 76 | PHE | 17.46 | −4.29 | 0.00 | 0.00 | 21.75 |
| * | 76 | ALA | 6.77 | −1.11 | 0.00 | 0.00 | 7.88 |
| * | 76 | ARG | 3.07 | −2.50 | −0.10 | 0.00 | 5.67 |
| ** | 76 | ASN | −1.69 | −1.48 | −0.15 | −2.30 | 2.24 |
| ** | 76 | ASP | −0.22 | −1.71 | 0.06 | 0.00 | 1.43 |
| * | 76 | GLN | 1.69 | −2.19 | −0.04 | 0.00 | 3.93 |
| * | 76 | GLU | 2.66 | −2.09 | 0.09 | 0.00 | 4.65 |
| * | 76 | GLY | 6.19 | −0.15 | 0.00 | 0.00 | 6.35 |
| * | 76 | HIS | 9.14 | −3.17 | 0.06 | 0.00 | 12.25 |
| * | 76 | HSP | 8.48 | −3.17 | −0.34 | 0.00 | 11.99 |
| * | 76 | LYS | 8.39 | −2.70 | −0.15 | 0.00 | 11.24 |
| * | 76 | SER | 0.59 | −1.28 | −0.02 | 0.00 | 1.89 |
| * | 76 | THR | 2.57 | −2.46 | −0.02 | 0.00 | 5.05 |
| | 78 | TYR | 6.54 | −5.49 | −0.04 | 0.00 | 12.07 |
| | 78 | ALA | 7.63 | −1.15 | 0.00 | 0.00 | 8.79 |
| * | 78 | ARG | 4.88 | −2.52 | −0.07 | 0.00 | 7.47 |
| * | 78 | ASN | 3.23 | −2.44 | −0.02 | 0.00 | 5.69 |
| * | 78 | ASP | 3.05 | −2.26 | 0.07 | −0.94 | 6.18 |
| ** | 78 | GLN | 1.98 | −2.21 | −0.04 | 0.00 | 4.23 |
| ** | 78 | GLU | 1.67 | −2.22 | −0.02 | 0.00 | 3.91 |
| | 78 | GLY | 6.81 | −0.14 | 0.00 | 0.00 | 6.96 |
| * | 78 | HIS | 5.82 | −6.20 | −0.02 | 0.00 | 12.03 |
| * | 78 | HSP | 3.01 | −6.07 | −0.46 | −2.67 | 12.22 |
| * | 78 | LYS | 4.97 | −3.96 | −0.48 | 0.00 | 9.41 |
| * | 78 | SER | 3.33 | −1.23 | −0.12 | −5.35 | 10.03 |
| * | 78 | THR | 2.95 | −1.98 | −0.12 | −5.18 | 10.22 |
| | 79 | TRP | 10.75 | −4.92 | 0.01 | 0.00 | 15.65 |
| * | 79 | ALA | 3.38 | −1.21 | 0.00 | 0.00 | 4.59 |
| * | 79 | ARG | 0.30 | −2.70 | −0.07 | 0.00 | 3.06 |
| ** | 79 | ASN | −1.20 | −2.37 | 0.13 | 0.00 | 1.04 |
| * | 79 | ASP | −0.65 | −2.21 | 0.26 | 0.00 | 1.31 |
| ** | 79 | GLN | −2.65 | −2.77 | −0.10 | −7.46 | 7.69 |
| * | 79 | GLU | 0.31 | −2.79 | 0.14 | 0.00 | 2.96 |
| * | 79 | GLY | 1.45 | −0.20 | 0.00 | 0.00 | 1.66 |
| * | 79 | HIS | 6.19 | −2.99 | 0.04 | 0.00 | 9.15 |
| * | 79 | HSP | 5.75 | −2.99 | −0.17 | 0.00 | 8.90 |
| * | 79 | LYS | 1.55 | −3.33 | −0.19 | 0.00 | 5.07 |
| * | 79 | SER | −0.73 | −1.40 | 0.00 | 0.00 | 0.67 |
| * | 79 | THR | 3.74 | −2.24 | −0.05 | −0.02 | 6.05 |
| | 89 | ILE | 5.42 | −4.08 | 0.00 | 0.00 | 9.50 |
| * | 89 | ALA | 3.77 | −1.15 | 0.00 | 0.00 | 4.92 |
| * | 89 | ARG | −1.59 | −4.17 | 0.11 | 0.00 | 2.48 |
| ** | 89 | ASN | −3.80 | −1.93 | 0.02 | 0.00 | −1.89 |
| ** | 89 | ASP | −3.01 | −1.82 | 0.08 | 0.00 | −1.26 |
| * | 89 | GLN | −1.06 | −2.39 | 0.10 | 0.00 | 1.23 |
| * | 89 | GLU | −0.26 | −2.18 | −0.25 | 0.00 | 2.17 |
| * | 89 | GLY | 3.72 | −0.17 | 0.00 | 0.00 | 3.89 |
| * | 89 | HIS | 4.04 | −2.39 | −0.03 | 0.00 | 6.46 |
| * | 89 | HSP | 3.42 | −2.39 | −0.14 | 0.00 | 5.96 |
| * | 89 | LYS | 3.92 | −2.39 | 0.08 | 0.00 | 6.22 |
| * | 89 | SER | −1.60 | −1.33 | 0.04 | 0.00 | −0.31 |
| * | 89 | THR | −1.68 | −2.51 | 0.04 | 0.00 | 0.79 |
| | 97 | TYR | −1.92 | −5.22 | −0.02 | 0.00 | 3.32 |
| | 97 | ALA | 0.39 | −1.49 | 0.00 | 0.00 | 1.87 |
| ** | 97 | ARG | −3.91 | −4.23 | −0.68 | −3.13 | 4.13 |
| | 97 | ASN | −1.28 | −2.95 | 0.10 | 0.00 | 1.56 |
| | 97 | ASP | −1.03 | −2.50 | 0.18 | 0.00 | 1.29 |
| * | 97 | GLN | −2.98 | −3.34 | 0.02 | 0.00 | 0.35 |
| * | 97 | GLU | −2.53 | −3.45 | 0.21 | 0.00 | 0.71 |
| | 97 | GLY | 2.13 | −0.21 | 0.00 | 0.00 | 2.33 |
| | 97 | HIS | 1.22 | −4.20 | 0.01 | 0.00 | 5.41 |
| | 97 | HSP | 0.98 | −4.21 | 0.16 | 0.00 | 5.04 |
| | 97 | LYS | −0.50 | −4.16 | −0.11 | 0.00 | 3.77 |
| | 97 | SER | 0.18 | −1.76 | −0.06 | 0.00 | 2.01 |
| ** | 97 | THR | −3.47 | −3.33 | −0.03 | 0.00 | −0.12 |
| | 112 | MET | 0.07 | −5.90 | 0.00 | 0.00 | 5.97 |
| | 112 | ALA | 3.69 | −1.52 | 0.00 | 0.00 | 5.21 |
| ** | 112 | ARG | −3.11 | −4.06 | −0.40 | −2.39 | 3.74 |
| ** | 112 | ASN | −2.04 | −2.63 | 0.01 | 0.00 | 0.58 |
| * | 112 | ASP | −1.23 | −2.33 | 0.50 | 0.00 | 0.61 |
| * | 112 | GLN | −1.40 | −2.90 | 0.09 | 0.00 | 1.42 |
| * | 112 | GLU | −1.83 | −2.95 | 0.47 | 0.00 | 0.65 |
| | 112 | GLY | 2.47 | −0.19 | 0.00 | 0.00 | 2.66 |
| | 112 | HIS | 1.58 | −4.34 | 0.02 | 0.00 | 5.90 |
| | 112 | HSP | 1.55 | −4.36 | −0.56 | 0.00 | 6.48 |
| ** | 112 | LYS | −2.09 | −3.70 | −0.37 | 0.00 | 1.99 |
| * | 112 | SER | −0.70 | −1.75 | −0.01 | 0.00 | 1.07 |
| * | 112 | THR | −0.57 | −2.95 | −0.01 | 0.00 | 2.39 |
| | 115 | MET | 20.53 | −1.89 | 0.00 | 0.00 | 22.43 |
| * | 115 | ALA | 11.10 | −0.75 | 0.00 | 0.00 | 11.85 |
| * | 115 | ARG | 8.78 | −1.98 | −0.22 | 0.00 | 10.97 |
| ** | 115 | ASN | 3.56 | −1.30 | 0.01 | 0.00 | 4.87 |
| ** | 115 | ASP | 4.09 | −0.30 | −0.30 | −2.86 | 7.55 |
| * | 115 | GLN | 6.25 | −1.40 | −0.02 | 0.00 | 7.67 |
| * | 115 | GLU | 7.28 | −1.41 | 0.17 | 0.00 | 8.52 |
| ** | 115 | GLY | 4.47 | −0.15 | 0.00 | 0.00 | 4.63 |
| * | 115 | HIS | 14.96 | −1.92 | 0.02 | 0.00 | 16.86 |
| * | 115 | HSP | 14.25 | −1.92 | −0.20 | 0.00 | 16.37 |
| * | 115 | LYS | 11.59 | −2.01 | −0.21 | 0.00 | 13.81 |
| ** | 115 | SER | 4.62 | −0.91 | 0.00 | 0.00 | 5.53 |
| * | 115 | THR | 11.38 | 0.32 | 0.00 | 0.00 | 11.06 |
| | 120 | MET | 14.72 | −3.42 | 0.00 | 0.00 | 18.15 |
| * | 120 | ALA | 10.26 | −0.70 | 0.00 | 0.00 | 10.96 |
| * | 120 | ARG | 4.52 | −2.66 | −0.24 | 0.00 | 7.42 |
| ** | 120 | ASN | 2.06 | −1.28 | −0.02 | 0.00 | 3.36 |
| ** | 120 | ASP | 3.57 | −1.28 | 0.24 | 0.00 | 4.61 |
| ** | 120 | GLN | 3.28 | −1.52 | 0.01 | 0.00 | 4.79 |
| * | 120 | GLU | 4.92 | −1.64 | 0.32 | 0.00 | 6.23 |
| * | 120 | GLY | 6.29 | −0.11 | 0.00 | 0.00 | 6.41 |
| * | 120 | HIS | 10.39 | −2.74 | −0.03 | 0.00 | 13.16 |
| * | 120 | HSP | 9.47 | −2.75 | −0.48 | 0.00 | 12.70 |
| * | 120 | LYS | 7.88 | −2.63 | −0.26 | 0.00 | 10.77 |
| * | 120 | SER | 4.15 | −0.85 | 0.02 | 0.00 | 4.98 |
| * | 120 | THR | 8.44 | −1.54 | 0.00 | 0.00 | 9.99 |
| | 127 | VAL | 7.26 | 8.43 | 0.00 | 0.00 | −1.17 |
| ** | 127 | ALA | −3.43 | −1.35 | 0.00 | 0.00 | −2.09 |
| * | 127 | ARG | 0.00 | −7.82 | −0.88 | 0.00 | 8.70 |
| ** | 127 | ASN | −4.70 | −3.66 | −0.13 | −4.04 | 3.13 |
| ** | 127 | ASP | −6.95 | −3.82 | 0.68 | −3.10 | −0.71 |
| * | 127 | GLN | −0.81 | −5.91 | −0.07 | −0.29 | 5.46 |
| ** | 127 | GLU | −3.83 | −5.90 | 0.78 | 0.00 | 1.29 |
| ** | 127 | GLY | −2.85 | −0.30 | 0.00 | 0.00 | −2.55 |
| | 127 | HIS | 16.59 | 12.31 | −0.12 | 0.00 | 4.41 |
| | 127 | HSP | 19.54 | 14.09 | −1.04 | 0.00 | 6.50 |
| * | 127 | LYS | −1.30 | −4.78 | −0.07 | 0.00 | 3.56 |
| * | 127 | SER | −0.99 | −2.21 | −0.04 | 0.00 | 1.26 |
| ** | 127 | THR | −3.15 | −4.29 | −0.04 | 0.00 | 1.17 |
| | 133 | LEU | 9.92 | −3.97 | 0.00 | 0.00 | 13.89 |
| * | 133 | ALA | 8.39 | −0.97 | 0.00 | 0.00 | 9.35 |
| * | 133 | ARG | 3.29 | −3.25 | −0.18 | 0.00 | 6.72 |
| * | 133 | ASN | 2.32 | −1.71 | −0.19 | 0.00 | 4.22 |
| * | 133 | ASP | 3.00 | −1.70 | −0.27 | 0.00 | 4.97 |
| ** | 133 | GLN | −2.05 | −2.51 | −0.14 | −5.10 | 5.69 |
| * | 133 | GLU | 2.24 | −3.06 | 0.42 | 0.00 | 4.88 |
| * | 133 | GLY | 2.12 | −0.15 | 0.00 | 0.00 | 2.27 |
| * | 133 | HIS | 9.18 | −2.46 | 0.01 | 0.00 | 11.64 |
| * | 133 | HSP | 9.02 | −2.47 | 0.30 | 0.00 | 11.19 |
| * | 133 | LYS | 3.76 | −3.26 | −0.26 | 0.00 | 7.28 |
| * | 133 | SER | 3.26 | −1.17 | −0.02 | 0.00 | 4.45 |
| * | 133 | THR | 4.07 | −2.42 | −0.04 | 0.00 | 6.53 |
| | 151 | TYR | −2.01 | −5.96 | −0.20 | −2.23 | 6.37 |
| | 151 | ALA | 2.45 | −1.62 | 0.00 | 0.00 | 4.07 |
| * | 151 | ARG | −2.32 | −3.34 | 0.09 | 0.00 | 0.94 |
| | 151 | ASN | 0.06 | −3.31 | 0.03 | 0.00 | 3.34 |
| | 151 | ASP | −1.42 | −2.87 | 0.05 | 0.00 | 1.40 |
| ** | 151 | GLN | −3.98 | −4.25 | 0.03 | 0.00 | 0.24 |
| ** | 151 | GLU | −4.41 | −4.75 | −0.09 | 0.00 | 0.43 |
| | 151 | GLY | 0.89 | −0.23 | 0.00 | 0.00 | 1.12 |
| ** | 151 | HIS | −3.72 | −5.32 | 0.02 | 0.00 | 1.58 |
| | 151 | HSP | −1.50 | −5.39 | 0.06 | 0.00 | 3.83 |
| | 151 | LYS | −1.43 | −4.88 | 0.21 | 0.00 | 3.24 |
| | 151 | SER | 0.50 | −2.12 | −0.03 | −2.79 | 5.44 |
| | 151 | THR | −0.98 | −3.30 | 0.02 | 0.00 | 2.30 |
| | 161 | VAL | −2.90 | −4.54 | 0.00 | 0.00 | 1.64 |
| | 161 | ALA | −1.30 | −1.78 | 0.00 | 0.00 | 0.48 |
| * | 161 | ARG | −5.02 | −4.50 | 0.12 | 0.00 | −0.63 |
| * | 161 | ASN | −3.65 | −3.44 | −0.21 | −1.47 | 1.46 |
| ** | 161 | ASP | −6.06 | −3.46 | −0.40 | 0.00 | −2.21 |
| * | 161 | GLN | −4.93 | −4.30 | −0.01 | 0.00 | −0.62 |
| ** | 161 | GLU | −7.22 | −4.29 | −0.28 | 0.00 | −2.66 |
| | 161 | GLY | −1.08 | −0.25 | 0.00 | 0.00 | −0.83 |
| | 161 | HIS | −1.44 | −4.70 | 0.22 | 0.00 | 3.04 |
| | 161 | HSP | −1.34 | −4.71 | 0.85 | 0.00 | 2.51 |
| * | 161 | LYS | −4.79 | −4.47 | 0.14 | 0.00 | −0.45 |
| * | 161 | SER | −2.99 | −2.12 | −0.03 | 0.00 | −0.84 |
| | 161 | THR | −0.47 | −3.87 | −0.03 | 0.00 | 3.42 |
| | 168 | TYR | 1.50 | −7.16 | −0.05 | 0.00 | 8.71 |
| | 168 | ALA | 1.77 | −1.79 | 0.00 | 0.00 | 3.56 |
| * | 168 | ARG | −0.38 | −4.14 | 0.40 | 0.00 | 3.37 |
| ** | 168 | ASN | −1.76 | −3.23 | −0.07 | −2.62 | 4.16 |
| ** | 168 | ASP | −2.08 | −3.56 | −0.38 | 0.00 | 1.85 |
| ** | 168 | GLN | −1.72 | −3.90 | −0.01 | 0.00 | 2.19 |
| ** | 168 | GLU | −1.52 | −3.79 | −0.36 | 0.00 | 2.62 |
| | 168 | GLY | 1.91 | −0.28 | 0.00 | 0.00 | 2.18 |
| | 168 | HIS | 2.66 | −5.84 | 0.00 | 0.00 | 8.51 |
| | 168 | HSP | 5.46 | −5.83 | 0.59 | 0.00 | 10.70 |
| | 168 | LYS | 2.36 | −4.49 | 0.38 | 0.00 | 6.48 |
| * | 168 | SER | −0.98 | −2.17 | −0.01 | 0.00 | 1.20 |
| * | 168 | THR | 1.15 | −3.18 | −0.01 | 0.00 | 4.34 |
| | 171 | TYR | 1.43 | −4.26 | −0.04 | 0.00 | 5.73 |
| * | 171 | ALA | −0.78 | −1.66 | 0.00 | 0.00 | 0.87 |
| * | 171 | ARG | −4.70 | −3.96 | 0.36 | 0.00 | −1.10 |
| * | 171 | ASN | −3.30 | −2.81 | −0.01 | 0.00 | −0.47 |
| ** | 171 | ASP | −5.70 | −2.80 | −0.41 | 0.00 | −2.49 |
| ** | 171 | GLN | −6.16 | −3.14 | 0.01 | 0.00 | −3.03 |
| ** | 171 | GLU | −6.10 | −4.42 | −0.32 | 0.00 | −1.35 |
| * | 171 | GLY | 0.09 | −0.22 | 0.00 | 0.00 | 0.31 |
| * | 171 | HIS | −0.40 | −5.05 | −0.06 | −0.38 | 5.09 |
| * | 171 | HSP | 1.13 | −4.02 | 0.46 | 0.00 | 4.69 |
| * | 171 | LYS | −3.45 | −5.26 | 0.43 | 0.00 | 1.38 |
| ** | 171 | SER | −4.54 | −1.92 | 0.00 | 0.00 | −2.62 |
| * | 171 | THR | −2.12 | −2.78 | 0.00 | 0.00 | 0.66 |

Next, we simultaneously designed sets of exposed hydrophobic residues that are located close to each other in space. These calculations were performed to account for coupling between interacting positions. As before, sets of residues were considered to be compatible with interferon structure if their energy was similar to or more favorable than the energy of the wild type residues at that set of positions. The most preferred sets of residues are those with the most favorable energies.

Calculation were performed on the following clusters of exposed hydrophobic residues in interferon beta: 5 and 8; 15 and 155; 22 and 148; 22, 30, 32, and 36; and 116 and 120. Results of the cluster calculations for interferon beta are given in the table below:

TABLE 11
Interferon beta calculation results, exposed hydrophobic clusters

| # | Most preferred | preferred |
|---|---|---|
| 5 | T | S, N, K, E |
| 8 | E | D, N, Q, S, R |
| 15 | D | |
| 22 | E | K, D, S, Q, R, N |
| 28 | Q | K |
| 30 | D | T, S, N, E |
| 32 | S | E |
| 36 | T | K, E |
| 116 | T | K, S, N, D, H, E |
| 120 | R | D, K, E, T, S |
| 148 | E | |
| 155 | D | E, N, S, Q |

Finally, we reconciled the results of the PDA® technology calculations and the sequence alignment data for interferon kappa. The most preferred polar substitution for each exposed hydrophobic residue was defined to be the residue with the highest normalized frequency of occurance, among the set of polar residues with favorable energies in the PDA® technology calculations. The most preferred substitutions are: V8N, W15R, V30R, 137N, Y48Q, F76S, I89T, Y97D, M112T, M115G, V161A, Y168S, and Y171T. In the case of Y97D and V161A, the replacements have slightly less favorable energies than the wild type hydrophobic residue. However, since the energy difference is only slight and the alternate residues are frequently observed in other interferons, it is likely that these substitutions are structurally and functionally suitable.

A few of these substitutions are close in sequence to other exposed hydrophobic residues. As a result, it was possible to test the effect of altering a small number of additional residues without increasing the overall library complexity. Preferred polar residues for these additional exposed hydrophobic residues were selected for favorable PDA® technology energies or high normalized frequency in other interferons; the most preferred substitutions are: L5Q, F28Q, M52N, Y78A, and L133Q.

### Example 6

### Identification of Suitable Replacements for Dimer Interface Residues

PDA® technology calculations were performed to identify residues that form favorable intermolecular interactions in the interferon-beta dimer. Each of the residues identified as dimer interface residues was considered. The interaction energy between each dimer interface residue in chain A and each dimer interface residue in chain B was calculated using a force field describing van der Waals interactions, electrostatics, hydrogen bonds, and salvation. The residues were all held fixed in the crystallographically observed conformations. Half- interaction energies are as shown below; the energies are symmetric and the total interaction energy is twice the value shown.

TABLE 12
Interactions across the interferon-beta dimer interface.

| | Glu | Glu | Gln | Leu | Gln | Gln | Gln | Arg | Leu | Met | Leu | His | Arg |
|---|---|---|---|---|---|---|---|---|---|---|---|---|---|
| | 42 A | 43 A | 46 A | 47 A | 48 A | 49 A | 51 A | 113 A | 116 A | 117 A | 120 A | 121 A | 124 A |
| MET 1 B | 0.0 | 0.0 | 0.0 | 0.0 | 0.0 | 0.0 | −1.0 | −1.4 | −0.1 | 0.0 | 0.0 | 0.0 | 0.0 |
| SER 2 B | 0.0 | 0.0 | 0.0 | 0.0 | 0.0 | 0.0 | 0.0 | −1.8 | −2.4 | 0.0 | 0.0 | 0.0 | 0.0 |
| TYR 3 B | 0.0 | 0.0 | 0.0 | 0.0 | 0.0 | 0.0 | 0.0 | 0.6 | 0.0 | 0.0 | 0.0 | 0.0 | 0.0 |
| ASN 4 B | 0.0 | 0.0 | 0.0 | −0.2 | 0.0 | −1.4 | 1.9 | 0.0 | 0.0 | 0.0 | 0.0 | 0.0 | 0.0 |
| LEU 5 B | 0.0 | −2.2 | 0.0 | −2.0 | 0.0 | 0.0 | 0.0 | 0.0 | −1.5 | −2.5 | −1.0 | −1.0 | 0.0 |
| LEU 6 B | 0.0 | 0.0 | 0.0 | 0.0 | 0.0 | 0.0 | 0.0 | −0.2 | −0.7 | 0.0 | 0.0 | 0.0 | 0.0 |
| PHE 8 B | −2.0 | −1.5 | −1.7 | −1.2 | −0.2 | 0.0 | 0.0 | 0.0 | 0.0 | 0.0 | 0.0 | 0.0 | 0.0 |
| LEU 9 B | −0.7 | −1.8 | 0.0 | −0.1 | 0.0 | 0.0 | 0.0 | 0.0 | −1.0 | −0.3 | −2.4 | −3.3 | 0.0 |
| SER 12 B | 0.2 | 0.0 | 1.0 | 0.0 | 0.0 | 0.0 | 0.0 | 0.0 | 0.0 | 0.0 | 0.0 | 0.0 | 0.0 |
| GLN 16 B | 0.9 | 0.0 | 0.0 | 0.0 | 0.0 | 0.0 | 0.0 | 0.0 | 0.0 | 0.0 | 0.0 | 0.0 | 0.0 |
| HIS 93 B | 0.0 | 0.0 | 0.0 | 0.0 | 0.0 | 0.0 | 0.0 | 0.0 | 0.0 | 0.0 | −0.8 | −2.1 | 0.9 |
| ASN 96 B | 0.0 | 0.0 | 0.0 | 0.0 | 0.0 | 0.0 | 0.0 | 0.0 | 0.0 | 0.0 | −0.4 | 0.0 | 1.0 |
| HIS 97 B | 0.0 | 0.0 | 0.0 | 0.0 | 0.0 | 0.0 | 0.0 | 0.0 | −0.8 | 0.0 | −2.4 | −2.0 | 1.9 |
| THR 100 B | 0.0 | 0.0 | 0.0 | 0.0 | 0.0 | 0.0 | 0.0 | 0.3 | −1.7 | 0.0 | −0.7 | 0.0 | 0.0 |
| VAL 101 B | 0.0 | 0.0 | 0.0 | 0.0 | 0.0 | 0.0 | 0.0 | 0.6 | −1.6 | 0.0 | 0.0 | 0.0 | 0.0 |
| GLU 104 B | 0.0 | 0.0 | 0.0 | 0.0 | 0.0 | 0.0 | 0.0 | −2.6 | −0.5 | 0.0 | 0.0 | 0.0 | 0.0 |

Residues that participate in at least one intermolecular interaction that is at least 1 kcal/mol in magnitude may play a role in dimer formation; those residues that form several favorable interactions are especially likely to be critical for dimerization.

Next, SPA calculations were used to identify suitable replacements for the dimer interface residues. Two sets of calculations were performed for each interface residue. First, the energy of the most favorable rotamer for each possible residue was determined in the context of the monomer structure (chain A or chain B, PDB code 1AU1). Next, the energy of the most favorable rotamer for each possible residue was determined in the context of the dimer structure (chains A and B, PDB code 1AU1). These energies were analyzed to identify residues that are compatible with the monomer structure but not the dimer structure. Residues were deemed compatible with the monomer structure if their energy score in the monomer structure was better than 2, and residues were deemed incompatible with the dimer structure if their energy score in the dimer structure was worse than 2.

TABLE 13
SPA energies in the context of the monomer structure.
The residue number and chain identifier are shown in the left, along with the
residue observed in wild type interferon beta. Energy scores were truncated
at 50.0.

| | | | A | C | D | E | F | G | H | I | K | L |
|---|---|---|---|---|---|---|---|---|---|---|---|---|
| 42 | A | E | 0.5 | 2.0 | 0.3 | 0.9 | 3.0 | 3.8 | 3.1 | 1.5 | 1.5 | 1.4 |
| 43 | A | E | 1.4 | 1.9 | 2.9 | 1.3 | 1.1 | 6.6 | 3.0 | 1.8 | 0.9 | 0.0 |
| 46 | A | Q | 0.9 | 1.9 | 1.7 | 0.6 | 1.8 | 4.1 | 2.2 | 11.2 | 0.4 | 1.1 |
| 47 | A | L | 3.6 | 4.0 | 4.2 | 1.7 | 20.0 | 6.8 | 5.7 | 20.0 | 1.4 | 3.9 |
| 48 | A | Q | 1.7 | 2.8 | 1.1 | 1.6 | 4.3 | 4.6 | 3.3 | 2.1 | 2.1 | 2.9 |
| 49 | A | Q | 1.0 | 2.1 | 0.5 | 0.8 | 3.4 | 3.3 | 2.8 | 3.7 | 1.9 | 2.3 |
| 51 | A | Q | 1.0 | 2.8 | 3.5 | 1.3 | 3.2 | 4.9 | 2.5 | 4.0 | 1.0 | 1.9 |
| 113 | A | R | 0.9 | 1.8 | 1.5 | 0.5 | 1.5 | 3.4 | 1.7 | 2.6 | 1.1 | 1.5 |
| 116 | A | L | 0.3 | 2.0 | 1.4 | 0.0 | 2.7 | 4.1 | 3.4 | 1.7 | 1.2 | 1.0 |
| 117 | A | M | 2.2 | 4.0 | 5.1 | 8.0 | 19.7 | 7.7 | 12.9 | 1.1 | 4.7 | 7.3 |
| 120 | A | L | 1.9 | 2.9 | 1.5 | 2.2 | 2.1 | 4.5 | 3.4 | 9.4 | 1.4 | 1.8 |
| 121 | A | H | 1.5 | 3.1 | 1.9 | 1.6 | 1.5 | 5.6 | 2.9 | 20.0 | 0.1 | 1.6 |
| 124 | A | R | 0.3 | 1.6 | 1.3 | 0.0 | 4.0 | 4.2 | 1.7 | 0.7 | 1.0 | 0.9 |
| 1 | B | M | 0.5 | 2.0 | 0.4 | 0.5 | 3.9 | 2.8 | 2.9 | 3.4 | 1.5 | 2.4 |
| 2 | B | S | 4.1 | 4.6 | 4.3 | 3.9 | 5.5 | 0.0 | 4.0 | 3.9 | 2.4 | 4.7 |
| 3 | B | Y | 5.7 | 5.8 | 7.3 | 5.8 | 2.1 | 9.2 | 5.5 | 11.9 | 4.2 | 4.2 |
| 4 | B | L | 1.9 | 2.4 | 0.5 | 0.6 | 4.5 | 5.4 | 5.2 | 1.5 | 1.9 | 2.8 |
| 5 | B | L | 0.5 | 1.8 | 0.3 | 0.0 | 2.4 | 4.4 | 2.7 | 0.7 | 1.0 | 0.6 |
| 6 | B | L | 5.4 | 7.0 | 6.4 | 5.5 | 20.0 | 10.1 | 12.3 | 20.0 | 5.5 | 0.0 |
| 7 | B | G | 50.0 | 50.0 | 50.0 | 50.0 | 50.0 | 0.0 | 50.0 | 50.0 | 50.0 | 50.0 |
| 8 | B | F | 0.8 | 1.9 | 1.2 | 0.0 | 2.4 | 4.5 | 3.0 | 5.9 | 1.5 | 0.9 |
| 9 | B | L | 2.3 | 3.5 | 4.0 | 2.5 | 7.0 | 7.6 | 3.7 | 1.4 | 0.3 | 0.0 |
| 12 | B | S | 0.3 | 1.2 | 0.3 | 0.3 | 1.8 | 4.4 | 3.4 | 0.5 | 0.8 | 0.3 |
| 16 | B | Q | 0.0 | 1.5 | 0.0 | 0.3 | 4.7 | 4.5 | 1.8 | 0.3 | 0.4 | 1.1 |
| 93 | B | H | 0.1 | 1.7 | 1.7 | 0.5 | 5.3 | 4.3 | 1.6 | 0.7 | 0.4 | 0.1 |
| 96 | B | N | 1.3 | 2.0 | 1.6 | 0.0 | 3.0 | 5.2 | 2.0 | 0.6 | 0.6 | 0.0 |
| 97 | B | H | 1.6 | 3.1 | 3.4 | 2.3 | 6.5 | 7.1 | 2.7 | 0.0 | 1.5 | 3.8 |
| 100 | B | T | 0.9 | 2.2 | 2.4 | 1.1 | 2.8 | 5.0 | 2.8 | 0.7 | 0.8 | 0.0 |
| 101 | B | V | 2.4 | 3.6 | 4.5 | 9.2 | 20.0 | 8.3 | 8.9 | 1.4 | 3.9 | 13.0 |
| 104 | B | E | 1.7 | 3.6 | 4.5 | 1.3 | 4.6 | 5.4 | 3.6 | 3.2 | 0.4 | 0.8 |
| | | | M | N | P | Q | R | S | T | V | W | Y |
| 42 | A | E | 2.3 | 0.1 | 0.0 | 0.4 | 1.3 | 0.1 | 0.5 | 2.1 | 5.4 | 2.7 |
| 43 | A | E | 1.8 | 2.5 | 2.0 | 1.2 | 0.7 | 2.2 | 1.1 | 0.6 | 3.7 | 1.5 |
| 46 | A | Q | 2.7 | 0.0 | 50.0 | 0.0 | 0.4 | 0.6 | 2.1 | 8.5 | 5.7 | 1.4 |
| 47 | A | L | 2.4 | 2.6 | 50.0 | 0.0 | 2.5 | 3.7 | 7.5 | 20.0 | 50.0 | 50.0 |
| 48 | A | Q | 2.9 | 0.0 | 3.9 | 0.9 | 2.3 | 1.2 | 1.2 | 2.9 | 7.0 | 3.7 |
| 49 | A | Q | 3.4 | 0.0 | 4.9 | 0.5 | 1.4 | 0.2 | 1.6 | 2.9 | 5.8 | 3.3 |
| 51 | A | Q | 3.3 | 1.0 | 0.0 | 0.9 | 1.3 | 0.5 | 3.2 | 3.2 | 5.6 | 3.2 |
| 113 | A | R | 2.0 | 0.0 | 50.0 | 0.3 | 0.3 | 0.2 | 1.8 | 2.2 | 5.0 | 1.4 |
| 116 | A | L | 2.8 | 0.5 | 50.0 | 0.1 | 1.5 | 0.2 | 0.7 | 1.8 | 5.4 | 3.0 |
| 117 | A | M | 3.3 | 3.7 | 5.0 | 6.9 | 1.8 | 2.9 | 1.7 | 0.0 | 20.0 | 13.8 |
| 120 | A | L | 2.8 | 0.0 | 17.7 | 2.6 | 2.6 | 2.1 | 3.9 | 8.2 | 5.9 | 1.7 |
| 121 | A | H | 2.6 | 0.0 | 20.0 | 0.9 | 0.8 | 1.9 | 1.1 | 10.2 | 4.2 | 1.8 |
| 124 | A | R | 2.1 | 1.0 | 50.0 | 0.5 | 1.3 | 0.4 | 0.9 | 0.5 | 6.5 | 4.0 |
| 1 | B | M | 3.4 | 0.1 | 3.6 | 0.2 | 0.9 | 0.0 | 1.7 | 2.6 | 6.5 | 3.7 |
| 2 | B | S | 4.4 | 2.5 | 50.0 | 3.3 | 3.4 | 3.3 | 2.1 | 6.3 | 7.8 | 6.3 |
| 3 | B | Y | 3.8 | 5.4 | 50.0 | 6.0 | 8.2 | 6.0 | 14.7 | 12.9 | 0.0 | 2.5 |
| 4 | B | L | 3.7 | 0.0 | 5.8 | 1.1 | 2.3 | 1.2 | 1.5 | 1.4 | 6.5 | 4.8 |
| 5 | B | L | 1.6 | 0.4 | 5.5 | 0.2 | 0.6 | 0.4 | 0.6 | 0.3 | 4.1 | 2.3 |
| 6 | B | L | 4.5 | 6.0 | 50.0 | 6.3 | 16.6 | 7.4 | 10.8 | 50.0 | 20.0 | 20.0 |
| 7 | B | G | 50.0 | 50.0 | 50.0 | 50.0 | 50.0 | 50.0 | 50.0 | 50.0 | 50.0 | 50.0 |
| 8 | B | F | 3.2 | 0.6 | 50.0 | 0.2 | 1.3 | 0.8 | 2.6 | 9.5 | 4.3 | 2.9 |
| 9 | B | L | 2.1 | 3.1 | 50.0 | 2.2 | 3.2 | 1.1 | 2.7 | 2.5 | 8.8 | 7.0 |
| 12 | B | S | 1.4 | 0.0 | 50.0 | 0.1 | 1.0 | 0.6 | 0.7 | 0.9 | 2.9 | 2.3 |
| 16 | B | Q | 0.9 | 0.7 | 50.0 | 0.6 | 1.9 | 0.1 | 1.3 | 0.4 | 7.5 | 4.5 |
| 93 | B | H | 1.9 | 0.9 | 50.0 | 0.0 | 1.0 | 0.4 | 0.8 | 1.3 | 8.1 | 4.7 |
| 96 | B | N | 2.0 | 1.7 | 50.0 | 0.3 | 1.3 | 1.2 | 1.7 | 1.6 | 5.9 | 3.4 |
| 97 | B | H | 2.8 | 0.1 | 50.0 | 2.6 | 2.6 | 1.8 | 2.0 | 0.0 | 8.1 | 10.4 |
| 100 | B | T | 2.4 | 1.5 | 50.0 | 0.6 | 0.8 | 1.3 | 1.6 | 1.8 | 6.5 | 3.1 |
| 101 | B | V | 7.9 | 4.0 | 50.0 | 9.9 | 6.4 | 3.5 | 2.0 | 0.0 | 20.0 | 20.0 |
| 104 | B | E | 2.1 | 2.7 | 50.0 | 0.0 | 1.4 | 0.0 | 1.0 | 4.1 | 7.8 | 4.9 |

TABLE 14
SPA energies in the context of the dimer structure.
The residue number and chain identifier are shown in the left, along with the
residue observed in wild type interferon beta. Energy scores were truncated
at 50.0.

| | | | A | C | D | E | F | G | H | I | K | L |
|---|---|---|---|---|---|---|---|---|---|---|---|---|
| 42 | A | E | 0.9 | 2.6 | 1.0 | 1.3 | 2.8 | 4.9 | 3.4 | 0.6 | 1.2 | 0.9 |
| 43 | A | E | 0.5 | 1.7 | 6.2 | 2.5 | 20.0 | 7.0 | 8.0 | 0.9 | 3.0 | 7.7 |
| 46 | A | Q | 0.7 | 1.9 | 1.9 | 0.4 | 1.0 | 4.5 | 2.0 | 20.0 | 0.0 | 0.5 |
| 47 | A | L | 4.0 | 4.3 | 4.1 | 1.7 | 14.0 | 8.3 | 3.8 | 20.0 | 1.4 | 1.9 |
| 48 | A | Q | 1.7 | 2.6 | 0.9 | 1.6 | 3.8 | 4.6 | 3.2 | 1.9 | 2.2 | 2.7 |
| 49 | A | Q | 1.4 | 2.9 | 0.8 | 2.3 | 2.5 | 4.8 | 2.9 | 3.0 | 2.5 | 3.9 |
| 51 | A | Q | 1.2 | 2.7 | 3.6 | 1.9 | 2.1 | 5.5 | 3.2 | 3.9 | 1.2 | 1.5 |
| 113 | A | R | 1.7 | 3.4 | 4.1 | 2.2 | 0.0 | 5.1 | 1.0 | 2.0 | 0.0 | 0.3 |
| 116 | A | L | 1.9 | 3.3 | 4.4 | 2.3 | 0.0 | 6.9 | 2.7 | 1.3 | 1.7 | 3.0 |
| 117 | A | M | 2.3 | 4.3 | 5.1 | 7.2 | 20.0 | 8.1 | 15.5 | 3.0 | 6.6 | 7.1 |
| 120 | A | L | 1.6 | 2.7 | 1.9 | 2.3 | 0.7 | 4.7 | 2.6 | 8.0 | 0.9 | 0.6 |
| 121 | A | H | 2.5 | 3.9 | 3.0 | 2.3 | 3.0 | 6.7 | 3.4 | 20.0 | 0.3 | 1.9 |
| 124 | A | R | 0.4 | 1.6 | 1.4 | 0.0 | 3.8 | 4.3 | 1.9 | 0.9 | 1.2 | 0.9 |
| 1 | B | M | 0.4 | 1.9 | 0.7 | 1.2 | 2.1 | 3.3 | 3.1 | 3.1 | 0.5 | 1.7 |
| 2 | B | S | 2.9 | 3.0 | 5.9 | 9.3 | 12.8 | 0.0 | 5.7 | 6.0 | 5.8 | 20.0 |
| 3 | B | Y | 5.9 | 6.0 | 6.4 | 5.5 | 2.3 | 9.4 | 5.6 | 12.2 | 5.2 | 4.4 |
| 4 | B | N | 2.4 | 2.9 | 0.2 | 1.6 | 8.6 | 6.9 | 6.9 | 2.0 | 2.1 | 2.0 |
| 5 | B | L | 4.0 | 5.7 | 5.2 | 6.7 | 3.4 | 9.8 | 3.8 | 0.0 | 5.3 | 6.9 |
| 6 | B | L | 5.4 | 7.0 | 6.5 | 4.9 | 20.0 | 10.1 | 14.0 | 20.0 | 5.9 | 0.0 |
| 7 | B | G | 50.0 | 50.0 | 50.0 | 50.0 | 50.0 | 0.0 | 50.0 | 50.0 | 50.0 | 50.0 |
| 8 | B | F | 4.9 | 6.0 | 7.3 | 4.4 | 0.0 | 9.8 | 4.7 | 17.5 | 4.1 | 5.2 |
| 9 | B | L | 2.9 | 4.7 | 5.9 | 4.2 | 2.8 | 8.5 | 2.6 | 1.9 | 0.0 | 0.1 |
| 12 | B | S | 0.1 | 1.5 | 0.7 | 7.3 | 9.1 | 4.9 | 16.5 | 2.0 | 5.9 | 6.0 |
| 16 | B | Q | 0.1 | 1.6 | 0.3 | 0.7 | 4.7 | 4.6 | 2.0 | 0.3 | 0.0 | 1.1 |
| 93 | B | H | 0.0 | 1.7 | 1.1 | 0.0 | 5.4 | 4.3 | 1.6 | 0.6 | 0.7 | 0.0 |
| 96 | B | N | 1.4 | 2.0 | 1.6 | 0.1 | 3.1 | 5.3 | 1.8 | 0.8 | 1.0 | 0.0 |
| 97 | B | H | 1.9 | 3.4 | 3.4 | 2.7 | 5.3 | 7.6 | 2.8 | 0.0 | 1.5 | 3.4 |
| 100 | B | T | 1.1 | 2.6 | 2.3 | 1.3 | 1.8 | 5.5 | 2.6 | 0.7 | 1.3 | 0.0 |
| 101 | B | V | 2.0 | 2.6 | 3.1 | 9.0 | 20.0 | 7.9 | 15.0 | 18.3 | 6.5 | 20.0 |
| 104 | B | E | 2.0 | 3.4 | 4.3 | 2.6 | 2.8 | 6.4 | 5.6 | 3.2 | 0.0 | 7.9 |
| | | | M | N | P | Q | R | S | T | V | W | Y |
| 42 | A | E | 2.6 | 0.8 | 0.0 | 0.2 | 2.2 | 1.0 | 1.0 | 1.8 | 5.5 | 2.8 |
| 43 | A | E | 2.6 | 5.7 | 0.2 | 2.7 | 11.8 | 2.1 | 0.9 | 0.0 | 20.0 | 20.0 |
| 46 | A | Q | 2.4 | 0.3 | 50.0 | 0.1 | 0.3 | 0.5 | 4.8 | 20.0 | 5.0 | 0.8 |
| 47 | A | L | 1.3 | 2.6 | 50.0 | 0.0 | 3.7 | 4.8 | 8.0 | 20.0 | 50.0 | 50.0 |
| 48 | A | Q | 2.8 | 0.0 | 4.0 | 0.9 | 2.0 | 1.0 | 1.0 | 2.9 | 6.0 | 3.4 |
| 49 | A | Q | 3.6 | 0.0 | 4.3 | 2.4 | 1.9 | 1.6 | 2.3 | 2.3 | 4.3 | 2.6 |
| 51 | A | Q | 2.8 | 2.1 | 0.0 | 1.6 | 1.7 | 0.7 | 3.6 | 3.4 | 2.0 | 1.7 |
| 113 | A | R | 2.6 | 0.8 | 50.0 | 1.7 | 0.3 | 1.7 | 2.3 | 2.0 | 2.7 | 0.3 |
| 116 | A | L | 2.0 | 3.7 | 50.0 | 2.9 | 5.1 | 1.3 | 0.9 | 1.6 | 20.0 | 1.8 |
| 117 | A | M | 3.3 | 4.0 | 4.9 | 6.9 | 4.8 | 3.1 | 1.5 | 0.0 | 20.0 | 20.0 |
| 120 | A | L | 1.7 | 0.0 | 19.0 | 2.9 | 3.4 | 2.0 | 2.1 | 7.0 | 3.4 | 0.3 |
| 121 | A | H | 2.4 | 0.0 | 20.0 | 2.3 | 2.1 | 2.5 | 1.1 | 10.6 | 12.3 | 8.9 |
| 124 | A | R | 2.1 | 1.2 | 50.0 | 0.7 | 1.4 | 0.3 | 0.9 | 0.5 | 6.3 | 4.3 |
| 1 | B | M | 3.0 | 0.1 | 2.9 | 1.0 | 0.5 | 0.0 | 1.4 | 1.7 | 5.8 | 4.2 |
| 2 | B | S | 6.4 | 4.2 | 50.0 | 17.7 | 11.0 | 2.3 | 1.5 | 4.2 | 20.0 | 8.9 |
| 3 | B | Y | 4.0 | 7.2 | 50.0 | 6.3 | 9.3 | 6.5 | 15.3 | 12.6 | 0.0 | 2.2 |
| 4 | B | N | 3.0 | 0.0 | 6.1 | 1.9 | 3.7 | 2.2 | 2.4 | 2.7 | 50.0 | 9.3 |
| 5 | B | L | 4.0 | 4.6 | 8.4 | 8.4 | 10.1 | 5.0 | 3.5 | 1.1 | 20.0 | 4.4 |
| 6 | B | L | 4.4 | 6.1 | 50.0 | 6.3 | 17.9 | 7.3 | 11.0 | 50.0 | 20.0 | 20.0 |
| 7 | B | G | 50.0 | 50.0 | 50.0 | 50.0 | 50.0 | 50.0 | 50.0 | 50.0 | 50.0 | 50.0 |
| 8 | B | F | 4.9 | 5.9 | 50.0 | 3.7 | 8.0 | 6.1 | 5.7 | 13.8 | 10.2 | 5.6 |
| 9 | B | L | 2.1 | 4.9 | 50.0 | 3.6 | 4.3 | 1.6 | 3.8 | 3.2 | 20.0 | 3.1 |
| 12 | B | S | 4.8 | 0.4 | 50.0 | 7.4 | 7.6 | 0.9 | 1.2 | 0.0 | 9.8 | 8.4 |
| 16 | B | Q | 1.2 | 0.9 | 50.0 | 0.6 | 0.5 | 0.1 | 1.2 | 0.3 | 6.0 | 4.7 |
| 93 | B | H | 1.5 | 1.0 | 50.0 | 0.1 | 1.6 | 0.7 | 0.9 | 1.1 | 8.9 | 4.6 |
| 96 | B | N | 2.1 | 2.0 | 50.0 | 0.5 | 2.1 | 1.2 | 1.8 | 1.6 | 5.7 | 3.5 |
| 97 | B | H | 2.1 | 0.8 | 50.0 | 2.9 | 3.8 | 2.5 | 2.3 | 0.5 | 20.0 | 20.0 |
| 100 | B | T | 2.5 | 2.1 | 50.0 | 1.1 | 1.6 | 1.9 | 1.9 | 1.9 | 6.1 | 2.5 |
| 101 | B | V | 12.3 | 3.3 | 50.0 | 10.5 | 10.3 | 3.4 | 1.5 | 0.0 | 20.0 | 20.0 |
| 104 | B | E | 2.5 | 4.3 | 50.0 | 1.6 | 3.0 | 0.1 | 0.6 | 3.6 | 3.6 | 4.2 |

TABLE 15
Suitable replacements for dimer interface positions, as determined by the above SPA calculations.

| | | | A | C | D | E | F | G | H | I | K | L | M | N | P | Q | R | S | T | V | W | Y |
|---|---|---|---|---|---|---|---|---|---|---|---|---|---|---|---|---|---|---|---|---|---|---|
| 42 | A | E | | | | | | | | | | | | | | | | | | | | |
| 43 | A | E | | | | | F | | | | K | L | | | | | R | | | | | Y |
| 46 | A | Q | | | | | | | | | | | | | | | | | | | | |
| 47 | A | L | | | | | | | | | | | | | | | | | | | | |
| 48 | A | Q | | | | | | | | | | | | | | | | | | | | |
| 49 | A | Q | | | | | | | | | | | | | | | | | | | | |
| 51 | A | Q | | | | | | | | | | | | | | | | | | | | |
| 113 | A | R | | | D | | | | | | | | | | | | | | | | | |
| 116 | A | L | | | D | E | | | | | | L | | N | | Q | R | | | | | |
| 117 | A | M | | | | | | | | | | | | | | | R | | | | | |
| 120 | A | L | | | | | | | | | | | | | | | | | | | | |
| 121 | A | H | | | | | | | | | | | | | | | | | | | | Y |
| 124 | A | R | | | | | | | | | | | | | | | | | | | | |
| 1 | B | M | | | | | | | | | | | | | | | | | | | | |
| 2 | B | S | | | | | | | | | | | | | | | | | | | | |
| 3 | B | Y | | | | | | | | | | | | | | | | | | | | |
| 4 | B | L | | | | | | | | | | | | | | | | | | | | |
| 5 | B | L | A | C | D | E | | | | | K | L | M | N | | Q | R | S | T | | | |
| 6 | B | L | | | | | | | | | | | | | | | | | | | | |
| 7 | B | G | | | | | | | | | | | | | | | | | | | | |
| 8 | B | F | A | C | D | E | | | | | K | L | | N | | Q | R | S | | | | |
| 9 | B | L | | | | | | | | | | | | | | | | | | | | |
| 12 | B | S | | | | E | F | | | | K | L | M | | | Q | R | | | | | |
| 16 | B | Q | | | | | | | | | | | | | | | | | | | | |
| 93 | B | H | | | | | | | | | | | | | | | | | | | | |
| 96 | B | N | | | | | | | | | | | | | | | | | | | | |
| 97 | B | H | | | | | | | | | | | | | | | | | | | | |
| 100 | B | T | | | | | | | | | | | | | | | | | | | | |
| 101 | B | V | | | | | | | | I | | | | | | | | | | | | |
| 104 | B | E | | | | | | | | | | L | | | | | | | | | | |

As can be observed in the tables above, positions 5, 8, 12, 43, and 116 are all involved in stabilizing the dimer structure of interferon-beta, and a number of modifications at these positions are predicted to significantly prevent dimerization.

Further analysis was performed to determine which of the above modifications is most likely to significantly prevent dimerization. Hydrophobic interactions and electrostatic interactions (including salt bridges and hydrogen bonds) can stabilize protein-protein interfaces. These interactions may be effectively disrupted by hydrophobic to polar and charge reversal mutations.

Hydrophobic residues that are significantly less solvent exposed in the dimer structure versus the monomer structure were defined to be those residues that are classified as surface in the monomer and core or boundary in the dimer, and residues that are classified as boundary in the monomer and core in the dimer, as shown below:

TABLE 16
Hydrophobic residues that are more buried
in the dimer than in the monomer.

| Residue | Monomer | Dimer |
|---|---|---|
| Leu 5 | Boundary | Core |
| Phe 8 | Surface | Core |
| Leu 9 | Boundary | Core |
| Leu 47 | Boundary | Core |
| Leu 116 | Surface | Boundary |

Debye-Huckel scaled Coulomb's law calculations were performed on the 1AU1 dimer and monomers, using an ionic strength of 0.15 M, to determine the electrostatic potential at each position in the context of the monomer versus the dimer. The following positions were found to have a change in potential of at least 0.20 kcal/mol:

TABLE 17
Positions that experience a significant difference in electrostatic
potential in the dimer versus monomer structure.

| | | | Dimer | Monomer | Difference |
|---|---|---|---|---|---|
| SER | 2 | B | 0.36 | −0.30 | 0.66 |
| LEU | 5 | B | −0.10 | 0.11 | −0.21 |
| PHE | 8 | B | 0.14 | 0.42 | −0.28 |
| LEU | 9 | B | −0.11 | 0.16 | −0.27 |
| SER | 12 | B | −0.42 | 0.29 | −0.71 |
| LEU | 47 | A | 0.25 | 0.04 | 0.21 |
| GLN | 49 | A | 0.32 | 0.08 | 0.24 |
| HIS | 93 | B | 0.29 | 0.04 | 0.25 |
| ASN | 96 | B | 0.24 | 0.04 | 0.20 |
| THR | 100 | B | −0.22 | −0.45 | 0.23 |
| VAL | 101 | B | 0.15 | −0.39 | 0.54 |
| GLU | 104 | B | 0.58 | −0.02 | 0.60 |
| ARG | 113 | A | −1.37 | −0.36 | −1.01 |

Modifications of the electrostatic properties of the residues at these positions can be selected to favor the monomer structure and disfavor the dimer structure. For example, Glu 104 and Arg 113 form a salt bridge in the dimer structure, which can be observed in the crystal structure. In the table above, Glu 104 is in a region of positive potential in the dimer and neutral potential in the monomer, while Arg 113 is in a region of negative potential in the dimer structure and slightly negative potential in the monomer structure. Modifications that could disrupt this interaction include, but are not limited to, E104R, E104K, E104H, E104Q, E104A, R113D, R113E, R113Q, and R113A.

### Example 7

### Identification of Suitable Replacements for Free Cysteine Residues

PDA® technology calculations were also performed to identify suitable replacements for free cysteine residues. These calculations were performed using the methods described above for the hydrophobic to polar point mutations, except that both polar and nonpolar replacements were considered. Alternate residues with favorable energies are marked with a star (*) below.

TABLE 18
Free cysteine calculation results

| | AA | Total | VDW | Elec | HBond | Solv |
|---|---|---|---|---|---|---|
| | IFNa | | | | | |
| | TYR-C | −13.47 | −10.45 | −0.11 | −2.32 | −0.59 |
| | ILE | 15.37 | 13.90 | 0.00 | 0.00 | 1.47 |
| * | LEU | −5.58 | −5.38 | 0.00 | 0.00 | −0.20 |
| * | MET | −6.17 | −5.42 | 0.00 | 0.00 | −0.75 |
| | PHE | 887.53 | 893.12 | 0.00 | 0.00 | −5.59 |
| | TRP | 0.98 | −6.86 | −0.01 | 0.00 | 7.86 |
| | TYR | 803.08 | 804.33 | −0.02 | 0.00 | −1.23 |
| | VAL | 27.93 | 29.08 | 0.00 | 0.00 | −1.15 |
| | ALA | −2.53 | −1.89 | 0.00 | 0.00 | −0.63 |
| * | ASP | −4.45 | −4.05 | 0.33 | 0.00 | −0.73 |
| * | GLU | −7.53 | −4.66 | 0.39 | 0.00 | −3.26 |
| * | HIS | −5.94 | −6.12 | −0.12 | 0.00 | 0.30 |
| * | HSP | −4.19 | −5.94 | −0.76 | 0.00 | 2.51 |
| * | LYS | −8.48 | −5.48 | −0.38 | 0.00 | −2.63 |
| | ASN | −3.00 | −4.12 | −0.03 | 0.00 | 1.15 |
| * | GLN | −8.21 | −4.70 | −0.01 | 0.00 | −3.50 |
| * | ARG | −4.73 | −5.42 | −0.24 | 0.00 | 0.93 |
| | SER | −4.04 | −2.17 | −0.02 | 0.00 | −1.85 |
| * | THR | −5.10 | −3.08 | −0.02 | 0.00 | −2.01 |
| | IFNb | | | | | |
| | CYS-C | −13.97 | −7.06 | 0.00 | 0.00 | −6.91 |
| | ILE | 324.91 | 334.90 | 0.00 | 0.00 | −9.99 |
| | LEU | 840.30 | 846.29 | 0.00 | 0.00 | −5.99 |
| | MET | 2082.91 | 2089.08 | 0.00 | 0.00 | −6.17 |
| | PHE | 5529.90 | 5539.67 | 0.00 | 0.00 | −9.77 |
| | TYR | 6341.29 | 6346.98 | −0.26 | 0.00 | −5.43 |
| | VAL | 82.62 | 89.33 | 0.00 | 0.00 | −6.70 |
| * | ALA | −8.69 | −3.42 | 0.00 | 0.00 | −5.27 |
| * | ASP | −10.20 | −7.37 | 0.12 | 0.00 | −2.96 |
| | GLU | 357.99 | 358.18 | 0.42 | 0.00 | −0.62 |
| | HIS | 501.55 | 504.61 | −0.05 | 0.00 | −3.01 |
| | HSP | 506.45 | 506.93 | 0.35 | 0.00 | −0.83 |
| | LYS | 2087.79 | 2085.18 | −0.04 | 0.00 | 2.64 |
| * | ASN | −5.08 | −6.54 | 0.11 | 0.00 | 1.36 |
| | GLN | 483.14 | 479.27 | 0.10 | 0.00 | 3.77 |
| | ARG | 15093.59 | 15085.56 | 0.04 | 0.00 | 7.99 |
| * | SER | −5.96 | −4.41 | −0.08 | 0.00 | −1.47 |
| * | THR | −9.17 | −5.20 | 0.06 | 0.00 | −4.03 |
| | IFNk | | | | | |
| | LEU-C | 5507.86 | 5514.27 | −0.41 | 0.00 | −6.01 |
| | ILE | 44.93 | 50.89 | 0.00 | 0.00 | −5.96 |
| | LEU | −13.20 | −7.12 | 0.00 | 0.00 | −6.08 |
| * | MET | −3.21 | 3.30 | 0.00 | 0.00 | −6.51 |
| | PHE | 36.05 | 43.81 | 0.00 | 0.00 | −7.76 |
| | TRP | 292.31 | 298.19 | −0.01 | 0.00 | −5.87 |
| | TYR | 196.77 | 200.15 | −0.01 | 0.00 | −3.37 |
| | VAL | 37.53 | 42.27 | 0.00 | 0.00 | −4.74 |
| * | ALA | −7.83 | −2.63 | 0.00 | 0.00 | −5.20 |
| | ASP | −4.81 | −5.70 | −0.12 | 0.00 | 1.01 |
| * | GLU | −9.02 | −8.02 | −0.17 | 0.00 | −0.83 |
| * | HIS | −10.31 | −9.00 | −0.11 | 0.00 | −1.21 |
| * | HSP | −7.47 | −8.25 | −0.23 | 0.00 | 1.00 |
| | LYS | 2.43 | 0.20 | 0.02 | 0.00 | 2.22 |
| | ASN | −0.48 | −5.83 | 0.00 | 0.00 | 5.35 |
| * | GLN | −4.21 | −7.92 | −0.03 | 0.00 | 3.74 |
| | ARG | 52.67 | 44.39 | 0.01 | 0.00 | 8.27 |
| * | SER | −4.86 | −3.32 | 0.00 | 0.00 | −1.54 |
| * | THR | −3.56 | −3.63 | −0.10 | 0.00 | 0.18 |

### Example 8

### Generation of Interferon Beta Variants

Construction of the interferon beta gene as a template for mutagenesis. The DNA sequence, GenBank accession number NM_{—}002176, encompassing the full-length human interferon beta cDNA gene containing the native signal sequence was modified to remove the signal sequence and facilitate high level expression in bacterial cells. Primers were designed to synthesize the region between positions 65-561 by recursive PCR. The primer sequences also biased the codon usage towards highly expressed E. coli bacterial genes. In addition, the codon for cysteine 17 (amino acid numbering with the signal sequence removed) was changed to serine. An internal Sac DNA restriction enzyme site was designed for ease of later mutagenesis as well as NheI and XhoI restriction sites flanking the ends of the gene for cassette cloning into various expression vectors pET28a and pET24a (Novagen) were used to sub-clone the interferon beta gene containing C17S between the NdeI and XhoI multiple cloning restriction sites. Cloning into pET24a expression in E. coli produces a C17S interferon beta variant while cloning into pET28a introduces the additional amino acid sequence MGSSHHHHHHSSGLVPRGSH to the N-terminus of C17S. This amino acid sequence includes a 6-His purification tag and a thrombin cleavage site for later removal of the added amino acid sequences.

Construction of interferon beta variants containing exposed hydrophobic to polar mutations

Sixteen solvent exposed hydrophobic residues were identified in the interferon beta structure. Polar amino acid residues to substitute at these positions were designed by computational analysis as described above. The list of substitutions are listed in the table below:

TABLE 19
List of substitutions used in library
of interferon beta variants

| position | wt | LIB |
|---|---|---|
| 5 | L | Q |
| 8 | F | E |
| 15 | F | D |
| 22 | W | E |
| 28 | L | Q |
| 30 | Y | N |
| 32 | L | E |
| 36 | M | K |
| 47 | L | K |
| 92 | Y | D |
| 111 | F | N |
| 116 | L | E |
| 120 | L | R |
| 130 | L | T |
| 148 | V | E |
| 155 | Y | S |

Mutagenesis experiments were done to construct variants containing these amino acid substitutions in the interferon beta-C17S gene background (referred to as “wild type” throughout the following examples).

For a library containing combinations of the wild-type or substitution listed in the table above, a template directed ligation-PCR method was used as described in Strizhov et. al. PNAS 93:15012-15017 (1996). Variants constructed contain single or multiple combinations of the substitutions.

For a 64-member library containing all possible combinations of wild-type or above-listed substitution at positions 5,8,47,111,116, and/or 120, multiple rounds of site-directed mutagenesis reactions were done using the Quikchange kit (commercially available from Stratagene) following the manufacturer's protocol. Positive clones were identified by sequencing.

Production of interferon beta variants in E. Coli. Sequence verified clones in pET28a were transformed into BL21(DE3) star cells (commercially available from Invitrogen) and cultures were grown in auto-inducing media, a rich medium for growth with little or no induction during log phase and auto-induction of expression as the culture approaches saturation. Media components include 25 mM (NH₄)₂SO₄, 50 mM KH₂PO₄, 50 mM Na₂HPO₄, 1 mM MgSO₄, 0.5% glycerol, 0.05% glucose, 0.2% alpha-lactose, 0.1% tryptone, and 0.05% yeast extract. The cultures were grown for 7 hours to an OD between 4 and 5 and cells harvested by centrifugation. Cells were lysed by sonication, inclusion pellets denatured in 8M guanidine HCI and bound to a column containing Ni-NTA resin. A dilution series of guanidine HCI with decreasing pH was used to purify and refold the protein.

An alternative method for purification of clones with and without the N-terminal 6-His tag was followed as disclosed in U.S. Pat. No. 4,462,940, Lin et al, Meth. Enzymol. 119:183-192.

### Example 9

### Soluble Expression of Interferon Beta Variants

Each of the 64 members of the library described above were tested for soluble expression. Western blot analysis utilizing an anti-His antibody was done for the soluble fractions of cell lysates. A band running at the expected size of approximately 20 kilodaltons was present for at least 33 of the variants but was not detectable for the C17S variant, suggesting that many of the designed variants exhibit improved soluble expression.

### Example 10

### Activity Analysis of Constructed Variants

A standard ISRE (interferon-stimulated response element) reporter assay was used to determine the activity of interferon beta variants. In this assay, 293T cells which constitutively express the type I interferon receptor were transiently transfected with an ISRE-luciferase vector (pISRE-luc, commercially available from Clontech). Twelve hours after transfection, the cells were treated with a dilution series of concentrations for an interferon beta variant. Variants which bind the interferon receptor and trigger the JAK/STAT signal transduction cascade activate transcription of the luciferase gene operably linked to the ISRE. Luciferase activity was detected using the Steady-Glo® Luciferase Assay System (commercially available from Promega) with the TopCount NX™ microplate reader used to measure luminescence.

Initial activity determination utilizing the ISRE reporter assay was done for the 64 member library described in example 8. Cultures were grown, cells harvested and lysed. The inclusion pellet was resuspended in a 0.025% SDS solution and tested in the ISRE activity assay. Activity was demonstrated for the 37 variants listed in the table below. However, since the amount of protein tested in this assay was not quantitated first, it is possible that additional variants are active but were present in insufficient quantity to be detected in the assay.

TABLE 20
Amino acid sequences at exposed hydrophobic positions
for active interferon beta variants

Amino acid position

| Variant | 5 | 8 | 47 | 111 | 116 | 120 |
|---|---|---|---|---|---|---|
| IFB1_2 | Q | F | L | F | L | L |
| IFB1_3 | Q | F | K | F | L | L |
| IFB1_4 | L | E | L | F | L | L |
| IFB1_5 | L | E | K | F | L | L |
| IFB1_6 | L | F | K | F | L | L |
| IFB1_7 | Q | E | L | F | L | L |
| IFB1_8 | Q | E | K | F | L | L |
| IFB1_9 | L | F | L | N | L | L |
| IFB1_10 | Q | F | L | N | L | L |
| IFB1_11 | Q | F | K | N | L | L |
| IFB1_15 | Q | E | L | N | L | L |
| IFB1_16 | Q | E | K | N | L | L |
| IFB1_23 | Q | E | L | F | E | L |
| IFB1_26 | Q | F | L | F | L | R |
| IFB1_27 | Q | F | K | F | L | R |
| IFB1_28 | L | E | L | F | L | R |
| IFB1_29 | L | E | K | F | L | R |
| IFB1_31 | Q | E | L | F | L | R |
| IFB1_32 | Q | E | K | F | L | R |
| IFB1_33 | L | F | L | N | E | L |
| IFB1_34 | Q | F | L | N | E | L |
| IFB1_35 | Q | F | K | N | E | L |
| IFB1_36 | L | E | L | N | E | L |
| IFB1_37 | L | E | K | N | E | L |
| IFB1_39 | Q | E | L | N | E | L |
| IFB1_40 | Q | E | K | N | E | L |
| IFB1_41 | L | F | L | N | L | R |
| IFB1_42 | Q | F | L | N | L | R |
| IFB1_44 | L | E | L | N | L | R |
| IFB1_47 | Q | E | L | N | L | R |
| IFB1_48 | Q | E | K | N | L | R |
| IFB1_50 | Q | F | L | F | E | R |
| IFB1_51 | Q | F | K | F | E | R |
| IFB1_52 | L | E | L | F | E | R |
| IFB1_55 | Q | E | L | F | E | R |
| IFB1_56 | Q | E | K | F | E | R |
| IFB1_63 | Q | E | L | N | E | R |
| IFB1_64 | Q | E | K | N | E | R |

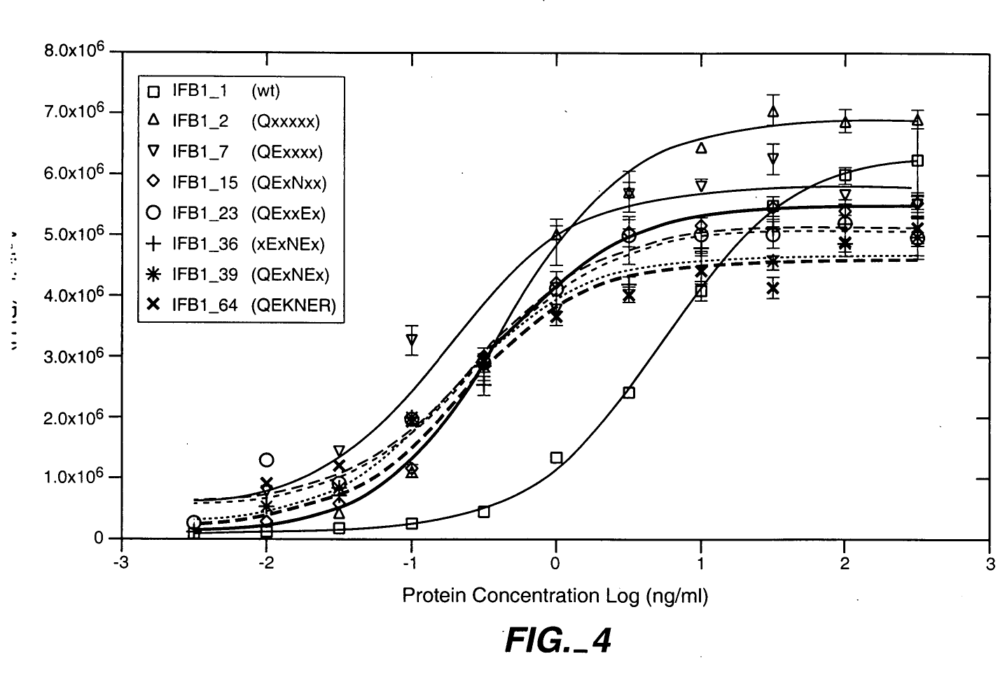

Those variants exhibiting increased activity relative to the wild type (interferon beta C17S) were tested for more quantitative activity measurements. Selected variants were purified and refolded as described in example 8 above. Each variant was then assayed using a ten point half-log dilution series in the ISRE reporter assay. GraphPad Prism®, version 4 (GraphPad Software, Inc.) was used to plot the data and calculate EC50 values. The dose response curves for the retested variants are shown in FIG. 4. All of the variants exhibited improved activity, with EC50 values ranging from 12-30 fold better activity than C17S interferon beta, as shown in the table below.

TABLE 21
Specific activity data for interferon-beta variants.

| | | | | | | | # | EC50 | EC50 |
|---|---|---|---|---|---|---|---|---|---|
| Variant | 5 | 8 | 47 | 111 | 116 | 120 | mut | (log ng/ml) | wt/EC50 var |
| IFN1_1 | L | F | L | F | L | L | 0 | 5.306 | 1.0 |
| IFB1_2 | Q | F | L | F | L | L | 1 | 0.428 | 12.4 |
| IFB1_7 | Q | E | L | F | L | L | 2 | 0.179 | 29.6 |
| IFB1_15 | Q | E | L | N | L | L | 3 | 0.319 | 16.6 |
| IFB1_23 | Q | E | L | F | E | L | 3 | 0.277 | 19.2 |
| IFB1_36 | L | E | L | N | E | L | 3 | 0.294 | 18.0 |
| IFB1_39 | Q | E | L | N | E | L | 4 | 0.193 | 27.5 |
| IFB1_64 | Q | E | K | N | E | R | 6 | 0.240 | 22.1 |

The sequence for residues 5, 8, 47, 111, 116, and 120 is given for each variant, along with the total number of mutations, the EC50, and the ratio of the wild type to variant EC50. Variant IFN1_1 is the interferon beta wild type with C17S.

Activity Comparison with claimed solubility mutant from U.S. Pat. No. 6,572,853.

Several variants with enhanced solubility were claimed in U.S. Pat. No. 6,572,853. Activity comparison of one of these claimed variants with the C17S wild type and the most active variant tested above was done. Purification of all the variants and activity evaluation was done under the same conditions with the results shown in the table below. The claimed solubility variant (IFB_GM2) exhibited 67 fold less activity than the wild type C17S interferon beta. In comparison, variant IFB1_{—}7 still exhibited better than 25 fold better activity than the wild type.

TABLE 22
Specific activity data for interferon-beta variants.

| | | | | | | | | | # | EC50 | EC50 |
|---|---|---|---|---|---|---|---|---|---|---|---|
| Variant | 5 | 8 | 47 | 50 | 106 | 111 | 116 | 120 | mut | (ng/ml) | wt/EC50 var |
| IFN1_1 | L | F | L | F | L | F | L | L | 0 | 1.90 | 1.00 |
| IFB1_7 | Q | E | L | F | L | F | L | L | 2 | 0.074 | 25.7 |
| IFB_GM2 | L | F | S | S | S | S | S | S | 6 | 130 | 0.015 |

The sequence for residues 5, 8, 47, 50, 106, 111, 116, and 120 is given for each variant, along with the total number of mutations, the EC50, and the ratio of the wild type to variant EC50. All variants are in the C17S background.

### Example 11

### Mutagenesis, Expression, and Soluble Expression Screening of Interferon Kappa

Construction of interferon kappa variants

Interferon kappa variants (total library size=1024) with the mutations listed in the table below (single and all possible multiple combinations) were constructed essentially as described above for the Interferon beta variants.

TABLE 23
List of substitutions used in library of interferon-kappa variants.
Each position or set of positions could have either the wild type
hydrophobic residue(s) or the alternate polar residue(s) listed in
the “LIB” column.

| position(s) | wt | LIB |
|---|---|---|
| 5-8 | L-V | Q-N |
| 15 | W | R |
| 28-30 | F-V | Q-R |
| 37 | I | N |
| 48-52 | Y-M | Q-N |
| 76-78 | F-Y | S-A |
| 89 | I | T |
| 97 | Y | D |
| 161 | V | A |
| 166-168-171 | C-Y—Y | A-S-T |

Expression and screening for soluble expression via dot-blot using anti-His antibodies for detection.

The soluble fraction of E. coli lysates expressing individual interferon-kappa variants were dot -blotted on nitrocellulose membranes, and the presence of soluble His-tagged protein was detected using anti-His antibodies conjugated to HRP. FIG. 5 shows the results of a dot-blot analysis. The positive clones expressing soluble interferon-kappa were regrown, and expressed protein was retested to confirm soluble expression. FIG. 6 shows a retest plate.

The soluble extract from interferon-kappa variants testing positive during the secondary screen were then analyzed by SDS-PAGE/Western blotting to confirm the presence of the correctly sized protein band. FIG. 7 is an example of these SDS-PAGE/western blot experiments, identifying several interferon-kappa variants expressing the correctly sized protein with solubility characteristics better than WT interferon-kappa. The arrow indicates the expected position of interferon-kappa protein. Lanes 2 and 3 are total soluble fraction from WT interferon-kappa expressing cells, respectively. Lanes 4-15 are soluble fractions from the lysates of different variants.

TABLE 24
Sequence analysis of selected interferon kappa
variants with improved soluble expression.

| | | | | | | | | | | |
|---|---|---|---|---|---|---|---|---|---|---|
| WT Seq | L-V | W | F-V | I | Y-M | F-Y | I | Y | V | C-Y-Y |
| Mutation | Q-N | R | Q-R | N | Q-N | S-A | T | D | A | A-S-T |
| Mutant | 5, 8 | 15 | 28, 30 | 37 | 48, 52 | 76, 78 | 89 | 97 | 161 | 166, 168, 171 |
| IK_4-G7 | L-N | R | F-V | I | Q-N | S-A | T | Y | V | C-Y-Y |
| IK_12-E4 | L-N | R | F-V | I | Q-N | S-A | T | Y | V | C-Y-Y |
| IK_2-C11 | L-N | R | Q-R | N | Y-M | S-A | T | D | A | A-S-T |
| IK_10-D8 | L-N | W | F-V | I | Q-N | F-Y | T | D | V | A-S-T |
| IK_10-H7 | L-N | W | F-V | I | Q-N | S-A | T | D | A | A-S-T |
| IK_20-B12 | L-N | W | Q-R | I | Q-N | S-A | T | Y | V | A-S-T |
| IK_3-A11 | L-N | W | Q-R | I | Y-M | S-A | T | D | A | A-S-T |
| IK_3-H7 | L-N | W | Q-R | I | Y-M | S-A | T | D | A | A-S-T |
| IK_2-F11 | L-N | W | Q-R | N | Q-N | S-A | T | Y | V | A-S-T |
| IK_3-D10 | L-V | R | F-V | I | Q-N | S-A | T | D | V | A-S-T |
| IK_3-C10 | L-V | R | F-V | I | Q-N | S-A | T | D | V | C-Y-Y |
| IK_3-H11 | L-V | R | F-V | I | Q-N | S-A | T | D | V | C-Y-Y |
| IK_21-E1 | L-V | R | F-V | I | Y-M | S-A | I | D | V | A-S-T |
| IK_4-H11 | L-V | R | F-V | I | Y-M | S-A | T | D | A | C-Y-Y |
| IK_3-A2 | L-V | R | F-V | I | Y-M | S-A | T | D | V | A-S-T |
| IK_10-D2 | L-V | R | F-V | N | Y-M | S-A | T | D | V | C-Y-Y |
| IK_12-H4 | L-V | W | F-V | I | Q-N | S-A | I | Y | V | C-Y-Y |
| IK_27-A6 | L-V | W | F-V | I | Q-N | S-A | T | D | A | C-Y-Y |
| IK_2-B4 | L-V | W | F-V | I | Q-N | S-A | T | D | V | C-Y-Y |
| IK_3-F11 | L-V | W | F-V | I | Q-N | S-A | T | D | V | C-Y-Y |
| IK_14-A9 | L-V | W | F-V | I | Y-M | F-Y | T | Y | V | C-Y-Y |
| IK_19-A5 | L-V | W | F-V | I | Y-M | S-A | I | D | A | C-Y-Y |
| IK_3-G10 | L-V | W | F-V | I | Y-M | S-A | I | D | V | C-Y-Y |
| IK_4-A2 | L-V | W | F-V | I | Y-M | S-A | I | D | V | C-Y-Y |
| IK_4-A10 | L-V | W | F-V | I | Y-M | S-A | I | D | V | C-Y-Y |
| IK_16-G2 | L-V | W | F-V | I | Y-M | S-A | T | D | A | C-Y-Y |
| IK_22-A4 | L-V | W | F-V | I | Y-M | S-A | T | D | V | A-S-T |
| IK_1-C8 | L-V | W | F-V | N | Q-N | S-A | I | D | V | C-Y-Y |
| IK_23-C10 | L-V | W | F-V | N | Q-N | S-A | I | D | V | C-Y-Y |
| IK_12-H11 | L-V | W | F-V | N | Q-N | S-A | T | Y | V | C-Y-Y |
| IK_9-H4 | L-V | W | Q-R | N | Y-M | S-A | I | D | V | A-S-T |

Variants with improved soluble expression were tested for activity using the ISRE assay, essentially as in the initial activity assay described above. A number of variants that retain interferon activity were identified, including those listed below.

TABLE 25
Sequence analysis of some of the Interferon-kappa variant, which still retain
activity, as tested in an ISRE assay as described above for interferon beta.

| | | | | | | | | | | |
|---|---|---|---|---|---|---|---|---|---|---|
| WT seq | L-V | W | F-V | I | Y-M | F-Y | I | Y | V | C-Y-Y |
| Mutations | Q-N | R | Q-R | N | Q-N | S-A | T | D | A | A-S-T |
| Variant | 5, 8 | 15 | 28, 30 | 37 | 48, 52 | 76, 78 | 89 | 97 | 161 | 166, 168, 171 |
| IK1_4_G7 | L-N | R | F-V | I | Q-N | S-A | T | Y | V | C-Y-Y |
| IK1_46_E2 | L-V | R | F-V | N | Q-N | S-A | T | D | A | A-S-T |
| IK1_47_C4 | L-V | R | F-V | I | Y-M | S-A | I | Y | V | C-Y-Y |
| IK1_23_C10 | L-V | W | F-V | N | Q-N | S-A | I | D | V | C-Y-Y |
| IK1_40_A10 | L-V | R | F-V | N | Y-M | S-A | I | Y | V | C-Y-Y |

### Example 12

### Evaluation of Oligomerization and Aggregation at Physiological pHs

This experiment evaluated the efficacy of the IFN of the present invention with respect to stability at certain pHs. More specifically, with the purpose of decreasing dimerization and higher order oligomerization of the molecule at pH ranges close to physiological ranges in order to determine aggregation levels. Thus, the relative rates of aggregation of XENP342 (C17S background, SEQ ID NO: 31) and XENP806 (L5Q/F8E/C17S variant, SEQ ID NO: 20) were measured.

The proteins were purified by Ni-capture of His-tagged Gu-solubilized inclusion bodies released from bacteria. Ni-elution fractions were refolded and cleaved with thrombin, followed by application to a semi-prep scale C4 protein reversed phase column. Elution fractions identified to have the target were repurified on an analytical scale column with the same matrix. Fractions were ‘speed-vac’-treated to remove acetonitrile prior to use.

Interferon aggregation was examined using dynamic light scattering measurements on a Malvern Zetasizer DLS (Malvern Instruments). Samples were analyzed by DLS initially, and then again after incubation under conditions designed to promote aggregation (9 hours at 37° C.). Neat samples measuring pH 1.6 due to residual TFA were diluted into either phosphate-citrate buffer resulting in pH 3, or glycine-NaOH (both 200 mM in buffer components, 300 mM NaCl) buffer resulting in pH 6±1.

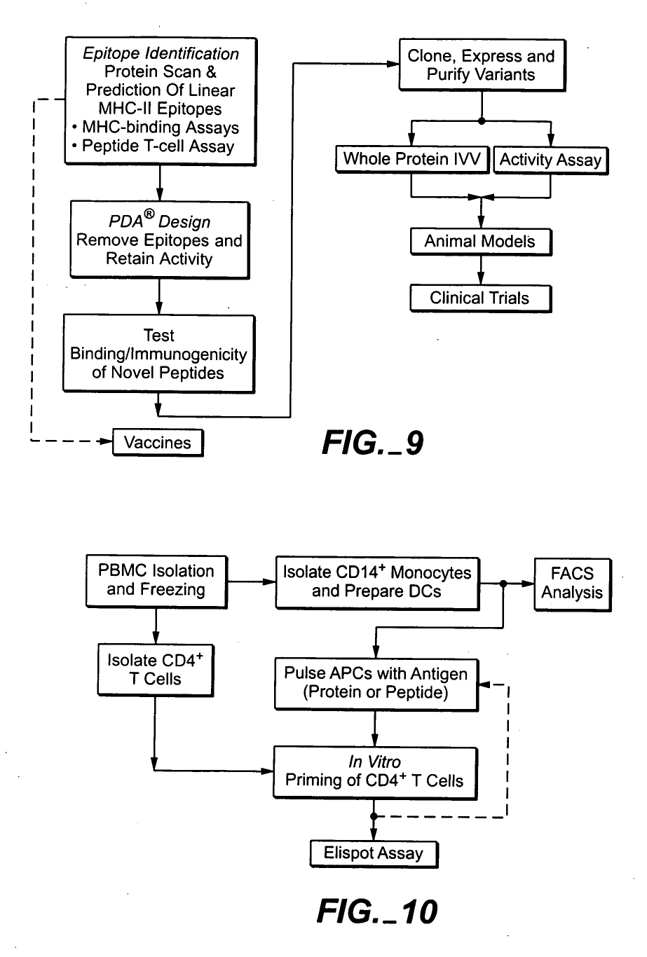

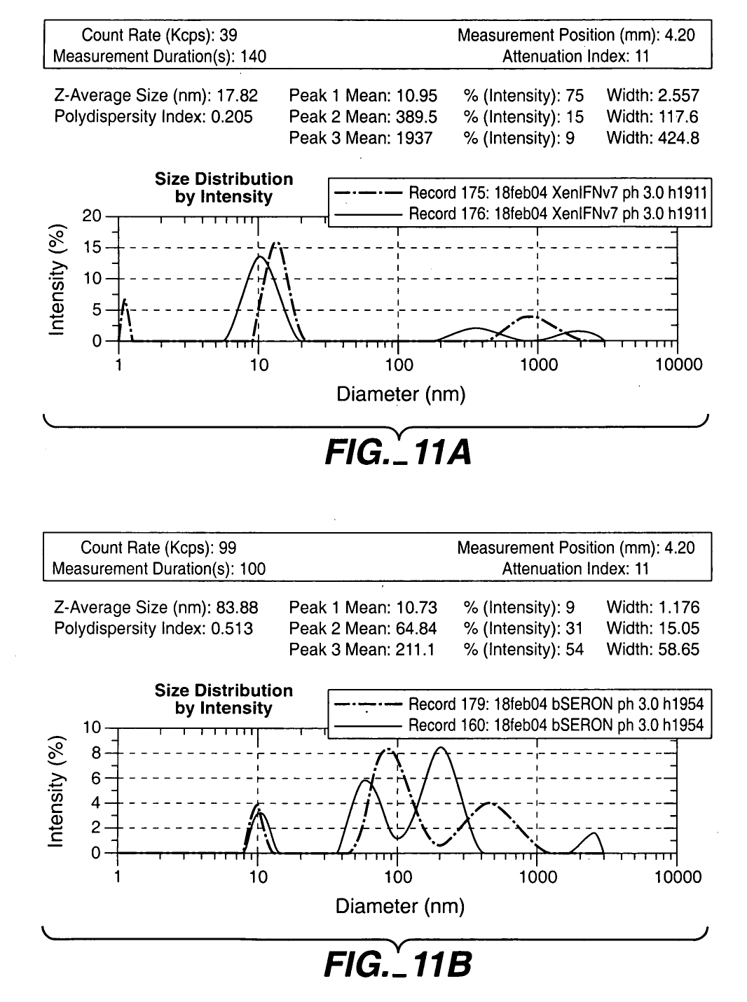

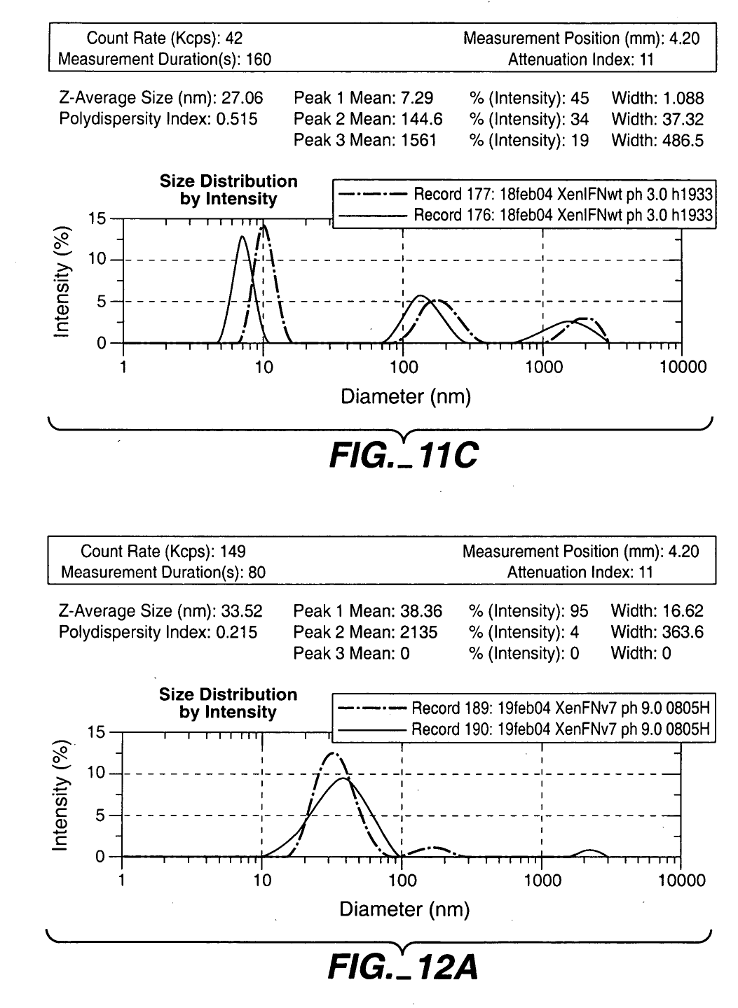

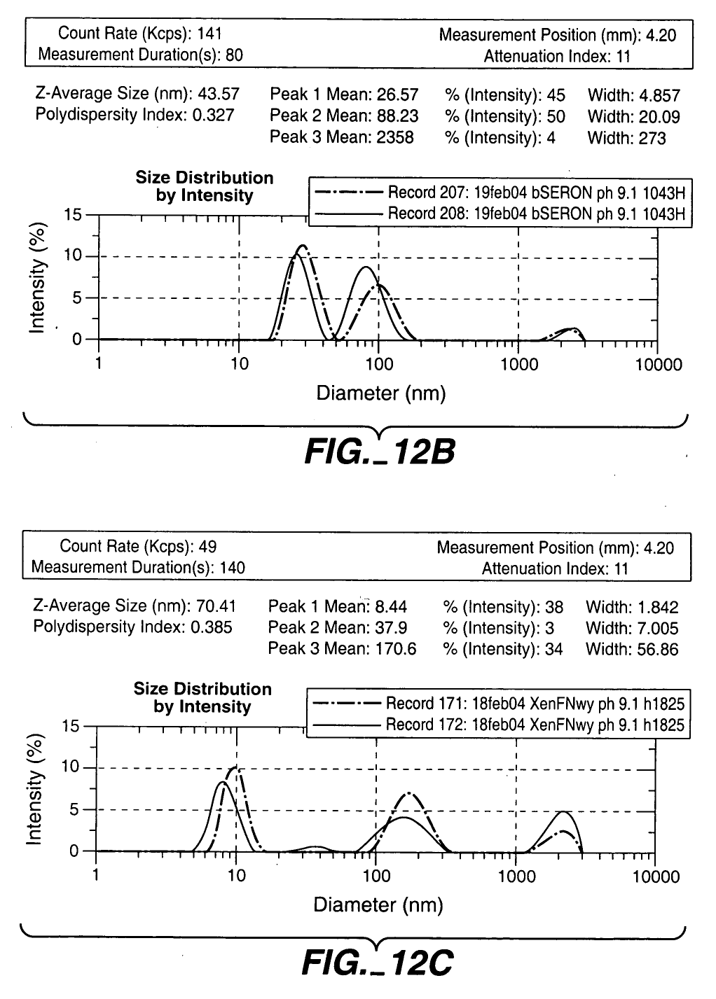

Data shown in FIGS. 9 and 10 are from two sequential measurements (green and red trace) on each sample tested, with no disturbance of the sample between measurements. In order to minimize perturbation, samples were not filtered or centrifuged.

Data presented in FIG. 9 are from pH 3 samples, and FIG. 10 shows samples at around pH 6. The data. The commercial interferon (BetaSeron®, Schering AG/Berlex) (middle graph in FIGS. 9 and 10) aggregates much more rapidly than XENP806 and qualitatively shows more aggregation than the interferon of the present invention.

***examples from provisional

### Example 13

### Identification of MHC-binding Agretopes in Interferon Beta

Matrix method calculations (Sturniolo, supra) were conducted using the parent interferon beta sequence shown in SEQ_ID_NO:1.

Agretopes were predicted for the following alleles, each of which is present in at least 1% of the US population: DRB1*0101, DRB1*0102, DRB1*0301, DRB1*0401, DRB1*0402, DRB1*0404, DRB1*0405, DRB1*0408, DRB1*0701, DRB1*0801, DRB1*1101, DRB1*1102, DRB1*1301, DRB1*1302, DRB1*1501, and DRB1*1502.

For each 9-mer that is predicted to bind to at least one allele at a 5% threshold, the number of alleles that are hit at 1%, 3% and 5% thresholds were given, as well as the percent of the US population that are predicted to react to the 9-mer. The worst 9-mers are shown in bold. They are predicted to be immunogenic in at least 10% of the US population, using a 1% threshold.

TABLE 26

Predicted MHC-binding agretopes in interferon beta.
The number of alleles and
percent of population hit at 1%, 3%, and 5%
thresholds are shown. Especially preferred
agretopes are predicted to affect at least 10% of
the population, using a 1% threshold.

Agretope

| | | | | | | | | |
|---|---|---|---|---|---|---|---|---|
| number | Residues | Sequence | 1% hits | 3% hits | 5% hits | 1% pop | 3% pop | 5% pop |

| | | | | | | | | |
|---|---|---|---|---|---|---|---|---|
| 1 | 3-11 | YNLLGFLQR | 0 | 0 | 1 | 0.0% | 0.0% | 11.4% |
| 2 | 5-13 | LLGFLQRSS | 0 | 3 | 4 | 0.0% | 19.9% | 21.2% |
| 3 | 8-16 | FLQRSSNFQ | 0 | 2 | 2 | 0.0% | 6.7% | 6.7% |
| 4 | 9-17 | LQRSSNFQC | 0 | 0 | 2 | 0.0% | 0.0% | 7.5% |
| 5 | 15-23 | FQCQKLLWQ | 0 | 1 | 1 | 0.0% | 11.4% | 11.4% |
| 6 | 22-30 | WQLNGRLEY | 2 | 3 | 5 | 19.3% | 20.9% | 28.3% |
| 7 | 30-38 | YCLKDRMNF | 0 | 2 | 2 | 0.0% | 13.5% | 13.5% |
| 8 | 36-44 | MNFDIPEEI | 1 | 1 | 1 | 21.3% | 21.3% | 21.3% |
| 9 | 47-55 | LQQFQKEDA | 0 | 0 | 1 | 0.0% | 0.0% | 1.7% |
| 10 | 57-65 | LTIYEMLQN | 0 | 2 | 2 | 0.0% | 24.1% | 24.1% |
| 11 | 60-68 | YEMLQNIFA | 2 | 7 | 7 | 15.0% | 40.2% | 40.2% |
| 12 | 63-71 | LQNIFAIFR | 0 | 1 | 1 | 0.0% | 5.0% | 5.0% |
| 13 | 70-78 | FRQDSSSTG | 0 | 1 | 3 | 0.0% | 14.0% | 33.5% |
| 14 | 79-87 | WNETIVENL | 0 | 0 | 1 | 0.0% | 0.0% | 24.7% |
| 15 | 95-103 | INHLKTVLE | 1 | 1 | 1 | 1.8% | 1.8% | 1.8% |
| 16 | 122-130 | LKRYYGRIL | 0 | 2 | 2 | 0.0% | 24.1% | 24.1% |
| 17 | 125-133 | YYGRILHYL | 0 | 0 | 1 | 0.0% | 0.0% | 5.1% |
| 18 | 129-137 | ILHYLKAKE | 1 | 1 | 1 | 5.1% | 5.1% | 5.1% |
| 19 | 130-138 | LHYLKAKEY | 0 | 0 | 1 | 0.0% | 0.0% | 5.0% |
| 20 | 143-151 | WTIVRVEIL | 1 | 1 | 1 | 24.7% | 24.7% | 24.7% |
| 21 | 145-153 | IVRVEILRN | 1 | 3 | 5 | 4.5% | 19.7% | 39.0% |
| 22 | 146-154 | VRVEILRNF | 0 | 1 | 2 | 0.0% | 10.5% | 18.5% |
| 23 | 148-156 | VEILRNFYF | 0 | 1 | 4 | 0.0% | 5.9% | 29.3% |
| 24 | 151-159 | LRNFYFINR | 1 | 2 | 2 | 22.6% | 24.1% | 24.1% |
| 25 | 154-162 | FYFINRLTG | 1 | 3 | 5 | 11.4% | 17.1% | 28.3% |
| 26 | 156-164 | FINRLTGYL | 1 | 1 | 1 | 5.1% | 5.1% | 5.1% |
| 27 | 157-165 | INRLTGYLR | 0 | 0 | 1 | 0.0% | 0.0% | 5.0% |

Alleles that are predicted as “hits” for each of the agretopes above are shown in the table below. “1” indicates a hit using a 1% threshold, “3” indicates a hit using a 3% threshold, and “5” indicates a hit using a 5% threshold.

Table 27. Predicted MHC-binding agretopes in interferon beta. DRB1 alleles that are predicted to bind to each allele at 1%, 3%, and 5% cutoffs are marked with “1”, “3”, or “5”, respectively.

TABLE 27
DRB1 alleles predicted to bind MHC agretopes in interferon beta.

| Agretope | | | | | | | | | | | | | | | | | |
|---|---|---|---|---|---|---|---|---|---|---|---|---|---|---|---|---|---|
| number | 0101 | 0102 | 0301 | 0401 | 0402 | 0404 | 0405 | 0408 | 0701 | 0801 | 1101 | 1102 | 1104 | 1301 | 1302 | 1501 | 1502 |
| 6 | — | — | — | — | — | — | — | — | — | 5 | 1 | — | 5 | — | 1 | — | 3 |
| 8 | — | — | 1 | — | — | — | — | — | — | — | — | — | — | — | — | — | — |
| 11 | 3 | — | — | 1 | — | 3 | 3 | 1 | — | — | 3 | — | — | — | — | — | 3 |
| 20 | — | — | — | — | — | — | — | — | 1 | — | — | — | — | — | — | — | — |
| 24 | — | — | — | — | — | — | — | — | — | — | — | — | — | — | — | 1 | 3 |
| 25 | — | — | — | — | — | — | — | — | — | 5 | 1 | — | 3 | — | 5 | — | 3 |

### Example 14

### Identification of Suitable Less Immunogenic Sequences for MHC-binding Agretopes in Interferon beta: BLOSUM Method

MHC-binding agretopes that were predicted to bind alleles present in at least 10% of the US population, using a 1% threshold, were analyzed to identify suitable less immunogenic variants.

At each agretope, all possible combinations of amino acid substitutions were considered, with the following requirements: (1) each substitution has a score of 0 or greater in the BLOSUM62 substitution matrix, (2) each substitution is capable of conferring reduced binding to at least one of the MHC alleles considered, and (3) once sufficient substitutions are incorporated to prevent any allele hits at a 1% threshold, no additional substitutions are added to that sequence.

Alternate sequences were scored for immunogenicity and structural compatibility. Preferred alternate sequences were defined to be those sequences that are not predicted to bind to any of the 17 MHC alleles tested above using a 1% threshold, and that have a total BLOSUM62 score that is at least 85% of the wild type score.

Table 28. Suitable less immunogenic variants of agretope 6 (residues 22-30). B(wt) is the BLOSUM62 score of the wild type 9-mer, I(alt) is the percent of the US population containing one or more MHC alleles that are predicted to bind the alternate 9-mer at a 1% threshold, and B(alt) is the BLOSUM62 score of the alternate 9-mer.

TABLE 28

Suitable less immunogenic variants
of agretope 6 (residues 22-30).

| | |
|---|---|
| Variant | WT |

| | | | | | |
|---|---|---|---|---|---|
| Sequence ID | sequence | I(alt) | B(alt) | sequence | B(wt) |

| | | | | | |
|---|---|---|---|---|---|
| SEQ_ID:1 | WSLNGRLEY | 0 | 48 | WQLNGRLEY | 53 |
| SEQ_ID:2 | WNLNGRLEY | 0 | 48 | WQLNGRLEY | 53 |
| SEQ_ID:3 | WDLNGRLEY | 0 | 48 | WQLNGRLEY | 53 |
| SEQ_ID:4 | WELNGRLEY | 0 | 50 | WQLNGRLEY | 53 |
| SEQ_ID:5 | WHLNGRLEY | 0 | 48 | WQLNGRLEY | 53 |
| SEQ_ID:6 | WKVNGRLEY | 0 | 46 | WQLNGRLEY | 53 |
| SEQ_ID:7 | WQVNGRLEY | 0 | 50 | WQLNGRLEY | 53 |
| SEQ_ID:8 | WQFSGRLEY | 0 | 44 | WQLNGRLEY | 53 |
| SEQ_ID:9 | WQFTGRLEY | 0 | 43 | WQLNGRLEY | 53 |
| SEQ_ID:10 | WQFGGRLEY | 0 | 43 | WQLNGRLEY | 53 |
| SEQ_ID:11 | WQLSGRLEY | 0 | 48 | WQLNGRLEY | 53 |
| SEQ_ID:12 | WQLTGRLEY | 0 | 47 | WQLNGRLEY | 53 |
| SEQ_ID:13 | WQLGGRLEY | 0 | 47 | WQLNGRLEY | 53 |
| SEQ_ID:14 | WQLNSQLEY | 0 | 43 | WQLNGRLEY | 53 |

Table 29. Suitable less immunogenic variants of agretope 8 (residues 36-44). B(wt) is the BLOSUM62 score of the wild type 9-mer, I(alt) is the percent of the US population containing one or more MHC alleles that are predicted to bind the alternate 9-mer at a 1% threshold, and B(alt) is the BLOSUM62 score of the alternate 9-mer.

TABLE 29

Suitable less immunogenic variants
of agretope 8 (residues 36-44).

| | |
|---|---|
| Variant | WT |

| | | | | | |
|---|---|---|---|---|---|
| Sequence ID | sequence | I(alt) | B(alt) | sequence | B(wt) |

| | | | | | |
|---|---|---|---|---|---|
| SEQ_ID:15 | QSFDIPEEI | 0 | 39 | MNFDIPEEI | 48 |
| SEQ_ID:16 | QDFDIPEEI | 0 | 39 | MNFDIPEEI | 48 |
| SEQ_ID:17 | MSFDIPEEI | 0 | 43 | MNFDIPEEI | 48 |
| SEQ_ID:18 | MTFDIPEEI | 0 | 42 | MNFDIPEEI | 48 |
| SEQ_ID:19 | MGFDIPEEI | 0 | 42 | MNFDIPEEI | 48 |
| SEQ_ID:20 | MDFDIPEEI | 0 | 43 | MNFDIPEEI | 48 |
| SEQ_ID:21 | MEFDIPEEI | 0 | 42 | MNFDIPEEI | 48 |
| SEQ_ID:22 | MNYSIPEEI | 0 | 39 | MNFDIPEEI | 48 |
| SEQ_ID:23 | MNYNIPEEI | 0 | 40 | MNFDIPEEI | 48 |
| SEQ_ID:24 | MNYEIPEEI | 0 | 41 | MNFDIPEEI | 48 |
| SEQ_ID:25 | MNYQIPEEI | 0 | 39 | MNFDIPEEI | 48 |
| SEQ_ID:26 | MNFSIPEEI | 0 | 42 | MNFDIPEEI | 48 |
| SEQ_ID:27 | MNFNIPEEI | 0 | 43 | MNFDIPEEI | 48 |
| SEQ_ID:28 | MNFEIPEEI | 0 | 44 | MNFDIPEEI | 48 |
| SEQ_ID:29 | MNFQIPEEI | 0 | 42 | MNFDIPEEI | 48 |
| SEQ_ID:30 | MNFDIPESL | 0 | 41 | MNFDIPEEI | 48 |
| SEQ_ID:31 | MNFDIPESV | 0 | 42 | MNFDIPEEI | 48 |
| SEQ_ID:32 | MNFDIPENL | 0 | 41 | MNFDIPEEI | 48 |
| SEQ_ID:33 | MNFDIPENV | 0 | 42 | MNFDIPEEI | 48 |
| SEQ_ID:34 | MNFDIPEDL | 0 | 43 | MNFDIPEEI | 48 |
| SEQ_ID:35 | MNFDIPEDV | 0 | 44 | MNFDIPEEI | 48 |
| SEQ_ID:36 | MNFDIPEQL | 0 | 43 | MNFDIPEEI | 48 |
| SEQ_ID:37 | MNFDIPEQV | 0 | 44 | MNFDIPEEI | 48 |
| SEQ_ID:38 | MNFDIPEHL | 0 | 41 | MNFDIPEEI | 48 |
| SEQ_ID:39 | MNFDIPEHV | 0 | 42 | MNFDIPEEI | 48 |
| SEQ_ID:40 | MNFDIPERL | 0 | 41 | MNFDIPEEI | 48 |
| SEQ_ID:41 | MNFDIPERV | 0 | 42 | MNFDIPEEI | 48 |
| SEQ_ID:42 | MNFDIPEKL | 0 | 42 | MNFDIPEEI | 48 |
| SEQ_ID:43 | MNFDIPEKV | 0 | 43 | MNFDIPEEI | 48 |
| SEQ_ID:44 | MNFDIPEEL | 0 | 46 | MNFDIPEEI | 48 |
| SEQ_ID:45 | MNFDIPEEV | 0 | 47 | MNFDIPEEI | 48 |

Table 30 Suitable less immunogenic variants of agretope 11 (residues 60-68). B(wt) is the BLOSUM62 score of the wild type 9-mer, I(alt) is the percent of the US population containing one or more MHC alleles that are predicted to bind the alternate 9-mer at a 1% threshold, and B(alt) is the BLOSUM62 score of the alternate 9-mer.

TABLE 30

Suitable less immunogenic variants
of agretope 11 (residues 60-68).

| | |
|---|---|
| Variant | WT |

| | | | | | |
|---|---|---|---|---|---|
| Sequence ID | sequence | I(alt) | B(alt) | sequence | B(wt) |

| | | | | | |
|---|---|---|---|---|---|
| SEQ_ID:46 | HDMLQNIFA | 0 | 38 | YEMLQNIFA | 46 |
| SEQ_ID:47 | YSQLQNIFA | 0 | 37 | YEMLQNIFA | 46 |
| SEQ_ID:48 | YSLLQNIFA | 0 | 38 | YEMLQNIFA | 46 |
| SEQ_ID:49 | YSVLQNIFA | 0 | 37 | YEMLQNIFA | 46 |
| SEQ_ID:50 | YSFLQNIFA | 0 | 37 | YEMLQNIFA | 46 |
| SEQ_ID:51 | YEQLQNIFA | 0 | 42 | YEMLQNIFA | 46 |
| SEQ_ID:52 | YEMLQNIYT | 0 | 39 | YEMLQNIFA | 46 |
| SEQ_ID:53 | YEMLQNIWT | 0 | 37 | YEMLQNIFA | 46 |
| SEQ_ID:54 | YEMLQNIFT | 0 | 42 | YEMLQNIFA | 46 |

Table 31. Suitable less immunogenic variants of agretope 20 (residues 143-151). B(wt) is the BLOSUM62 score of the wild type 9-mer, I(alt) is the percent of the US population containing one or more MHC alleles that are predicted to bind the alternate 9-mer at a 1% threshold, and B(alt) is the BLOSUM62 score of the alternate 9-mer.

TABLE 31

Suitable less immunogenic variants
of agretope 20 (residues 143-151).

| | |
|---|---|
| Variant | WT |

| | | | | | |
|---|---|---|---|---|---|
| Sequence ID | sequence | I(alt) | B(alt) | sequence | B(wt) |

| | | | | | |
|---|---|---|---|---|---|
| SEQ_ID:55 | WSIVRVEIL | 0 | 42 | WTIVRVEIL | 46 |
| SEQ_ID:56 | WTIVRVSIL | 0 | 41 | WTIVRVEIL | 46 |
| SEQ_ID.57 | WTIVRVEMM | 0 | 41 | WTIVRVEIL | 46 |
| SEQ_ID:58 | WTIVRVEMV | 0 | 40 | WTIVRVEIL | 46 |
| SEQ_ID:59 | WTIVRVEMF | 0 | 39 | WTIVRVEIL | 46 |
| SEQ_ID:60 | WTIVRVELF | 0 | 40 | WTIVRVEIL | 46 |
| SEQ_ID:61 | WTIVRVEVF | 0 | 41 | WTIVRVEIL | 46 |
| SEQ_ID:62 | WTIVRVEFF | 0 | 38 | WTIVRVEIL | 46 |
| SEQ_ID:63 | WTIVRVEIM | 0 | 44 | WTIVRVEIL | 46 |
| SEQ_ID:64 | WTIVRVEIV | 0 | 43 | WTIVRVEIL | 46 |
| SEQ_ID:65 | WTIVRVEIF | 0 | 42 | WTIVRVEIL | 46 |

Table 32. Suitable less immunogenic variants of agretope 24 (residues 151-159). B(wt) is the BLOSUM62 score of the wild type 9-mer, I(alt) is the percent of the US population containing one or more MHC alleles that are predicted to bind the alternate 9-mer at a 1% threshold, and B(alt) is the BLOSUM62 score of the alternate 9-mer.

TABLE 33

Suitable less immunogenic variants
of agretope 24 (residues 151-159).

| | |
|---|---|
| Variant | WT |

| | | | | | |
|---|---|---|---|---|---|
| Sequence ID | sequence | I(alt) | B(alt) | sequence | B(wt) |

| | | | | | |
|---|---|---|---|---|---|
| SEQ_ID:66 | MNNFYFINR | 0 | 42 | LRNFYFINR | 49 |
| SEQ_ID:67 | MENFYFINR | 0 | 42 | LRNFYFINR | 49 |
| SEQ_ID:68 | MQNFYFINR | 0 | 43 | LRNFYFINR | 49 |
| SEQ_ID:69 | MHNFYFINR | 0 | 42 | LRNFYFINR | 49 |
| SEQ_ID:70 | MKNFYFINR | 0 | 44 | LRNFYFINR | 49 |
| SEQ_ID:71 | LNNFYFINR | 0 | 44 | LRNFYFINR | 49 |
| SEQ_ID:72 | LENFYFINR | 0 | 44 | LRNFYFINR | 49 |
| SEQ_ID:73 | LQNFYFINR | 0 | 45 | LRNFYFINR | 49 |
| SEQ_ID:74 | LHNFYFINR | 0 | 44 | LRNFYFINR | 49 |
| SEQ_ID:75 | LKNFYFINR | 0 | 46 | LRNFYFINR | 49 |
| SEQ_ID:76 | LRSFYFINR | 0 | 44 | LRNFYFINR | 49 |
| SEQ_ID:77 | LRTFYFINR | 0 | 43 | LRNFYFINR | 49 |
| SEQ_ID:78 | LRGFYFINR | 0 | 43 | LRNFYFINR | 49 |
| SEQ_ID:79 | LRDFYFINR | 0 | 44 | LRNFYFINR | 49 |
| SEQ_ID:80 | LREFYFINR | 0 | 43 | LRNFYFINR | 49 |
| SEQ_ID:81 | LRQFYFINR | 0 | 43 | LRNFYFINR | 49 |
| SEQ_ID:82 | LRHFYFINR | 0 | 44 | LRNFYFINR | 49 |
| SEQ_ID:83 | LRKFYFINR | 0 | 43 | LRNFYFINR | 49 |
| SEQ_ID:84 | LRNMYFINR | 0 | 43 | LRNFYFINR | 49 |
| SEQ_ID:85 | LRNIYFINR | 0 | 43 | LRNFYFINR | 49 |
| SEQ_ID:86 | LRNLYFINR | 0 | 43 | LRNFYFINR | 49 |
| SEQ_ID:87 | LRNFHYVNR | 0 | 40 | LRNFYFINR | 49 |
| SEQ_ID:88 | LRNFYFISQ | 0 | 40 | LRNFYFINR | 49 |
| SEQ_ID:89 | LRNFYFISK | 0 | 41 | LRNFYFINR | 49 |
| SEQ_ID:90 | LRNFYFITK | 0 | 40 | LRNFYFINR | 49 |
| SEQ_ID:91 | LRNFYFIGK | 0 | 40 | LRNFYFINR | 49 |
| SEQ_ID:92 | LRNFYFIDK | 0 | 41 | LRNFYFINR | 49 |
| SEQ_ID:93 | LRNFYFIEK | 0 | 40 | LRNFYFINR | 49 |
| SEQ_ID:94 | LRNFYFIQK | 0 | 40 | LRNFYFINR | 49 |
| SEQ_ID:95 | LRNFYFIHK | 0 | 41 | LRNFYFINR | 49 |
| SEQ_ID:96 | LRNFYFIRK | 0 | 40 | LRNFYFINR | 49 |
| SEQ_ID:97 | LRNFYFIKK | 0 | 40 | LRNFYFINR | 49 |
| SEQ_ID:98 | LRNFYFINE | 0 | 44 | LRNFYFINR | 49 |
| SEQ_ID:99 | LRNFYFINQ | 0 | 45 | LRNFYFINR | 49 |
| SEQ_ID:100 | LRNFYFINK | 0 | 46 | LRNFYFINR | 49 |

Table 34. Suitable less immunogenic variants of agretope 25 (residues 154-162). B(wt) is the BLOSUM62 score of the wild type 9-mer, I(alt) is the percent of the US population containing one or more MHC alleles that are predicted to bind the alternate 9-mer at a 1% threshold, and B(alt) is the BLOSUM62 score of the alternate 9-mer.

TABLE 34

Suitable less immunogenic variants
of agretope 25 (residues 154-162).

| | |
|---|---|
| Variant | WT |

| Sequence ID | sequence | I(alt) | B(alt) | sequence | B(wt) |
|---|---|---|---|---|---|
| SEQ_ID:101 | FYFISQLTG | 0 | 40 | FYFINRLTG | 49 |

### Example 15

### Identification of Suitable Less Immunogenic Sequences for MHC-binding Agretopes in Interferon Beta: PDA® Technology

MHC-binding agretopes that were predicted to bind alleles present in at least 10% of the US population, using a 1% threshold, were analyzed using PDA® technology to identify suitable less immunogenic variants.

Each position in the agretopes of interest were analyzed to identify a subset of amino acid substitutions that are potentially compatible with maintaining the structure and function of the protein. This step may be performed in several ways, including PDA® calculations or visual inspection by one skilled in the art. Sequences may be generated that contain all possible combinations of amino acids that were selected for consideration at each position. Matrix method calculations were then used to determine the immunogenicity of each sequence. The results were analyzed to identify sequences that are predicted to have significantly decreased immunogenicity. PDA® calculations may be performed to determine which of the minimally immunogenic sequences are compatible with maintaining the structure and function of the protein.

Alternate sequences were scored for immunogenicity and structural compatibility. Preferred alternate sequences were defined to be those sequences in which (1) the central 9-mer agretope is not predicted to bind to any of the 17 MHC alleles tested above using a 5% threshold, (2) overlapping 9-mer agretopes are not predicted to bind any of the 17 MHC alleles tested more tightly than the analogous 9-mer in wild type interferon beta, and (3) the total energy, determined using PDA® technology calculations, that is at most 1 kcal mol⁻¹ worse than the energy of the wild type interferon beta.

### Example 16

### Analysis of Immunogenic Sequences in Interferon Beta Solubility Variants

A set of interferon beta variants were engineered for enhanced solubility. These variants comprise hydrophobic to polar substitutions at solvent exposed positions that are not functionally required.

Table 35. Specific activity data for interferon-beta variants. The sequence for residues 5, 8, 47, 111, 116, and 120 is given for each variant, along with the total number of mutations, the EC50, and the ratio of the wild type to variant EC50. Variant IFN1_{—}1 is the interferon beta wild type with the C17S substitution.

TABLE 35

Sequence and activity of
interferon beta solubility variants.

| | |
|---|---|
| EC50 | EC50 wt/ |

| | | | | | | | | | |
|---|---|---|---|---|---|---|---|---|---|
| Variant | 5 | 8 | 47 | 111 | 116 | 120 | # mut | (log ng/ml) | EC50 var |

| | | | | | | | | | |
|---|---|---|---|---|---|---|---|---|---|
| IFN1_1 | L | F | L | F | L | L | 0 | 5.306 | 1.0 |
| IFB1_2 | Q | F | L | F | L | L | 1 | 0.428 | 12.4 |
| IFB1_7 | Q | E | L | F | L | L | 2 | 0.179 | 29.6 |
| IFB1_15 | Q | E | L | N | L | L | 3 | 0.319 | 16.6 |
| IFB1_23 | Q | E | L | F | E | L | 3 | 0.277 | 19.2 |
| IFB1_36 | L | E | L | N | E | L | 3 | 0.294 | 18.0 |
| IFB1_39 | Q | E | L | N | E | L | 4 | 0.193 | 27.5 |
| IFB1_64 | Q | E | K | N | E | R | 6 | 0.240 | 22.1 |

Table 36. Comparison of MHC agretopes in interferon beta solubility variants. Potential agretopes that include residues that were altered in one or more of the solubility variants are shown, along with the fraction of the population for which each agretope is a hit using a 3% threshold.

TABLE 36

Comparison of MHC agretopes in
interferon beta solubility variants

| | | | | | | | | | | |
|---|---|---|---|---|---|---|---|---|---|---|
| mutations | residues | sequence | wt | v2 | v7 | v15 | v23 | v36 | v39 | v64 |

| | | | | | | | | | | |
|---|---|---|---|---|---|---|---|---|---|---|
| L5Q,F8E | 1-9 | MSYNLLGFL | 0.00 | 0.00 | 0.00 | 0.00 | 0.00 | 0.00 | 0.00 | 0.00 |
| L5Q,F8E | 3-11 | YNLLGFLQR | 0.00 | 0.00 | 0.00 | 0.00 | 0.00 | 0.00 | 0.00 | 0.00 |
| L5Q,F8E | 5-13 | LLGFLQRSS | 0.20 | 0.00 | 0.00 | 0.00 | 0.00 | 0.00 | 0.00 | 0.00 |
| F8E | 6-14 | LGFLQRSSN | 0.00 | 0.00 | 0.00 | 0.00 | 0.00 | 0.00 | 0.00 | 0.00 |
| F8E | 8-16 | FLQRSSNFQ | 0.07 | 0.07 | 0.00 | 0.00 | 0.00 | 0.00 | 0.00 | 0.00 |
| L47K | 40-48 | IPEEIKQLQ | 0.00 | 0.00 | 0.00 | 0.00 | 0.00 | 0.00 | 0.00 | 0.00 |
| L47K | 44-52 | IKQLQQFQK | 0.00 | 0.00 | 0.00 | 0.00 | 0.00 | 0.00 | 0.00 | 0.00 |
| L47K | 47-55 | LQQFQKEDA | 0.00 | 0.00 | 0.00 | 0.00 | 0.00 | 0.00 | 0.00 | 0.00 |
| F111N | 106-114 | LEKEDFTRG | 0.00 | 0.00 | 0.00 | 0.00 | 0.00 | 0.00 | 0.00 | 0.00 |
| F111N,L116E | 111-119 | FTRGKLMSS | 0.00 | 0.00 | 0.00 | 0.00 | 0.00 | 0.00 | 0.00 | 0.00 |
| L116E,L120R | 116-124 | LMSSLHLKR | 0.00 | 0.00 | 0.00 | 0.00 | 0.00 | 0.00 | 0.00 | 0.00 |
| L120R | 117-125 | MSSLHLKRY | 0.00 | 0.00 | 0.00 | 0.00 | 0.00 | 0.00 | 0.00 | 0.00 |
| L120R | 120-128 | LHLKRYYGR | 0.00 | 0.00 | 0.00 | 0.00 | 0.00 | 0.00 | 0.00 | 0.00 |

As can be seen, many of the solubility variants are predicted to have reduced immunogenicity relative to wild type interferon beta.

1. A variant type 1 interferon beta (IFN-β) protein exhibiting modified immunogenicity as compared to a wild type IFN-β, said variant comprising at least one modification at a position selected from the group consisting of 1,2, 3, 4, 5, 6, 8, 9, 12, 15, 16, 22, 28, 30, 32, 36, 42, 43, 46, 47, 48, 49, 51, 92, 93, 96,100, 101, 104, 111, 113, 116, 117,120, 121, 124,130, 148, and 155, wherein said modifications to residues 5, 8, 15, 47, 111, 116, and 120 are substitution mutations selected from the group consisting of alanine, arginine, aspartic acid, asparagine, glutamic acid, glutamine, glycine, histidine, and lysine, and said modifications to residues 22, 28, 30, 32, 36, 92, 130, 148, and 155 are selected from the group including alanine, arginine, aspartic acid, asparagine, glutamic acid, glutamine, glycine, histidine, serine, threonine and lysine.

2. A variant IFN-β according to claim 1 wherein said modified immunogenicity is reduced immunogenicity.

3. A variant IFN-β according to claim 2 wherein said reduced immunogenicity is increased solubility..

4. A variant IFN-β according to claim 1 wherein said protein demonstrates reduced binding to at least one human class II MHC allele.

5. A variant IFN-β according to claim 1 wherein said modified immunogenicity is increased immunogenicity.

6. A variant type 1 interferon alpha (IFN-α) protein exhibiting modified immunogenicity as compared to a wild type IFN-α comprising at least one modification at a position selected from the group consisting of16, 27, 30, 89, 100, 110, 111, 117, 128, and 161, wherein said modifications are substitution mutations selected from the group consisting of alanine, arginine, aspartic acid, asparagine, glutamic acid, glutamine, glycine, histidine, serine, threonine, and lysine.

7. A variant IFN-α according to claim 6 wherein said modified immunogenicity is reduced immunogenicity.

8. A variant IFN-α according to claim 7 wherein said reduced immunogenicity is increased solubility.

9. A variant IFN-α according to claim 7 wherein said protein demonstrates reduced binding to at least one human class II MHC allele.

10. A variant IFN-α according to claim 6 wherein said modified immunogenicity is increased immunogenicity.

11. A variant type 1 interferon kappa (IFN-κ) protein exhibiting modified immunogenicity as compared to a wild type IFN-κ comprising at least one modification at a position selected from the group consisting of 1, 5, 8, 15, 18, 28, 30, 33, 37, 46, 48, 52, 65, 68, 76, 79, 89, 97, 112, 115, 120,127, 133, 151, 161, 168, and 171, wherein said modifications are substitution mutations selected from the group consisting of alanine, arginine, aspartic acid, asparagine, glutamic acid, glutamine, glycine, histidine, serine, threonine, and lysine.

12. A variant IFN-κ according to claim 11 wherein said modified immunogenicity is reduced immunogenicity.

13. A variant IFN-κ according to claim 12 wherein said reduced immunogenicity is increased solubility.

14. A variant IFN-κ according to claim 12 wherein said protein demonstrates reduced binding to at least one human class II MHC allele.

15. A variant IFN-κ according to claim 11 wherein said modified immunogenicity is increased immunogenicity.
